SEQUENCE LISTING (150 sequences)

SEQ ID NO: 1 - DNA, 483 bp, Artificial Sequence
  CLIBASIA_00420 Full-length nucleotide sequence
atgcaagctcggtgctttttatccttatcgttcctttttcccctcgttgctgccatagctgtggtttcttgtagtgccttgtcagtcgttgttgatagcagtgagttattgccagaaccaatggatataacaggatattgggaagatgataatggaattttatcgtattttcaaaaagatggaaaattcaaaaccatctctacagatggatccagtagcgttttagcaacaggctcttatcatgttaaaattaatcaagacgtagaaattaagctcacctctttaattcgcaatacttcaggaaagattcaatgtcagttcttagattcaaataaactcaactgtcttgcaaaagatcaaaaacagttctatttgcgacgcacacatctaacagaattgccttcctctaaaccacaggacgatgtcgctgagattcctgaggatcctaccaccccttctacgtcaatcatattttataaatag

SEQ ID NO: 2 - protein, 160 aa, Artificial Sequence
  CLIBASIA_00420 full length amino acid sequence
MQARCFLSLSFLFPLVAAIAVVSCSALSVVVDSSELLPEPMDITGYWEDDNGILSYFQKDGKFKTISTDGSSSVLATGSYHVKINQDVEIKLTSLIRNTSGKIQCQFLDSNKLNCLAKDQKQFYLRRTHLTELPSSKPQDDVAEIPEDPTTPSTSIIFYK

SEQ ID NO: 3 - protein, 132 aa, Artificial Sequence
  CLIBASIA_00420 mature protein sequence
VVVDSSELLPEPMDITGYWEDDNGILSYFQKDGKFKTISTDGSSSVLATGSYHVKINQDVEIKLTSLIRNTSGKIQCQFLDSNKLNCLAKDQKQFYLRRTHLTELPSSKPQDDVAEIPEDPTTPSTSIIFYK

SEQ ID NO: 4 - DNA, 297 bp, Artificial Sequence
  CLIBASIA_00460 full length nucleotide sequence
atgcgtcatttgattttaataatgcttttatccatactaaccactaatatagctagagctcaagtttatcatatccattcgcctcgtattgcaacaaaatcttcaatacatattaagtgtcatagttgcactctaaataaacatcatatcaataaaaccccttcatcttcttccgccgtgtacaccaaaaaagaagaactaattgatgggaaaaaagcaatgatcacaacagataattttatgggaggtgaacctataacttttatcaaatatttatttgaagaagataaaaaatag

SEQ ID NO: 5 - protein, 98 aa, Artificial Sequence
  CLIBASIA_00460 full length amino acid sequence
MRHLILIMLLSILTTNIARAQVYHIHSPRIATKSSIHIKCHSCTLNKHHINKTPSSSSAVYTKKEELIDGKKAMITTDNFMGGEPITFIKYLFEEDKK

SEQ ID NO: 6 - protein, 78 aa, Artificial Sequence
  CLIBASIA_00460 mature protein sequence
QVYHIHSPRIATKSSIHIKCHSCTLNKHHINKTPSSSSAVYTKKEELIDGKKAMITTDNFMGGEPITFIKYLFEEDKK

SEQ ID NO: 7 - DNA, 156 bp, Artificial Sequence
  CLIBASIA_00470 full length nucleotide sequence
atgtataccaaaagtttgttaatggtagcttatttgttatcttcggttgcaatatcgggtgggctatgctttaaccgcccaaaaggcccgtctaaagaagaacaagcacgaatagaacaaatacgagcagaagcaagagaaaggcgctaccaataa

SEQ ID NO: 8 - protein, 51 aa, Artificial Sequence
  CLIBASIA_00470 full length amino acid sequence
MYTKSLLMVAYLLSSVAISGGLCFNRPKGPSKEEQARIEQIRAEARERRYQ

SEQ ID NO: 9 - protein, 32 aa, Artificial Sequence
  CLIBASIA_00470 mature protein sequence
GGLCFNRPKGPSKEEQARIEQIRAEARERRYQ

SEQ ID NO: 10 - DNA, 294 bp, Artificial Sequence
  CLIBASIA_00525 full length nucleotide sequence
atgaaaaaaacacaattacttttgcctcttttaacactcttaagcagttgttctgattatgtatatgaagatgcgatcaggtctcaatttgaaaatgagatcaggtattataaatcaatgcatccgtctacgcaagacgacatagaatataatctctcagagataaaatctttcgaaaatcaaatcctggcaatatccaataaactggaaaaaggacagaagccgaaatatctgcacttaaaagaagcgatacagaaaattgtaaaaaccattgagcaaaatgagaaagagtaa

SEQ ID NO: 11 - protein, 97 aa, Artificial Sequence
  CLIBASIA_00525 full length amino acid sequence
MKKTQLLLPLLTLLSSCSDYVYEDAIRSQFENEIRYYKSMHPSTQDDIEYNLSEIKSFENQILAISNKLEKGQKPKYLHLKEAIQKIVKTIEQNEKE

SEQ ID NO: 12 - protein, 76 aa, Artificial Sequence
  CLIBASIA_00525 mature amino acid sequence
YEDAIRSQFENEIRYYKSMHPSTQDDIEYNLSEIKSFENQILAISNKLEKGQKPKYLHLKEAIQKIVKTIEQNEKE

SEQ ID NO: 13 - DNA, 294 bp, Artificial Sequence
  CLIBASIA_00530 full length nucleotide
atgcgctttaaaacaaaacaattaattttagctgttttagtaaccttattaggtagctgtgccgatgacaggattacggaattaaatacccttctcgctgaatataaagaagagaatcttaaaataagagaattacaaaaggaattgtacccggctattagcactctcgcgcacaggatggataaattacatttcgaatcagatgctttagaccctattttgagacgtatggatggtccagtaaaacacgtggaaagacggattaatagtaaaaccacaacccttgaacaataa

SEQ ID NO: 14 - protein, 97 aa, Artificial Sequence
  CLIBASIA_00530 full length amino acid
MRFKTKQLILAVLVTLLGSCADDRITELNTLLAEYKEENLKIRELQKELYPAISTLAHRMDKLHFESDALDPILRRMDGPVKHVERRINSKTTTLEQ

SEQ ID NO: 15 - protein, 76 aa, Artificial Sequence
  CLIBASIA_00530 mature protein sequence
DDRITELNTLLAEYKEENLKIRELQKELYPAISTLAHRMDKLHFESDALDPILRRMDGPVKHVERRINSKTTTLEQ

SEQ ID NO: 16 - DNA, 426 bp, Artificial Sequence
  CLIBASIA_01640 full length nucleotide sequence
atgcttcgtagttttttgatatgtttttgctttggagctatgattttttgttcaaatgtgtcttttgctggttttcgggtttgtaatggcactaaaaatttaatcggtgttgctgtagggtatcctgctgtaaaaggtggatggatgacggaaggatggtggcagattcctgggaatacttgtgaaactgttgtaaaaggtgctttgcattcacggtactattatttgtatgccgagggagtttcacatagtgagcactgggctggaaatgtgcaaatgtgtgtagggcaagatgaatttaatatcgtggatataaagaattgttatacgcgtggttatttgagagttggttttactgaatacgatacagggcaacatgagaattggacagtacaactcactgagcctgcacaagataggagataa

SEQ ID NO: 17 - protein, 141 aa, Artificial Sequence
  CLIBASIA_01640 full length amino acid
MLRSFLICFCFGAMIFCSNVSFAGFRVCNGTKNLIGVAVGYPAVKGGWMTEGWWQIPGNTCETVVKGALHSRYYYLYAEGVSHSEHWAGNVQMCVGQDEFNIVDIKNCYTRGYLRVGFTEYDTGQHENWTVQLTEPAQDRR

SEQ ID NO: 18 - protein, 118 aa, Artificial Sequence
  CLIBASIA_01640 mature protein sequence
GFRVCNGTKNLIGVAVGYPAVKGGWMTEGWWQIPGNTCETVVKGALHSRYYYLYAEGVSHSEHWAGNVQMCVGQDEFNIVDIKNCYTRGYLRVGFTEYDTGQHENWTVQLTEPAQDRR

SEQ ID NO: 19 - DNA, 246 bp, Artificial Sequence
  CLIBASIA_02215 full length nucleotide sequence
atggaagtgttagaagcccataagctggctaaagagtatgtggaacaagctaatcaacgcgttaaagaagctgaagaacaatctaatgcacggctacttaagggacttgggatggatgatcttgtccgatattttatgaatttggatagccaaaaccaagcgtttttcatcgatacaatacaaaataagtatcaagatgaaatggaagaattaagtgaatttggggagaaagtatcggattactaa

SEQ ID NO: 20 - protein, 81 aa, Artificial Sequence
  CLIBASIA_02215 full length amino acid sequence
MEVLEAHKLAKEYVEQANQRVKEAEEQSNARLLKGLGMDDLVRYFMNLDSQNQAFFIDTIQNKYQDEMEELSEFGEKVSDY

SEQ ID NO: 21 - protein, 61 aa, Artificial Sequence
  CLIBASIA_02215 mature protein sequence
VKEAEEQSNARLLKGLGMDDLVRYFMNLDSQNQAFFIDTIQNKYQDEMEELSEFGEKVSDY

SEQ ID NO: 22 - DNA, 342 bp, Artificial Sequence
  CLIBASIA_02470 full length nucleotide sequence
atggctcttaattgcaacgaaactttaatgcaagccgatatgaatcaatgcacaggaaattcttttgcactagtaaaagagaaactagaagcaacatataaaaaagtcttagaaaaagttgaaaagcatcaaagagaattatttgaaaaatcacaaatggcatgggaaatataccgaggttctgaatgcgcctttgctgcttctggagcagaagaaggaactgcacaatcaatgatttatgcgaattgtctacaaggacatgccatcgaacgaaatgagaaactagaatcctaccttacatgtccagaaggcgatctgctctgcccatttataaataattga

SEQ ID NO: 23 - protein, 113 aa, Artificial Sequence
  CLIBASIA_02470 full length amino acid sequence
MALNCNETLMQADMNQCTGNSFALVKEKLEATYKKVLEKVEKHQRELFEKSQMAWEIYRGSECAFAASGAEEGTAQSMIYANCLQGHAIERNEKLESYLTCPEGDLLCPFINN

SEQ ID NO: 24 - protein, 112 aa, Artificial Sequence
  CLIBASIA_02470 mature protein sequence
ALNCNETLMQADMNQCTGNSFALVKEKLEATYKKVLEKVEKHQRELFEKSQMAWEIYRGSECAFAASGAEEGTAQSMIYANCLQGHAIERNEKLESYLTCPEGDLLCPFINN

SEQ ID NO: 25 - DNA, 183 bp, Artificial Sequence
  CLIBASIA_03120 full length nucleotide sequence
atggcacgcactcagatcgcattggctttatctttttttatgataactcatagttattatgcgttttcgcaagatgaaattaagaagaataatcctacgttagaaaaaaagcccatcgtcctcatgaagcatgaaattcaagaaaaaaaaacccttgccgcctttacctcctttgcatcgtaa

SEQ ID NO: 26 - protein, 60 aa, Artificial Sequence
  CLIBASIA_03120 full length amino acid sequence
MARTQIALALSFFMITHSYYAFSQDEIKKNNPTLEKKPIVLMKHEIQEKKTLAAFTSFAS

SEQ ID NO: 27 - protein, 37 aa, Artificial Sequence
  CLIBASIA_03120 mature protein sequence
QDEIKKNNPTLEKKPIVLMKHEIQEKKTLAAFTSFAS

SEQ ID NO: 28 - DNA, 495 bp, Artificial Sequence
  CLIBASIA_03230 full length nucleotide sequence
atgctaatgaacttcagaatagcgatgttaatatcttttttagcgagtggctgtgttgcgcatgctcttcttacgaaaaagattgaaagtgatactgattcccgtcacgaaaaagctacgatctctctgtctgcgcatgataaggaaggatcgaaacacacaatgaatgctgagttctcggtaccaaaaaatgatgaaaagtataccatatcttctcttacgaaaaagattgaaagtgatactgatttccgtcgcgaaaaagctacgatctctctgtctgcccatgataaggaaggatcgaaacacacaatgaatgctgagttctcagtaccaaaaaatgatgaaaagtataccatatctgcatgtgcgtctgacgataaaggaaacaaaagcacgctatgtgttgagtgtccgtctccgagcactcctgggcagtatgatcttaatcattgtgccgaatgcgagaatacgacgtcaaagggtttatgtccctga

SEQ ID NO: 29 - protein, 164 aa, Artificial Sequence
  CLIBASIA_03230 full length amino acid sequence
MLMNFRIAMLISFLASGCVAHALLTKKIESDTDSRHEKATISLSAHDKEGSKHTMNAEFSVPKNDEKYTISSLTKKIESDTDFRREKATISLSAHDKEGSKHTMNAEFSVPKNDEKYTISACASDDKGNKSTLCVECPSPSTPGQYDLNHCAECENTTSKGLCP

SEQ ID NO: 30 - protein, 142 aa, Artificial Sequence
  CLIBASIA_03230 mature protein sequence
LLTKKIESDTDSRHEKATISLSAHDKEGSKHTMNAEFSVPKNDEKYTISSLTKKIESDTDFRREKATISLSAHDKEGSKHTMNAEFSVPKNDEKYTISACASDDKGNKSTLCVECPSPSTPGQYDLNHCAECENTTSKGLCP

SEQ ID NO: 31 - DNA, 204 bp, Artificial Sequence
  CLIBASIA_03695 full length nucleotide sequence
atgtctccactggctccactgggagatggttgcgagcagttagtaggtgcgacagttcatagtgagggaaagaaaagaagtcacgatgtgcccagtagttcaaatcaagaagatcaaaggggaagcccttctcctaaaaaaacaaaaaatatattagagttattccctctaccactaccccctacccaatattatagcttctga

SEQ ID NO: 32 - protein, 67 aa, Artificial Sequence
  CLIBASIA_03695 full length amino acid sequence
MSPLAPLGDGCEQLVGATVHSEGKKRSHDVPSSSNQEDQRGSPSPKKTKNILELFPLPLPPTQYYSF

SEQ ID NO: 33 - protein, 39 aa, Artificial Sequence
  CLIBASIA_03695 mature protein sequence
DVPSSSNQEDQRGSPSPKKTKNILELFPLPLPPTQYYSF

SEQ ID NO: 34 - DNA, 156 bp, Artificial Sequence
  CLIBASIA_03875 full length amino acid sequence
atggggtttcgtttttgggtatcacgtatacaaattttttttatcgctttgttgattgtattttctctctcttggtttgtttttactttgctttatttgattggtgatgttcgcttaaggacatgtgattttcaaatgacctttttttgtaagtaa

SEQ ID NO: 35 - protein, 51 aa, Artificial Sequence
  CLIBASIA_03875 full length amino acid sequence
MGFRFWVSRIQIFFIALLIVFSLSWFVFTLLYLIGDVRLRTCDFQMTFFCK

SEQ ID NO: 36 - protein, 24 aa, Artificial Sequence
  CLIBASIA_03875 mature protein sequence
FTLLYLIGDVRLRTCDFQMTFFCK

SEQ ID NO: 37 - DNA, 291 bp, Artificial Sequence
  CLIBASIA_04025 full length nucleotide sequence
atgacaatatcaaaaaatcaagccattcttttctttattacaggcatgatactttcttcttgtggtgatactctctctgactctaagcaacataataaaatcaacaatacaaaaaatcatcttgatcttcttttcccaatagacgactcccataatcaaaagcctacggaaaaaaaaccaaatacatcatccataaagataaagaacaatataatagaaccacaacccggccctagtcgctgggaaggaggatggaatggtgaaagatacgttagagaatgggaaagataa

SEQ ID NO: 38 - protein, 96 aa, Artificial Sequence
  CLIBASIA_04025 full length amino acid sequence
MTISKNQAILFFITGMILSSCGDTLSDSKQHNKINNTKNHLDLLFPIDDSHNQKPTEKKPNTSSIKIKNNIIEPQPGPSRWEGGWNGERYVREWER

SEQ ID NO: 39 - protein, 74 aa, Artificial Sequence
  CLIBASIA_04025 mature protein sequence
DTLSDSKQHNKINNTKNHLDLLFPIDDSHNQKPTEKKPNTSSIKIKNNIIEPQPGPSRWEGGWNGERYVREWER

SEQ ID NO: 40 - DNA, 480 bp, Artificial Sequence
  CLIBASIA_04040 full length nucleotide sequence
atgaaaagattgaaatatcaaattattttactctctcttttgagcaccaccatggcttcttgtggacaagctgatcctgtagctccaccaccaccacaaacattagcagaacgtggaaaagccttattagatgaagcaacgcaaaaagcagcagaaaaagcagcagaagcagcaagaaaagcagcagaacaagcagcagaagcagctaaaaaagcagcagaaaaaatcatacataaagacaagaagaaaccaaaggaaaaccaagaggtcaatgaggtaccggtagcagccaatatagagcccgaatctcaagaaacacaacaacaagtcatcaataagacaacaacatcacaaacagacgccgaaaaaacacctaacgaaaagcgtcaaggtacaacggatgggataaacaatcaatccaacgcaacaaatgatccatcatctaaagacaagatagcagaaaacacaaaagaggattag

SEQ ID NO: 41 - protein, 159 aa, Artificial Sequence
  CLIBASIA_04040 full length amino acid sequence
MKRLKYQIILLSLLSTTMASCGQADPVAPPPPQTLAERGKALLDEATQKAAEKAAEAARKAAEQAAEAAKKAAEKIIHKDKKKPKENQEVNEVPVAANIEPESQETQQQVINKTTTSQTDAEKTPNEKRQGTTDGINNQSNATNDPSSKDKIAENTKED

SEQ ID NO: 42 - protein, 135 aa, Artificial Sequence
  CLIBASIA_04040 mature protein sequence
DPVAPPPPQTLAERGKALLDEATQKAAEKAAEAARKAAEQAAEAAKKAAEKIIHKDKKKPKENQEVNEVPVAANIEPESQETQQQVINKTTTSQTDAEKTPNEKRQGTTDGINNQSNATNDPSSKDKIAENTKED

SEQ ID NO: 43 - DNA, 603 bp, Artificial Sequence
  CLIBASIA_04310 full length nucleotide sequence
atgaagaaaactgaaatattccgtatcttaaaagttatttggataggattgtatgtatccgtagcatcatttttcgtaactagtccaatttattccctctcacccgatcttataaaatatcatcaacaatcttctatgtctagtgatcttctcgatcaagaagaagtgaggactttgaaaatttatgtagtttccacgggatctaaagcgatcgttacttttaaacgtggtagccagtataatcaagaaggtttatcacaattaaatcgtttattgtatgattggcattccaaacaatctatagatatggatccacagttgtttgattttttatgggagatacaacaatatttcagtgtacccgaatatatttatatattatcaggatatcgtacgcaagaaaccaataaaatgttgagtaggcgaaatcgtaagatagctaggaaaagtcaacatgttttggggaaagctgttgatttttatattccaggagtttctttaaggagcctgtacaagatagctatacgtcttaaaagaggaggagttggttattattccaaatttcttcatattgatgtgggaagagtgcgttcttggacgtga

SEQ ID NO: 44 - protein, 200 aa, Artificial Sequence
  CLIBASIA_04310 full length amino acid sequence
MKKTEIFRILKVIWIGLYVSVASFFVTSPIYSLSPDLIKYHQQSSMSSDLLDQEEVRTLKIYVVSTGSKAIVTFKRGSQYNQEGLSQLNRLLYDWHSKQSIDMDPQLFDFLWEIQQYFSVPEYIYILSGYRTQETNKMLSRRNRKIARKSQHVLGKAVDFYIPGVSLRSLYKIAIRLKRGGVGYYSKFLHIDVGRVRSWT

SEQ ID NO: 45 - protein, 168 aa, Artificial Sequence
  CLIBASIA_04310 mature protein sequence
LSPDLIKYHQQSSMSSDLLDQEEVRTLKIYVVSTGSKAIVTFKRGSQYNQEGLSQLNRLLYDWHSKQSIDMDPQLFDFLWEIQQYFSVPEYIYILSGYRTQETNKMLSRRNRKIARKSQHVLGKAVDFYIPGVSLRSLYKIAIRLKRGGVGYYSKFLHIDVGRVRSWT

SEQ ID NO: 46 - DNA, 648 bp, Artificial Sequence
  CLIBASIA_04320 full length nucleotide
atggtacgtgttttttgtgcaataatctttgtattaattacgtttattggggaattttcccaagcgttagagcatgaggatgaattaaaggtcaactttggtctcatgcgacgagtaatgattgatttatggagccgtgaaatctcttcatatagaacacctctttctttagatttggattataaacacagggtttatctggatacatacaaaagctttagcataaatctcggatttgaaacatttaatgaaatagttaacccaactacgatgagagtgttggttctgcctgtgttatctatgcataaaacatggaataataattttgatgattcctatttttttaagaaaataggagtcggtgtcgtagcatctactgggttcaatacaggcgataagtggcttggagcagagatgggaatgtcattttacgtatacccgacaccgtggcttatattacagagcgattttgcgattcgtcacgccagtagcgatgttgttgtatgtatgcgttatcaagcgaaatttctgataactgatagtataggtattctttatcgaaatgtatcagcagtgtcagcggcagtagataagaatataggtttaggcgttactaagataggtcttgattatgtttataaattctaa

SEQ ID NO: 47 - protein, 215 aa, Artificial Sequence
  CLIBASIA_04320 full length amino acid sequence
MVRVFCAIIFVLITFIGEFSQALEHEDELKVNFGLMRRVMIDLWSREISSYRTPLSLDLDYKHRVYLDTYKSFSINLGFETFNEIVNPTTMRVLVLPVLSMHKTWNNNFDDSYFFKKIGVGVVASTGFNTGDKWLGAEMGMSFYVYPTPWLILQSDFAIRHASSDVVVCMRYQAKFLITDSIGILYRNVSAVSAAVDKNIGLGVTKIGLDYVYKF

SEQ ID NO: 48 - protein, 193 aa, Artificial Sequence
  CLIBASIA_04320 mature protein sequence
LEHEDELKVNFGLMRRVMIDLWSREISSYRTPLSLDLDYKHRVYLDTYKSFSINLGFETFNEIVNPTTMRVLVLPVLSMHKTWNNNFDDSYFFKKIGVGVVASTGFNTGDKWLGAEMGMSFYVYPTPWLILQSDFAIRHASSDVVVCMRYQAKFLITDSIGILYRNVSAVSAAVDKNIGLGVTKIGLDYVYKF

SEQ ID NO: 49 - DNA, 690 bp, Artificial Sequence
  CLIBASIA_04330 full length nucleotide sequence
atgaaatataagatcgcgattatcatattattggttttggttggagtgattttagctttttattttcaacagagcagtactccacaaaaccatcttctctttttttctgaaaaagcggtatggaaaggagattcggaaacatacttccaatgtaaacgagcacatgaattcgatgagaactttgacaactgtattatcacaagtattaaaaaaacgggaggaacccaagaagcgttacgagcggctcaatatctggaaaaatacttagagcctggatacgtatcttcatatcgtaaggaagctttaattggtatcgtagaggtgcaatatccttatagagctaatgagaatagcgggactttgcttattcctaccgtgggaagccatattattgatatcaatgattctagtgttcatcagctttacgattcttctcctatagcaaaggattttgtattaagaaatccaggtgttttcccttatagtgcgggacatttcgttaaaagcagtcataaagatggattaatagaattaattttttcttatcctttgagaagttgtcatggttgtgaagatattggttttatggatattgcatataaatttacaactaaaggtgcattcattggtagaaaagtatttggtattcggaatgataatgcaaaatatcctatgcacttctttatataa

SEQ ID NO: 50 - protein, 229 aa, Artificial Sequence
  CLIBASIA_04330 full length amino acid sequence
MKYKIAIIILLVLVGVILAFYFQQSSTPQNHLLFFSEKAVWKGDSETYFQCKRAHEFDENFDNCIITSIKKTGGTQEALRAAQYLEKYLEPGYVSSYRKEALIGIVEVQYPYRANENSGTLLIPTVGSHIIDINDSSVHQLYDSSPIAKDFVLRNPGVFPYSAGHFVKSSHKDGLIELIFSYPLRSCHGCEDIGFMDIAYKFTTKGAFIGRKVFGIRNDNAKYPMHFFI

SEQ ID NO: 51 - protein, 210 aa, Artificial Sequence
  CLIBASIA_04330 mature protein sequence
FYFQQSSTPQNHLLFFSEKAVWKGDSETYFQCKRAHEFDENFDNCIITSIKKTGGTQEALRAAQYLEKYLEPGYVSSYRKEALIGIVEVQYPYRANENSGTLLIPTVGSHIIDINDSSVHQLYDSSPIAKDFVLRNPGVFPYSAGHFVKSSHKDGLIELIFSYPLRSCHGCEDIGFMDIAYKFTTKGAFIGRKVFGIRNDNAKYPMHFFI

SEQ ID NO: 52 - DNA, 378 bp, Artificial Sequence
  CLIBASIA_04425 full length nucleotide sequence
atgaagaagtatatcacattattaacagtattactcataagtaacgtgctgaacctgtatgatgcgaaagcaagaagattcccaacctatggatctgaagaacgtatagcaacgtgcgcaaaaccgggctattcttcacgattagcacaactatgtgcagaaaacgaaaaaagacttaaagaattcgacaaaataacaagagaattgaatacgttatcagaaaacgaaaaaaaagcattctttgaacacgagaaaaaagtaacgagcaatctaaactacaacgcaagagacagaaagcataatataaatcaattctacgaagcgagaggaaagtaccgctacggaaatggatattatcgaaactaccgatcccagtaa

SEQ ID NO: 53 - protein, 125 aa, Artificial Sequence
  CLIBASIA_04425 full length amino acid sequence
MKKYITLLTVLLISNVLNLYDAKARRFPTYGSEERIATCAKPGYSSRLAQLCAENEKRLKEFDKITRELNTLSENEKKAFFEHEKKVTSNLNYNARDRKHNINQFYEARGKYRYGNGYYRNYRSQ

SEQ ID NO: 54 - protein, 106 aa, Artificial Sequence
  CLIBASIA_04425 mature protein sequence
YDAKARRFPTYGSEERIATCAKPGYSSRLAQLCAENEKRLKEFDKITRELNTLSENEKKAFFEHEKKVTSNLNYNARDRKHNINQFYEARGKYRYGNGYYRNYRSQ

SEQ ID NO: 55 - DNA, 588 bp, Artificial Sequence
  CLIBASIA_04560 full length nucleotide sequence
atgaaatcaaaaaatattctcattgtatcaacgttagtcatctgcgttctatctattagtagttgtgacctcggtgattccattgcaaaaaaaagaaatacaataggtaacacgatcaagaagtccataaatagagttatacaagagaataataaacctcgaaatatgactatatttaaaacagaagttaagagagatatacgtcgtgctagcaggctatctttggaagagaaatccaaaaatgcagataaacctactgtgatagagaatcaagctgataatatcaacattgaggtagaagtcgctactaatctgaacccaaaccatcaagctagtgagatcgatattgcaatagaaaacctgcctgatttgaaatcaaaccatcaagctagtgagatcgatattgcaatagaaaacctgcctgatttgaaatcaaaccatcaagctagtgagatcgatattgcaatagaaaacctgcctgatcatcaagttgatagaaatcataccctcagcaacctcagaggtgcttgttatcagccctctcttgtgtctaactcgtcgttaaagctatgggacgtagcattttaa

SEQ ID NO: 56 - protein, 195 aa, Artificial Sequence
  CLIBASIA_04560 full length amino acid sequence
MKSKNILIVSTLVICVLSISSCDLGDSIAKKRNTIGNTIKKSINRVIQENNKPRNMTIFKTEVKRDIRRASRLSLEEKSKNADKPTVIENQADNINIEVEVATNLNPNHQASEIDIAIENLPDLKSNHQASEIDIAIENLPDLKSNHQASEIDIAIENLPDHQVDRNHTLSNLRGACYQPSLVSNSSLKLWDVAF

SEQ ID NO: 57 - protein, 170 aa, Artificial Sequence
  CLIBASIA_04560 mature protein sequence
DSIAKKRNTIGNTIKKSINRVIQENNKPRNMTIFKTEVKRDIRRASRLSLEEKSKNADKPTVIENQADNINIEVEVATNLNPNHQASEIDIAIENLPDLKSNHQASEIDIAIENLPDLKSNHQASEIDIAIENLPDHQVDRNHTLSNLRGACYQPSLVSNSSLKLWDVAF

SEQ ID NO: 58 - DNA, 351 bp, Artificial Sequence
  CLIBASIA_04580 full length amino acid sequence
atgttttggattgcaaaaaaatttttttggatatcagtgttattaatcgttctgtctaatgtatatgcgcaaccttttttggaagagacggaaaaaggtaagaaaaccgaaatcacggattttatgactgccacaagtggtactgtgggttatgcgagcaatctttgtaatgcaaaaccagaaatatgtcttttgtggaaaaagattatgcgtaatgttaaaagacataccttaaatggagccaagattgtatatggttttgcgaaatcggctcttgagaaaaatgaaagagagagtgtagctatacattccaagaatgaatatccacctcctttgccgtcgcatcattag

SEQ ID NO: 59 - protein, 116 aa, Artificial Sequence
  CLIBASIA_04580 full length amino acid sequence
MFWIAKKFFWISVLLIVLSNVYAQPFLEETEKGKKTEITDFMTATSGTVGYASNLCNAKPEICLLWKKIMRNVKRHTLNGAKIVYGFAKSALEKNERESVAIHSKNEYPPPLPSHH

SEQ ID NO: 60 - protein, 93 aa, Artificial Sequence
  CLIBASIA_04580 mature protein sequence
QPFLEETEKGKKTEITDFMTATSGTVGYASNLCNAKPEICLLWKKIMRNVKRHTLNGAKIVYGFAKSALEKNERESVAIHSKNEYPPPLPSHH

SEQ ID NO: 61 - DNA, 489 bp, Artificial Sequence
  CLIBASIA_04735 full length nucleotide sequence
atgcatttttatcgttttattctcttaaatctttacatgctcacattattttcacatggctgtacacaaatagatttcggaaatatttttttcaaaaaaccagagatctcccttcctccttctgttgaatcagagattcttcttccgcctattcctgaagaagaatttgatcaggacgatatttctgtgcctagtaaggataataatgccattaggatgggaataataggtgcttggaaagtatcataccgagatgtcgactgtaagatgattttgacattgactcgatttaaaaagaattttcgtggaaccgctcgaagttgccatggtaggttagcatcattagcagcatggaatataatagatgaggatagttttgagcttaaaaataaatccggtcaaactatcattgttttctataaaactgcggaacagtctttcgagggatcttttcagggtgaaagtgataaagttataatttctcggtag

SEQ ID NO: 62 - protein, 162 aa, Artificial Sequence
  CLIBASIA_04735 full length amino sequence
MHFYRFILLNLYMLTLFSHGCTQIDFGNIFFKKPEISLPPSVESEILLPPIPEEEFDQDDISVPSKDNNAIRMGIIGAWKVSYRDVDCKMILTLTRFKKNFRGTARSCHGRLASLAAWNIIDEDSFELKNKSGQTIIVFYKTAEQSFEGSFQGESDKVIISR

SEQ ID NO: 63 - protein, 142 aa, Artificial Sequence
  CLIBASIA_04735 mature protein sequence
CTQIDFGNIFFKKPEISLPPSVESEILLPPIPEEEFDQDDISVPSKDNNAIRMGIIGAWKVSYRDVDCKMILTLTRFKKNFRGTARSCHGRLASLAAWNIIDEDSFELKNKSGQTIIVFYKTAEQSFEGSFQGESDKVIISR

SEQ ID NO: 64 - DNA, 618 bp, Artificial Sequence
  CLIBASIA_04870 full length nucleotide sequence
atgaatcgcgtatttgcgtatatattgttctgtttcctaggattgatggggtattctgttagtgcaagcaacaatgatacctctaaaataccagatgctaagtttggcagttttttacaaatcagatccaaagaatccgtcattaataaagagttcgtaaccaaggtggaagagctatacgaaaaagcccaaaaagcacataaaaaacgggataaggtatatggtgcttacgataaagtatcaagccataaaaaatcgcctaaagaattgagtaaagctttttacatagacttcagaacagaactaaaatattttaaagcactaactaaatattacaaatcagttgtagcagaactcagagaatttggtttaggcaaatccgcaatagaaattgaggaaatcactaaagccgtcgacacactcacaagagcgtataacgaatacaaaaaagaaataagagagttaatagaagagtttattgaattgggattcgaccagtgcgatgagtgtgatttgtgtagtgaaaaagcagatgtaatacaaaaaaaaaggatagcatttgaaatggtagaacgtgaattcgcggaaaaattagaaggtaaattcgtaagaaaataa

SEQ ID NO: 65 - protein, 205 aa, Artificial Sequence
  CLIBASIA_04870 full length amino acid sequence
MNRVFAYILFCFLGLMGYSVSASNNDTSKIPDAKFGSFLQIRSKESVINKEFVTKVEELYEKAQKAHKKRDKVYGAYDKVSSHKKSPKELSKAFYIDFRTELKYFKALTKYYKSVVAELREFGLGKSAIEIEEITKAVDTLTRAYNEYKKEIRELIEEFIELGFDQCDECDLCSEKADVIQKKRIAFEMVEREFAEKLEGKFVRK

SEQ ID NO: 66 - protein, 183 aa, Artificial Sequence
  CLIBASIA_04870 mature protein sequence
SNNDTSKIPDAKFGSFLQIRSKESVINKEFVTKVEELYEKAQKAHKKRDKVYGAYDKVSSHKKSPKELSKAFYIDFRTELKYFKALTKYYKSVVAELREFGLGKSAIEIEEITKAVDTLTRAYNEYKKEIRELIEEFIELGFDQCDECDLCSEKADVIQKKRIAFEMVEREFAEKLEGKFVRK

SEQ ID NO: 67 - DNA, 729 bp, Artificial Sequence
  CLIBASIA_04900 full length nucleotide sequence
atgaattttaatgggtatggtgcactttttttcgtagtatttttaagtattgtagtaccgaatcattcgttagcagtagatctttatctgcctaggaaaattgacttatttaatgaggcagataacaatgtggaatatcaggatgatgaatatggtatatggtctggtaattatgtaggattgcatatatctcgtttatatgaaacgcaccccttagctgatactatcaataggaaaacgtataatagtttactaccaaatggattgggaattgaattaggacataatatacagctagaagattttgtttttggtatcaattgtcatactactgctgcgaaggatgattctacgttttatcgtctaaaagaaaaatattttatttatggggatgttgtactaaaagcaggatattctgtggattctcttcttatctatggaatgggaggatttggaggagcatacgttatagattcaagccttgagaaggttgaatcagacaacagtaaaaatgctaaaggtagatttgatgggcatggatcaagtgtagtattaggtataggtttagattatatggtaaattacgacatctctttatctgctagttatcgttatattcctcatcacattcattctgttaataattctaacgcaaaaagtgatgttgaaagagtggacaggaaaggtaatgcccacatcgcatctcttggtataaatatgcacttctaa

SEQ ID NO: 68 - protein, 242 aa, Artificial Sequence
  CLIBASIA_04900 full length amino acid sequence
MNFNGYGALFFVVFLSIVVPNHSLAVDLYLPRKIDLFNEADNNVEYQDDEYGIWSGNYVGLHISRLYETHPLADTINRKTYNSLLPNGLGIELGHNIQLEDFVFGINCHTTAAKDDSTFYRLKEKYFIYGDVVLKAGYSVDSLLIYGMGGFGGAYVIDSSLEKVESDNSKNAKGRFDGHGSSVVLGIGLDYMVNYDISLSASYRYIPHHIHSVNNSNAKSDVERVDRKGNAHIASLGINMHF

SEQ ID NO: 69 - protein, 217 aa, Artificial Sequence
  CLIBASIA_04900 mature protein sequence
VDLYLPRKIDLFNEADNNVEYQDDEYGIWSGNYVGLHISRLYETHPLADTINRKTYNSLLPNGLGIELGHNIQLEDFVFGINCHTTAAKDDSTFYRLKEKYFIYGDVVLKAGYSVDSLLIYGMGGFGGAYVIDSSLEKVESDNSKNAKGRFDGHGSSVVLGIGLDYMVNYDISLSASYRYIPHHIHSVNNSNAKSDVERVDRKGNAHIASLGINMHF

SEQ ID NO: 70 - DNA, 558 bp, Artificial Sequence
  CLIBASIA_05115 full length nucleotide sequence
atgttcttaaatgttctaaaagatttttttgttcctaggatacgatttttgattgtattaatggtaagcagtgtatccgctgggtatgcgaatgcttcacaacctgagcctacattacgtaatcaattttccagatggtctgtatatgtatatccagatttaaataaaaaactttgtttttcactttctgttcctgttacggtagaaccgttagaaggtgttagacatggggttaatttctttattatttcattgaaaaaagaggaaaattctgcttatgtttcggaattagttatggattatcctttagatgaagaagagatggtttcgcttgaagtaaaaggaaaaaatgctagcggaacaatatttaaaatgaagtcttataataatagagctgcattcgaaaaaagatctcaagatactgttcttattgaggagatgaaacggggaaaagaattagttgtatccgccaaatctaaacgtggaacaaatacccgctatatctattctctcattggattatctgattctttggcagatattcgtaaatgtaattaa

SEQ ID NO: 71 - protein, 185 aa, Artificial Sequence
  CLIBASIA_05115 full length amino acid sequence
MFLNVLKDFFVPRIRFLIVLMVSSVSAGYANASQPEPTLRNQFSRWSVYVYPDLNKKLCFSLSVPVTVEPLEGVRHGVNFFIISLKKEENSAYVSELVMDYPLDEEEMVSLEVKGKNASGTIFKMKSYNNRAAFEKRSQDTVLIEEMKRGKELVVSAKSKRGTNTRYIYSLIGLSDSLADIRKCN

SEQ ID NO: 72 - protein, 153 aa, Artificial Sequence
  CLIBASIA_05115 mature protein sequence
SQPEPTLRNQFSRWSVYVYPDLNKKLCFSLSVPVTVEPLEGVRHGVNFFIISLKKEENSAYVSELVMDYPLDEEEMVSLEVKGKNASGTIFKMKSYNNRAAFEKRSQDTVLIEEMKRGKELVVSAKSKRGTNTRYIYSLIGLSDSLADIRKCN

SEQ ID NO: 73 - DNA, 681 bp, Artificial Sequence
  CLIBASIA_05150 full length nucleotide sequence
atgatgagagatataagaaaaattagaaattattttaggaatactgctaaaattatattgagtgggttatttctagggtttttttcttctgctgcaatggcagactatgggtattctccccagtttcagccgactataatggtgtccaattttgcaaaatttaaagggttatatgttgctgctgatttttccaaaatagatcatcagtcgcctgttcgtttgcaaaatctttctttaaatggggtgtccattggtcttgatggtcaagatggaacccttgtttatggtgcttctttgggtgtcgagggatttcatcttgaaccacgagggggaattgatggggataaggtagcgggaacactcttgtttcgtaccggttttacgtttgataataataattcttctattctccaaaatactcttatttatgggtttggtggagctcgtataagaaatattatgtctgttgaatctgctgacacagcaaaatccacaatacgaaacattgtagcaaacggttttttagataaagttattggtgtggggattgaaaagaaacttgctagcatgctctcgattcgtggtgagtatcgttatgtcgcttgttatgaccagccttgggatgtcagcaagtggagagaaaaaggtgacttcacagctggtgtggttttacgcttttaa

SEQ ID NO: 74 - protein, 226 aa, Artificial Sequence
  CLIBASIA_05150 full length amino acid sequence
MMRDIRKIRNYFRNTAKIILSGLFLGFFSSAAMADYGYSPQFQPTIMVSNFAKFKGLYVAADFSKIDHQSPVRLQNLSLNGVSIGLDGQDGTLVYGASLGVEGFHLEPRGGIDGDKVAGTLLFRTGFTFDNNNSSILQNTLIYGFGGARIRNIMSVESADTAKSTIRNIVANGFLDKVIGVGIEKKLASMLSIRGEYRYVACYDQPWDVSKWREKGDFTAGVVLRF

SEQ ID NO: 75 - protein, 192 aa, Artificial Sequence
  CLIBASIA_05150 mature protein sequence
DYGYSPQFQPTIMVSNFAKFKGLYVAADFSKIDHQSPVRLQNLSLNGVSIGLDGQDGTLVYGASLGVEGFHLEPRGGIDGDKVAGTLLFRTGFTFDNNNSSILQNTLIYGFGGARIRNIMSVESADTAKSTIRNIVANGFLDKVIGVGIEKKLASMLSIRGEYRYVACYDQPWDVSKWREKGDFTAGVVLRF

SEQ ID NO: 76 - DNA, 465 bp, Artificial Sequence
  CLIBASIA_05315 full length nucleotide sequence
gtgcgtaaaaatttattaacctcaacctcatctttaatgttttttttcttatcttctggctatgctttatctggcagtagttttggttgttgtggagaatttaaaaagaaagcttcttcacctagaatccatatgcgtcctttcaccaagtcatcaccttataacaactcagtgagtaatacagtgaataatactccgcgtgttcctgatgtctctgaaatgaacagctctaggggttctgctcctcaatctcatgttaatgtttcttctcctcattataaacatgaatacagttcttcttcggcatcttcttcaacacatgcttcgcctcctcctcattttgaacagaagcacattagtcgcactcgtattgactcaagccctccacccggtcatattgatcctcatcccgatcatattagaaatacacttgcactccatagaaaaatgttggagcagtcttga

SEQ ID NO: 77 - protein, 154 aa, Artificial Sequence
  CLIBASIA_05315 full length amino acid sequence
MRKNLLTSTSSLMFFFLSSGYALSGSSFGCCGEFKKKASSPRIHMRPFTKSSPYNNSVSNTVNNTPRVPDVSEMNSSRGSAPQSHVNVSSPHYKHEYSSSSASSSTHASPPPHFEQKHISRTRIDSSPPPGHIDPHPDHIRNTLALHRKMLEQS

SEQ ID NO: 78 - protein, 130 aa, Artificial Sequence
  CLIBASIA_05315 mature protein sequence
GSSFGCCGEFKKKASSPRIHMRPFTKSSPYNNSVSNTVNNTPRVPDVSEMNSSRGSAPQSHVNVSSPHYKHEYSSSSASSSTHASPPPHFEQKHISRTRIDSSPPPGHIDPHPDHIRNTLALHRKMLEQS

SEQ ID NO: 79 - DNA, 258 bp, Artificial Sequence
  CLIBASIA_05320 full length nucleotide sequence
atgagtaagtttgtggtgaggattatgtttttattaagtgctatatcttcgaatcctatcttagctgccaatgagcactcttctgtatcggaacagaagagaaaggagacaacagtaggatttatcagtcgtcttgtcaataaacgtcctgtcgctaataaacgttgtcctaatgcgactaaacaaacaccacccgatcatggatccaagtacgatacacgagaggtgcttatgctctttggaggcttaaacaattga

SEQ ID NO: 80 - protein, 85 aa, Artificial Sequence
  CLIBASIA_05320 full length amino acid sequence
MSKFVVRIMFLLSAISSNPILAANEHSSVSEQKRKETTVGFISRLVNKRPVANKRCPNATKQTPPDHGSKYDTREVLMLFGGLNN

SEQ ID NO: 81 - protein, 63 aa, Artificial Sequence
  CLIBASIA_05320 mature protein sequence
ANEHSSVSEQKRKETTVGFISRLVNKRPVANKRCPNATKQTPPDHGSKYDTREVLMLFGGLNN

SEQ ID NO: 82 - DNA, 207 bp, Artificial Sequence
  CLIBASIA_05640 full length nucleotide sequence
atgactattaagaaagtactaattgcttcaactttattatccctctgtggctgtggtttagcggatgaaccaaagaagctgaatcctgatcaactctgtgatgccgtttgtaggcttactttagaagaacaaaaagagttacaaactaaggtaaatcagaggtatgaagaacaccttacaaagggtgcgaaactatctagtgattaa

SEQ ID NO: 83 - protein, 68 aa, Artificial Sequence
  CLIBASIA_05640 full length amino acid sequence
MTIKKVLIASTLLSLCGCGLADEPKKLNPDQLCDAVCRLTLEEQKELQTKVNQRYEEHLTKGAKLSSD

SEQ ID NO: 84 - protein, 47 aa, Artificial Sequence
  CLIBASIA_05640 mature protein sequence
DEPKKLNPDQLCDAVCRLTLEEQKELQTKVNQRYEEHLTKGAKLSSD

SEQ ID NO: 85 - DNA, 22 bp, Artificial Sequence
  CLIBASIA_00420 reverse transciption primer
ctggatccatctgtagagatgg

SEQ ID NO: 86 - DNA, 30 bp, Artificial Sequence
  CLIBASIA_00420 forward primer
cggtgctttttatccttatcgttccttttt

SEQ ID NO: 87 - DNA, 30 bp, Artificial Sequence
  CLIBASIA_00420 reverse primer
ggcaataactcactgctatcaacaacgact

SEQ ID NO: 88 - DNA, 27 bp, Artificial Sequence
  CLIBASIA_00460 reverse transciption primer
ataaaagttataggttcacctcccata

SEQ ID NO: 89 - DNA, 21 bp, Artificial Sequence
  CLIBASIA_00460 forward primer
gcctcgtattgcaacaaaatc

SEQ ID NO: 90 - DNA, 19 bp, Artificial Sequence
  CLIBASIA_00460 reverse primer
acggcggaagaagatgaag

SEQ ID NO: 91 - DNA, 17 bp, Artificial Sequence
  CLIBASIA_00525 reverse transciption primer
acaattttctgtatcgc

SEQ ID NO: 92 - DNA, 30 bp, Artificial Sequence
  CLIBASIA_00525 forward primer
ttataaatcaatgcatccgtctacgcaaga

SEQ ID NO: 93 - DNA, 30 bp, Artificial Sequence
  CLIBASIA_00525 reverse primer
tatttcggcttctgtcctttttccagttta

SEQ ID NO: 94 - DNA, 20 bp, Artificial Sequence
  CLIBASIA_00530 reverse transciption primer
tggaccatccatacgtctca

SEQ ID NO: 95 - DNA, 20 bp, Artificial Sequence
  CLIBASIA_00530 forward primer
ctgtgccgatgacaggatta

SEQ ID NO: 96 - DNA, 20 bp, Artificial Sequence
  CLIBASIA_00530 reverse primer
agccgggtacaattcctttt

SEQ ID NO: 97 - DNA, 19 bp, Artificial Sequence
  CLIBASIA_01640 reverse transciption primer
ctcatgttgccctgtatcg

SEQ ID NO: 98 - DNA, 30 bp, Artificial Sequence
  CLIBASIA_01640 forward primer
gttgctgtagggtatcctgctgtaaaaggt

SEQ ID NO: 99 - DNA, 30 bp, Artificial Sequence
  CLIBASIA_01640 reverse primer
ccaactctcaaataaccacgcgtataacaa

SEQ ID NO: 100 - DNA, 19 bp, Artificial Sequence
  CLIBASIA_02470 reverse transcription primer
gctccagaagcagcaaagg

SEQ ID NO: 101 - DNA, 30 bp, Artificial Sequence
  CLIBASIA_02470 forward primer
aatcaatggctcttaattgcaacgaaactt

SEQ ID NO: 102 - DNA, 30 bp, Artificial Sequence
  CLIBASIA_02470 reverse primer
tatatttcccatgccatttgtgatttttca

SEQ ID NO: 103 - DNA, 20 bp, Artificial Sequence
  CLIBASIA_03695 reverse transciption primer
ggcttcccctttgatcttct

SEQ ID NO: 104 - DNA, 20 bp, Artificial Sequence
  CLIBASIA_03695 forward primer
aagcgccatcctaccctact

SEQ ID NO: 105 - DNA, 20 bp, Artificial Sequence
  CLIBASIA_03695 reverse primer
gggcacatcgtgacttcttt

SEQ ID NO: 106 - DNA, 20 bp, Artificial Sequence
  CLIBASIA_03875 reverse transcription primer
tccttaagcgaacatcacca

SEQ ID NO: 107 - DNA, 20 bp, Artificial Sequence
  CLIBASIA_03875 forward primer
tttcgtttttgggtatcacg

SEQ ID NO: 108 - DNA, 24 bp, Artificial Sequence
  CLIBASIA_03875 reverse primer
gcaaagtaaaaacaaaccaagaga

SEQ ID NO: 109 - DNA, 20 bp, Artificial Sequence
  CLIBASIA_04025 reverse transcription primer
atctttcccattctctaacg

SEQ ID NO: 110 - DNA, 30 bp, Artificial Sequence
  CLIBASIA_04025 forward primer
tcttcttttcccaatagacgactcccataa

SEQ ID NO: 111 - DNA, 30 bp, Artificial Sequence
  CLIBASIA_04025 reverse primer
cccattctctaacgtatctttcaccattcc

SEQ ID NO: 112 - DNA, 20 bp, Artificial Sequence
  CLIBASIA_04310 reverse transciption primer
cgtccaagaacgcactcttc

SEQ ID NO: 113 - DNA, 30 bp, Artificial Sequence
  CLIBASIA_04310 forward primer
catgttttggggaaagctgttgatttttat

SEQ ID NO: 114 - DNA, 30 bp, Artificial Sequence
  CLIBASIA_04310 reverse primer
tcttcccacatcaatatgaagaaatttgga

SEQ ID NO: 115 - DNA, 19 bp, Artificial Sequence
  CLIBASIA_04330 reverse transription primer
gtaacgcttcttgggttcc

SEQ ID NO: 116 - DNA, 30 bp, Artificial Sequence
  CLIBASIA_04330 forward primer
cagagcagtactccacaaaaccatcttctc

SEQ ID NO: 117 - DNA, 30 bp, Artificial Sequence
  CLIBASIA_04330 reverse primer
agttctcatcgaattcatgtgctcgtttac

SEQ ID NO: 118 - DNA, 20 bp, Artificial Sequence
  CLIBASIA_04425 reverse transciption primer
tccgtagcggtactttcctc

SEQ ID NO: 119 - DNA, 25 bp, Artificial Sequence
  CLIBASIA_04425 forward primer
ctcataagtaacgtgctgaacctgt

SEQ ID NO: 120 - DNA, 25 bp, Artificial Sequence
  CLIBASIA_04425 reverse primer
gttgctatacgttcttcagatccat

SEQ ID NO: 121 - DNA, 20 bp, Artificial Sequence
  CLIBASIA_04560 reverse transciption primer
gggctgataacaagcacctc

SEQ ID NO: 122 - DNA, 21 bp, Artificial Sequence
  CLIBASIA_04560 forward primer
tgctagcaggctatctttgga

SEQ ID NO: 123 - DNA, 24 bp, Artificial Sequence
  CLIBASIA_04560 reverse primer
gcgacttctacctcaatgttgata

SEQ ID NO: 124 - DNA, 18 bp, Artificial Sequence
  CLIBASIA_04580 reverse transcription primer
ctcaagagccgatttcgc

SEQ ID NO: 125 - DNA, 30 bp, Artificial Sequence
  CLIBASIA_04580 forward primer
tcgttctgtctaatgtatatgcgcaacctt

SEQ ID NO: 126 - DNA, 30 bp, Artificial Sequence
  CLIBASIA_04580 reverse primer
gcaaaaccatatacaatcttggctccattt

SEQ ID NO: 127 - DNA, 21 bp, Artificial Sequence
  CLIBASIA_04735 reverse transcription primer
gtcaaaatcatcttacagtcg

SEQ ID NO: 128 - DNA, 30 bp, Artificial Sequence
  CLIBASIA_04735 forward primer
cttcttccgcctattcctgaagaagaattt

SEQ ID NO: 129 - DNA, 30 bp, Artificial Sequence
  CLIBASIA_04735 reverse primer
ttccaagcacctattattcccatcctaatg

SEQ ID NO: 130 - DNA, 20 bp, Artificial Sequence
  CLIBASIA_04800 reverse transcription primer
tgtagctccttctgcacgtc

SEQ ID NO: 131 - DNA, 20 bp, Artificial Sequence
  CLIBASIA_04800 forward primer
tcgcagtagctgatttcgtg

SEQ ID NO: 132 - DNA, 20 bp, Artificial Sequence
  CLIBASIA_04800 reverse primer
tacattcctcagcggctttt

SEQ ID NO: 133 - DNA, 20 bp, Artificial Sequence
  CLIBASIA_05150 reverse transcription primer
cgaatcgagagcatgctagc

SEQ ID NO: 134 - DNA, 30 bp, Artificial Sequence
  CLIBASIA_05150 forward primer
acactcttgtttcgtaccggttttacgttt

SEQ ID NO: 135 - DNA, 30 bp, Artificial Sequence
  CLIBASIA_05150 reverse primer
aaaccgtttgctacaatgtttcgtattgtg

SEQ ID NO: 136 - DNA, 19 bp, Artificial Sequence
  CLIBASIA_05315 reverse transcription primer
cgggtggagggcttgagtc

SEQ ID NO: 137 - DNA, 30 bp, Artificial Sequence
  CLIBASIA_05315 forward primer
cctgatgtctctgaaatgaacagctctagg

SEQ ID NO: 138 - DNA, 30 bp, Artificial Sequence
  CLIBASIA_05315 reverse primer
gagtgcgactaatgtgcttctgttcaaaat

SEQ ID NO: 139 - DNA, 20 bp, Artificial Sequence
  CLIBASIA_05310 reverse transcription primers
atgatcgggtggtgtttgtt

SEQ ID NO: 140 - DNA, 20 bp, Artificial Sequence
  CLIBASIA_05320 forward primer
tcttagctgccaatgagcac

SEQ ID NO: 141 - DNA, 20 bp, Artificial Sequence
  CLIBASIA_05320 reverse primer
agcgacaggacgtttattga

SEQ ID NO: 142 - DNA, 20 bp, Artificial Sequence
  CLIBASIA_05640 reverse transcription primer
tcactagatagtttcgcacc

SEQ ID NO: 143 - DNA, 30 bp, Artificial Sequence
  CLIBASIA_05640 forward primer
gcttcaactttattatccctctgtggctgt

SEQ ID NO: 144 - DNA, 30 bp, Artificial Sequence
  CLIBASIA_05640 reverse primer
gatagtttcgcaccctttgtaaggtgttct

SEQ ID NO: 145 - DNA, 29 bp, Artificial Sequence
  16s Las long reverse transciption primer
tccctataaagtacccaacactaggtaaa

SEQ ID NO: 146 - DNA, 26 bp, Artificial Sequence
  16s las long forward primer
cttaccagcccttgacatgtatagga

SEQ ID NO: 147 - DNA, 29 bp, Artificial Sequence
  16s las long reverse primer
tccctataaagtacccaacactaggtaaa

SEQ ID NO: 148 - DNA, 20 bp, Artificial Sequence
  UPL7 reverse transciption primer
tcaggaacagcaaaagcaag

SEQ ID NO: 149 - DNA, 20 bp, Artificial Sequence
  UPL7 forward primer
caaagaagtgcagcgagaga

SEQ ID NO: 150 - DNA, 20 bp, Artificial Sequence
  UPL7 reverse primer
tcaggaacagcaaaagcaag

(19) United States
(12) Patent Application Publication
(10) Pub. No.: US 2021/0116451 A1
(43) Pub. Date: Apr. 22, 2021

(54) PATHOGEN EFFECTORS FOR EARLY DETECTION OF CITRUS GREENING DISEASE

(71) Applicants: University of Florida Research Foundation, Inc., Gainesville, FL (US); The United States of America as Represented by The Secretary of Agriculture, Washington, DC (US)

(72) Inventors: Liliana Maria Cano Mogrovejo, Port St. Lucie, FL (US); Marco Pitino, Port St. Lucie, FL (US); Qingchun Shi, New Haven, CT (US); Ed Stover, Fort Pierce, FL (US); Robert G. Shatters, JR., Fort Pierce, FL (US)

(21) Appl. No.: 16/864,996

(22) Filed: May 1, 2020

Related U.S. Application Data
(60) Provisional application No. 62/841,616, filed on May 1, 2019.

(51) Int. Cl. G01N 33/569

(52) U.S. Cl. CPC G01N 33/56911; G01N 2333/195

(57) ABSTRACT

Disclosed herein methods and kits for early detection of huanglongbing (HLB) in a subject at risk for contracting HLB.

### CROSS-REFERENCE TO RELATED APPLICATION

This application claims priority to U.S. Provisional Application entitled “PATHOGEN EFFECTORS FOR EARLY DETECTION OF CITRUS GREENING DISEASE,” having Ser. No. 62/841,616, filed on May 1, 2019, which is entirely incorporated herein by reference.

### FEDERAL SPONSORSHIP

This invention was made with government support under Cooperative Agreement 58-6034-8-014 awarded by The United States Department of Agriculture, Agricultural Research Service. The government has certain rights in the invention.

### SEQUENCE LISTING

This application contains a sequence listing filed in electronic form as an ASCII.txt file entitled “222110_1065_Sequence_Listing_ST25.txt”, created on Dec. 28, 2020 and having a size of 100 kb. The content of the sequence listing is incorporated herein in its entirety.

### BACKGROUND

Citrus huanglongbing (HLB), also known as citrus greening, is a devastating disease with high economical costs to the worldwide citrus industry. The disease is caused by three species of alpha-proteobacterium, “Candidatus Liberibacter asiaticus (Las),” “Ca. L. africanus,” and “Ca. L. americanus.” Las, the most widespread pathogen, is vectored by the Asian Citrus Psyllid (ACP) Diaphorina citri Kuwayama (Hemiptera: Psyllidae). Las attacks all species and hybrids in the Citrus genus, and upon infection, resides in the phloem of the host causing a systemic disease and can eventually kill the tree. Once a tree is infected, it is extremely difficult to cure, and currently there is no adequate strategy for HLB management.

A potential strategy for detection, in particular early detection, of HLB in citrus would be detection of biological components (proteins, sugars, nucleic acids, and such) of the Las bacteria upon infection of the host. In 2009, the complete genome sequence of Las was obtained, enabling heterologous expression of Las proteins to be performed. Unfortunately, even with the examination of citrus transcriptomes in response to Las infection and comparative transcriptome analyses, Las genes involved in psyllid or citrus colonization remain largely unknown. This lack of accomplishment is due in part to the limited success in culturing the bacterium and the large number of hypothetical proteins in the sequenced Las genome, combined with a limited understanding of the function of putative proteins within the sequenced Las genome.

Although previously demonstrated assays showed promise as high-throughput and economic approaches for HLB diagnosis, such assays were preliminary and additional validation, evaluation, and most importantly, development of an optimized sampling protocol for a large number of samples (e.g., citrus varieties, tree ages, geographic locations, etc.) is required before this technology can be incorporated in the suite of HLB diagnostic tools.

Accordingly, there is a need to address the aforementioned deficiencies and inadequacies, in particular to characterize proteins transcribed and translated from the genome by the bacterium.

### SUMMARY

Described herein are kits and methods relating to detection of citrus huanglongbing (HLB).

In embodiments according to the present disclosure, described herein are kits for detection of citrus huanglongbing (HLB) in a subject in need thereof. Kits as described herein can comprise two or more antibodies, wherein the two or more antibodies are configured to recognize different amino acid sequences encoding effector proteins from Candidatus Liberibacter asiaticus selected from the group consisting of: SEQ ID Nos.:5, 6, 32, 33, 38, 39, 59, 60, 77, 78, 80, 81, 2, 3, 11, 12, 35, 36, 50, 51, 53, 54, 83, 84, or substantially identical variants thereof.

One of the antibodies of the kits as described herein recognizes SEQ ID Nos.:32, 33, or substantially identical variants thereof.

Kits as described herein can be configured as a microtiter plate-based assay. Kits as described herein can be configured as an enzyme-linked immunosorbent assay (ELISA). Kits as described herein can be configured as a spot-ELISA.

Antibodies of kits as described herein can be detectably labeled.

The subject in need thereof can be an infected citrus plant or psyllid insect or citrus plant or psyllid insect at risk for infection.

Additional embodiments of kits for detection of citrus huanglongbing (HLB) in a subject in need thereof comprise one or more antibodies, wherein the one or more antibodies are configured to recognize at least an amino acid sequence encoding an effector protein from Candidatus Liberibacter asiaticus of SEQ ID NO:32, 33, or substantially identical variants thereof. Embodiments of kits according to the present disclosure further comprise one or more antibodies configured to recognize SEQ ID Nos.:5, 6, 38, 39, 59, 60, 77, 78, 80, 81, 2, 3, 11, 12, 35, 36, 50, 51, 53, 54, 83, 84, or substantially identical variants thereof.

Also described herein is a kit for detection of citrus huanglongbing (HLB) in a subject in need thereof, comprising one or more antibodies, wherein the one or more antibodies are configured to recognize at least an amino acid sequence encoding an effector protein from Candidatus Liberibacter asiaticus of SEQ ID NO:32, 33, or substantially identical variants thereof. The kit further comprises one or more antibodies configured to recognize SEQ ID Nos.:5, 6, 38, 39, 59, 60, 77, 78, 80, 81, 2, 3, 11, 12, 35, 36, 50, 51, 53, 54, 83, 84, or substantially identical variants thereof.

Kits as described herein can be configured as a microtiter plate-based assay.

Further described herein are methods for detection of citrus huanglongbing (HLB) in a subject in need thereof. Embodiments of methods as described herein comprise providing a sample from the subject; and administering the sample to a kit as described herein. The subject in need thereof can be an infected citrus plant or psyllid insect, or citrus plant or psyllid insect at risk for infection. The sample can be sap or a leaf. The sap or leaf can be from a plant selected from the group consisting of individuals or hybrids of: C. aurantium, C. tangerine, C. ichangensis, C. limetta, C. unshiu, C. maxima, C. grandis, C. Paradisi, C. maxima, C. limon, C. japonica, C. glauca, C. bergamia, C. sinensis. C. reticulata, Poncirus trifoliate.

Further described herein are embodiments of methods for detection of citrus huanglongbing (HLB) in a subject in need thereof. Methods as described herein comprise providing a sample; extracting proteins from the sample; administering two or more antibodies to the extracted proteins, wherein the two or more antibodies are configured to recognize different amino acid sequences encoding effector proteins from Candidatus Liberibacter asiaticus selected from the group consisting of: SEQ ID Nos.:5, 6, 32, 33, 38, 39, 59, 60, 77, 78, 80, 81, 2, 3, 11, 12, 35, 36, 50, 51, 53, 54, 83, 84, or substantially identical variants thereof; forming a plurality of immunoconjugates by incubating the mixture of extracted proteins and two or more antibodies; detecting the immunoconjugates; determining from the detected immunoconjugates a disease status; and outputting the disease status.

One of the antibodies of methods as described herein recognizes SEQ ID Nos.:32, 33, or substantially identical variants thereof. Antibodies as described herein are detectably labeled.

The subject in need thereof is an infected citrus plant or psyllid insect or citrus plant or psyllid insect at risk for infection.

In an embodiment, a method for detection of citrus huanglongbing (HLB) in a subject in need thereof comprises providing a sample; extracting proteins from the sample; administering one or more antibodies to the extracted proteins, wherein the one or more antibodies are configured to recognize at least an amino acid sequence encoding an effector protein from Candidatus Liberibacter asiaticus of SEQ ID NO:32, 33, or substantially identical variants thereof; forming a plurality of immunoconjugates by incubating the mixture of extracted proteins and one or more antibodies; detecting the immunoconjugates; determining from the detected immunoconjugates a disease status; and outputting the disease status

The method can further comprise administering one or more antibodies configured to recognize SEQ ID Nos.:5, 6, 38, 39, 59, 60, 77, 78, 80, 81, 2, 3, 11, 12, 35, 36, 50, 51, 53, 54, 83, 84, or substantially identical variants thereof.

The one or more antibodies can be detectably labeled. The subject in need thereof is an infected citrus plant or psyllid insect or citrus plant or psyllid insect at risk for infection.

Kits as described herein can comprise two or more forward and reverse primer pairs for generation of amplicons relating to sequences as described herein. Kits can further comprise one or more primers for reverse transcription. Kits can comprise one or more primers of SEQ ID NOs:85-150.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1C. Steps for the study on expression of Ca. L. asiaticus effectors in citrus hosts according to the present disclosure. FIG. 1A illustrates a bioinformatics pipeline that was applied to identify secreted candidate effectors using 1,136 Coding Gene Sequences CDS from Ca. L. asiaticus genome. FIG. 1B represents preliminary detection of mRNA using Real Time quantitative Polymerase Chain Reaction RT-qPCR measured expression of 20 candidate effector genes in infected citrus. FIG. 1C illustrates effector expression pattern analysis during early and late bacterial-host interaction, in different citrus genotypes and between different tissue types.

FIGS. 2A-2D. FIG. 2A shows photographs of detached leaf inoculation using Ca. L. asiaticus-infected ACP and the distribution of bacterial titer measured from leaf samples collected at 6 hours, 1, 3 and 7 days post ACP exposure (FIGS. 2B-2D). The experiments were conducted using leaves from citron (FIG. 2B), Duncan (FIG. 2C) and Cleopatra (FIG. 2D). The bacterium was quantified by qPCR amplifying the 16s rDNA with Las long primer from 100 ng of citrus DNA template. The cycle threshold values (Ct) were used to generated the dot plots for each time point.

FIGS. 3A-3C are graphs illustrating a linear relationship between the numbers of effectors detected by RT-qPCR and Ca. L. asiaticus titer in citron (FIG. 3A), Duncan (FIG. 3B) and Cleopatra (FIG. 3C). The calculation used samples from the detached leaf assay collected at 6 hours, 1, 3 and 7 days post ACP exposure, and from leaf samples collected at 2, 4, 6, 8 and 10 weeks after ACP inoculation to the citrus plants (Shi et al, 2017). The data analysis was done by JMP Genomics Fit Y by X function.

FIGS. 4A-4F are graphs illustrating the expression of Ca. L. asiaticus effectors in Duncan (FIG. 4A), Washington navel (FIG. 4B), citron (FIG. 4C), Cleopatra (FIG. 4D), Pomeroy (FIG. 4E), and Carrizo (FIG. 4F) after 7-day ACP infestation in a detached leaf assay. Three random selected Ca. L. asiaticus positive leaves were used for RNA isolation and expression analysis. The Ct values of each effector generated by RT-qPCR were normalized with citrus endogenous control UPL7 and transformed into relative quantification (RQ) values (2^{ΔCt}). The effectors with expression detections were subject to pair-wise comparison of standard least-square means (LS means) with Student's t-test (p<0.05). Expression levels indicated with the same letter are not significantly different. Bars are means±standard error (n=3).

FIGS. 5A-5E Comparison of effector expression in the leaf and root of citron (FIG. 5A), Duncan (FIG. 5B) and Cleopatra (FIG. 5C) of healthy plants (CLas−) and plants infected by Ca. L. asiaticus (CLas+). Photographs display the health of Ca. L. asiaticus infected plants (FIGS. 5A-5C) over a year after inoculation (right frames, CLas+), in comparison to their uninfected controls (left frames, CLas−). The Ca. L. asiaticus was quantified by qPCR amplifying 16s rDNA from 100 ng of DNA template. A standard curve method with the function Log (copy number)=−0.289*Ct+11.66 was used to calculate 16s copy numbers (FIG. 5D). The RNA was isolated from the same group of samples for effector analysis by RT-qPCR, from which the number of effectors detected was compared between leaf and root tissues in citron, Duncan and Cleopatra (FIG. 5E). Analysis was based on three biological replicates and was analyzed by Student's t-test (p<0.05) and significant difference between leaf/root pairs is indicated by an asterisk.

FIGS. 6A-6F Comparison of effector expression in the leaf and root tissues of citron (FIGS. 6A and 6B, respectively), Duncan (FIGS. 6C and 6D, respectively) and Cleopatra (FIGS. 6E and 6F, respectively). Leaf and root samples were collected from three plants of each citrus genotype (replicates) that were inoculated 14-17 months prior via ACP infestation. In the expression analysis, the Ct values of each effector generated by RT-qPCR were normalized with citrus endogenous control UPL7 and transformed into relative quantification (RQ) values (2^{ΔCt}). The effectors with expression detected were subject to pair-wise comparison of standard least-square means (LS means) with Student's t-test (p<0.05). Expression levels indicated with the same letter are not significantly different. Bars are means±standard error (n=3).

FIGS. 7A-7C show Successful in planta transient expression, using agroinfiltration, of CLIBASIA_4580 in Nicotiana benthamiana was assessed by observing the presence or absence of CLIBASIA_4580 protein and detected by western blotting using a rabbit polyclonal CLIBASIA_4580-HRP antibody. The antibodies were conjugated to enzymes horseradish peroxidase (HRP) eliminating the need for the secondary antibody incubation steps. At 1 dpi (day post agroinfiltration) total proteins were extracted from N. benthamiana infiltrated leaves and subjected to western blotting. Transient expression of CLIBASIA_5315 was used as negative control.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, microbiology, molecular biology, plant pathology, horticulture, botany, bacteriology, and the like.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

“Subject” as used herein denotes a plant or insect susceptible or otherwise at risk for bacterial infection. In certain aspects, the bacterial infection can be an infection by the Candidatus Liberibacter asiaticus bacteria (also referred to herein as CLas, Las, C. Las, Ca. L. asiaticus, and so forth). The bacterial infection can be Citrus Huanglongbing (HLB) disease (also referred to herein as citrus greening disease).

“Sample” as used herein, sample can refer to a part of the subject at risk for bacterial infection, for example, part of a leaf or root of a plant or a component thereof.

As used herein, “control” is an alternative subject or sample used in an experiment for comparison purposes and included to minimize or distinguish the effect of variables other than an independent variable.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid sequence” and “oligonucleotide” also encompasses a nucleic acid and polynucleotide as defined above.

As used herein, “DNA molecule” includes nucleic acids/polynucleotides that are made of DNA.

As used herein, “wild-type” is the typical form of an organism, variety, strain, gene, protein, or characteristic as it occurs in nature, as distinguished from mutant forms that may result from selective breeding or transformation with a transgene.

As used herein, “identity,” is a relationship between two or more polypeptide or polynucleotide sequences, as determined by comparing the sequences. In the art, “identity” also refers to the degree of sequence relatedness between polypeptide as determined by the match between strings of such sequences. “Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch (J. Mol. Biol., 1970, 48: 443-453) algorithm (e.g., NBLAST, and XBLAST). The default parameters are used to determine the identity for the polypeptides or polynucleotides of the present disclosure.

As used herein, “heterologous” refers to compounds, molecules, nucleotide sequences (including genes), and polypeptide sequences (including peptides and proteins) that are different in both activity (function) and sequence or chemical structure. As used herein, “heterologous” can also refer to a gene or gene product that is from a different organism. For example, a human GTP cyclohydrolase or a synthase can be said to be heterologous when expressed in yeast.

As used herein, “homologue” refers to a polypeptide sequence that shares a threshold level of similarity and/or identity as determined by alignment of matching amino acids. Two or more polypeptides determined to be homologues are said to be homologues. Homology is a qualitative term that describes the relationship between polypeptide sequences that is based upon the quantitative similarity.

As used herein, “paralog” refers to a homologue produced via gene duplication of a gene. In other words, paralogs are homologues that result from divergent evolution from a common ancestral gene.

As used herein, “orthologues” refers to homologues produced by speciation followed by divergence of sequence but not activity in separate species. When speciation follows duplication and one homologue sorts with one species and the other copy sorts with the other species, subsequent divergence of the duplicated sequence is associated with one or the other species. Such species specific homologues are referred to herein as orthologues.

As used herein, “xenologs” are homologues resulting from horizontal gene transfer.

As used herein, “similarity” is a quantitative term that defines the degree of sequence match between two compared polypeptide sequences.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “culturing” refers to maintaining cells under conditions in which they can proliferate and avoid senescence as a group of cells. “Culturing” can also include conditions in which the cells also or alternatively differentiate.

As used herein, “gene” refers to a hereditary unit corresponding to a sequence of DNA that occupies a specific location on a chromosome and that contains the genetic instruction for a characteristic(s) or trait(s) in an organism. As used herein, “synthetic gene” can refer to a recombinant gene comprising one or more coding sequences for a protein of interest, or a synthetically purified protein that is not naturally occurring in its purified state.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “cDNA” refers to a DNA sequence that is complementary to a RNA transcript in a cell. It is a non-naturally occurring man-made molecule. Typically, cDNA is made in vitro by an enzyme called reverse-transcriptase using RNA transcripts as templates.

As used herein “chemical” refers to any molecule, compound, particle, or other substance that can be a substrate for an enzyme in the enzymatic pathway described herein and/or a carboxylesterase enzyme or biochemical pathway. A “chemical” can also be used to refer to a metabolite of a carboxylic ester. As such, “chemical” can refer to nucleic acids, proteins, organic compounds, inorganic compounds, metabolites etc.

As used herein “biologically coupled” refers to the association of or interaction between two or more physically distinct molecules, groups of molecules compounds, organisms, or particles where the association is directly or indirectly mediated between the two or more physically distinct molecules, groups of molecules compounds, organisms or particles via a biologic molecule or compound. This can include direct binding between two biologic molecules and signal transduction pathways.

As used herein, “biological communication” refers to the communication between two or more molecules, compounds, or objects that is mediated by a biologic molecule or biologic interaction.

As used herein, “biologic molecule,” “biomolecule,” “biological target” and the like refer to any molecule that is present in a living organism and includes without limitation, macromolecules (e.g. proteins, polysaccharides, lipids, and nucleic acids) as well as small molecules (e.g. metabolites and other products produced by a living organism).

As used herein, “regulation” refers to the control of gene or protein expression or function.

As used herein, “native” refers to the endogenous version of a molecule or compound relative to the host cell or population being described.

As used herein, “non-naturally occurring” refers to a non-native version of a molecule or compound or non-native expression or presence of a molecule or compound within a host cell or other composition. This can include where a native molecule or compound is influenced to be expressed or present at a different location within a host, at a non-native period of time within a host, or is otherwise in an altered environment, even when considered within the host. Non-limiting examples include where a protein that is expressed only in the nucleus of a cell is expressed in the cytoplasm of the cell or when a protein that is only normally expressed during the embryonic stage of development is expressed during the adult stage.

As used herein, “encode” refers to the biologic phenomena of transcribing DNA into an RNA that, in some cases, can be translated into a protein product. As such, when a protein is said herein to be encoded by a particular nucleotide sequence, it is to be understood that this refers to this biologic relationship between DNA and protein. It is well established that RNA can be translated into protein based on the triplet code where 3 nucleotides represent an amino acid. This term also includes the idea that DNA can be transcribed into RNA molecules with biologic functions, such as ribozymes and interfering RNA species. As such, when a RNA molecule is said to be encoded by a particular nucleotide sequence it is to be understood that this is referring to the transcriptional relationship between the DNA and RNA species in question. As such “encoding nucleotide” refers to herein as the nucleotide which can give rise through transcription, and in the case of proteins, translation a functional RNA or protein.

As described herein, the phrase “donor plant” refers to the plant to which the genetic modifications according to the present disclosure are performed to produce the desired outcome or phenotype.

A “native gene” or “an endogenous gene” is a gene that is naturally found in a host microorganism; whereas, an “exogenous gene” is a gene introduced into a host microorganism and which was obtained from a microorganism other the host microorganism. Likewise, a “native promoter” or “endogenous promoter” is a promoter that is naturally found in a host microorganism. In contrast, “exogenous promoter” or “heterologous promoter” is a promoter introduced into a host microorganism via a genetic construct and which was obtained from a microorganism different from host microorganism.

As used herein, “coding sequence” or “coding region” refers to the portion[s] of a gene's DNA or RNA that codes for protein.

The term “microorganism” used herein refers to organisms recognized in the art as “microorganisms”. Microorganisms contemplated in the present disclosure include bacteria, filamentous fungi, and yeast. Additional examples of microorganism that can be used according to the present disclosure are well known to a person of ordinary skill in the art and such embodiments are within the purview of the present disclosure.

As used herein, “amino acid” refers to an organic compound containing amine and carboxyl functional groups along with a side chain, more specifically an alpha-amino acid of the general formula H₂NCHRCOOH which is a standard biochemical building block of proteins as described herein. Amino acids can be a natural (for example the 21 well known and described amino acids) or non-natural, and can be amino acids produced by or otherwise present in eukaryotes and prokaryotes.

As used herein, “amino acid sequence” refers to a sequence of amino acids connected by peptide bonds. Amino acid sequences as used herein can encode for functional proteins of bacteria as described herein.

As used herein, “immunocomplex” refers to a complex formed by the interaction and binding of a primary antibody with an associated antigen.

As used herein, “amplicon” refers to a piece of DNA or RNA that is the source and/or product of amplification or replication events. It can be formed artificially, using various methods including polymerase chain reactions (PCR) or ligase chain reactions (LCR), or naturally through gene duplication. In this context, amplification refers to the production of one or more copies of a genetic fragment or target sequence, specifically the amplicon. As it refers to the product of an amplification reaction, amplicon is used interchangeably with common laboratory terms, such as “FOR product.”

The phrase “specifically binds”, when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister proteins. A variety of immunoassay formats may be used to select antibodies specifically immunoreactive with a particular protein or in a particular form. For example, Solid phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically, a specific or selective binding reaction will be at least twice background signal or noise and more typically more than 10 to 100 times background.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, Ig|D and IgE, respectively. Typically, the antigen-binding region of an antibody or its functional equivalent will be most critical in specificity and affinity of binding. See Paul, Fundamental Immunology.

A “label”, “detectable label,” or “detectable moiety” is a composition detectable by spectroscopic, photochemical, biochemical, immunochemical, chemical, or other physical means. For example, useful labels include ‘P. fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically, a detectable label is attached to a molecule (e.g., antibody) with defined binding characteristics (e.g., a polypeptide with a known binding specificity), so as to allow the presence of the molecule (and therefore its binding target) to be readily detectable.

Two nucleic acid sequences or polypeptides are said to be “identical” if the sequence of nucleotides or amino acid residues, respectively, in the two sequences is the same when aligned for maximum correspondence as described below. The terms “identical’ or percent “identity.” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative Substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative Substitution is given a score of Zero, a conservative Substitution is given a score between Zero and 1. The scoring of conservative Substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical” used in the context of two nucleic acids or polypeptides, refers to a sequence that has at least 60% sequence identity with a reference sequence. Alternatively, percent identity can be any integer from 60% to 100%. Some embodiments include at least: 60%, 65%, 70%, 75%, 80%, 85%, 90%, 91%, 92%, 93%, 94% 95%, 96%, 97%, 98%, or 99%, compared to a reference sequence using the programs described herein; preferably BLAST using standard parameters.

As used herein, “gene product” refers to a immature or mature mRNA or peptide sequence that is transcribed or translated respectively ultimately from a nucleotide sequence that encodes a gene.

As used herein, the term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its sequence and/or conformation, and thereby can inhibit or suppress functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

Aptamers

Nucleic Acid Aptamers

Nucleic acid aptamers are typically oligonucleotides ranging from 15-50 bases in length that fold into defined secondary and tertiary structures, such as stem-loops or G-quartets. Nucleic acid aptamers preferably bind the target molecule with a K_{d} less than 10⁻⁶, 10⁻⁶, 10⁻¹⁰, or 10⁻¹². Nucleic acid aptamers can also bind the target molecule with a very high degree of specificity. It is preferred that the nucleic acid aptamers have a K_{d} with the target molecule at least 10, 100, 1000, 10,000, or 100,000 fold lower than the K_{d} of other non-targeted molecules.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. An aptamer database containing comprehensive sequence information on aptamers and unnatural ribozymes that have been generated by in vitro selection methods is available at aptamer.icmb.utexas.edu.

A nucleic acid aptamer generally has higher specificity and affinity to a target molecule than an antibody. Accordingly, a nucleic acid aptamer can specifically, directly, and firmly bind to a target molecule. Since the number of target amino acid residues necessary for binding may be smaller than that of an antibody, for example, a nucleic acid aptamer is superior to an antibody, when selective suppression of functions of a given protein among highly homologous proteins is intended.

Non-modified nucleic acid aptamers are cleared rapidly from the bloodstream, with a half-life of minutes to hours, mainly due to nuclease degradation and clearance from the body by the kidneys, a result of the aptamer's inherently low molecular weight. This rapid clearance can be an advantage in applications such as in vivo diagnostic imaging. However, several modifications, such as 2′-fluorine-substituted pyrimidines, polyethylene glycol (PEG) linkage, etc. are available to increase the serum half-life of aptamers to the day or even week time scale.

Another approach to increase the nuclease resistance of aptamers is to use a Spiegelmer. Spiegelmers are ribonucleic acid (RNA)-like molecules built from the unnatural L-ribonucleotides. Spiegelmers are therefore the stereochemical mirror images (enantiomers) of natural oligonucleotides. Like other aptamers, Spiegelmers are able to bind target molecules such as proteins. The affinity of Spiegelmers to their target molecules often lies in the pico-to nanomolar range and is thus comparable to antibodies. In contrast to other aptamers, Spiegelmers have high stability in blood serum since they are less susceptible to be cleaved hydrolytically by enzymes. Nonetheless, they are excreted by the kidneys in a short time due to their low molar mass. Unlike other aptamers, Spiegelmers may not be directly produced by the SELEX method. This is because L-nucleic acids are not amenable to enzymatic methods, such as polymerase chain reaction. Instead, the sequence of a natural aptamer identified by the SELEX method is determined and then used in the artificial synthesis of the mirror image of the natural aptamer.

Peptide Aptamers

Peptide aptamers are proteins that are designed to interfere with other protein interactions inside cells. They consist of a variable peptide loop attached at both ends to a scaffold. This double structural constraint greatly increases the binding affinity of the peptide aptamer to levels comparable to an antibody.

The variable loop length is typically composed of about ten to twenty amino acids, and the scaffold may be any protein which has good solubility. Currently, the bacterial protein Thioredoxin-A is the most used scaffold protein, the variable loop being inserted within the reducing active site, the two Cysteines lateral chains being able to form a disulfide bridge.

Peptide aptamer selection can be made using different systems, but the most used is currently the yeast two-hybrid system. Peptide aptamer can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### DISCUSSION

Described herein are methods and kits for detection of HLB and/or Ca. L. asiaticus infection in a subject. As described herein, methods and kits as described herein utilize detection of a combination of at least two or more secreted Ca. L. asiaticus effector proteins to ensure robust results. Methods and kits as described herein can detect a combination of at least two or more secreted Ca. L. asiaticus effector proteins of the SEQ. ID. NOs (or substantially identical variants thereof) according to the discussion and examples below.

According to the present disclosure, expression of Ca. L. asiaticus effector proteins in host subjects has been validated at various time points, including up to a year of controlled infection. Monitoring the expression of Ca. L. asiaticus effector proteins in host subjects by methods and kits as described herein can be utilized for the early detection of citrus greening disease, in some instances as little as 6 hours after infection, and in others, as much as 7 days after infection.

Although some secreted Ca. L. asiaticus effector protein expression is ubiquitous across citrus plants, other secreted Ca. L. asiaticus effector protein expression can be species-specific.

According to embodiments of the present disclosure, the presence of effector protein nucleotide sequences (such as mRNA) and/or the expression of two or more Ca. L. asiaticus effector proteins in host subjects can be detected as an indication of HLB. Methods and kits as described herein can detect expression, at 6 hours post infection, of Ca. L. asiaticus effector protein gene CLIBASIA_03695 in combination with one or more of CLIBASIA_00460, CLIBASIA_04580, CLIBASIA_05315, and CLIBASIA_05320. Nucleotide gene sequences as well as the full amino acid and mature amino acid sequences are provided for these genes in the examples below.

In other embodiments according to the present disclosure, methods and kits as described herein can detect the presence of nucleotide sequences (such as mRNA) or protein expression, at 7 days post infection, of two or more Ca. L. asiaticus effector protein genes selected from the group consisting of CLIBASIA_00420, CLIBASIA_00525, CLIBASIA_03875, CLIBASIA_04425, CLIBASIA_04900, and CLIBASIA_05640.

In an embodiment, methods and kits as described herein can detect the presence of nucleotide sequences or protein expression of a combination of two or more of CLIBASIA_03875, CLIBASIA_04900, and CLIBASIA_05640.

In other embodiments according to the present disclosure, methods and kits as described herein can detect the presence of nucleotide sequences (such as mRNA) or protein expression, at a first time point, of Ca. L. asiaticus effector protein gene CLIBASIA_03695 in combination with one or more of CLIBASIA_00460, CLIBASIA_04580, CLIBASIA_05315, and CLIBASIA_05320, and the presence of nucleotide sequences (such as mRNA) or protein expression, at a second time point, of two or more Ca. L. asiaticus effector protein genes selected from the group consisting of CLIBASIA_00420, CLIBASIA_00525, CLIBASIA_03875, CLIBASIA_04425, CLIBASIA_04900, and CLIBASIA_05640. In certain embodiments, the presence of nucleotide sequences (such as mRNA) or protein expression, at a second time point, are of two or more Ca. L. asiaticus effector protein genes selected from the group consisting of CLIBASIA_03875, CLIBASIA_04900, and CLIBASIA_05640. The second time point can be later than the first time point.

In other embodiments according to the present disclosure, methods and kits as described herein can detect the presence of one or more or two or more amino acid sequences of expressed effector proteins encoded by the genes CLIBASIA_00460, CLIBASIA_03695, CLIBASIA_04025, CLIBASIA_04580, CLIBASIA_05315, CLIBASIA_05320, CLIBASIA_00420, CLIBASIA_00525, CLIBASIA_03875, CLIBASIA_04330, CLIBASIA_04425, CLIBASIA_04900, or CLIBASIA_05640. At minimum, methods and kits as described herein detect the presence of amino acid sequences encoded by the gene CLIBASIA_03695 (SEQ ID NO:32 or 33 below, or substantially identical variants thereof).

Embodiments of kits are further disclosed below, but embodiments of combinations of gene products detectable by kits as described herein are as follows for the following genes and/or proteins:

CLIBASIA_03695 and CLIBASIA_00460;

CLIBASIA_03695 and CLIBASIA_04025;

CLIBASIA_03695 and CLIBASIA_04580

CLIBASIA_03695 and CLIBASIA_05315;

CLIBASIA_03695 and CLIBASIA_05320;

CLIBASIA_03695 and CLIBASIA 00420;

CLIBASIA_03695 and CLIBASIA_00525;

CLIBASIA_03695 and CLIBASIA_03875;

CLIBASIA_03695 and CLIBASIA_04330;

CLIBASIA_03695 and CLIBASIA_04425;

CLIBASIA_03695 and CLIBASIA_04900; and

or CLIBASIA_03695 and CLIBASIA_05640.

In certain aspects, kits can consist of a combination of CLIBASIA_03695 in addition with those above. In certain aspects, kits can comprise a combination of CLIBASIA_03695 in addition with those above. In additional embodiments, methods and kits as described herein detect products of CLIBASIA_03695 and any one or more of CLIBASIA_00460, CLIBASIA_04025, CLIBASIA_04580, CLIBASIA_05315, CLIBASIA_05320, CLIBASIA_00420, CLIBASIA_00525, CLIBASIA_03875, CLIBASIA_04330, CLIBASIA_04425, CLIBASIA_04900, or CLIBASIA_05640

Methods and kits as described herein can detect biological targets from a sample or from an isolated sample. A sample can be from a plant or an insect for example a citrus plant or a psyllid. A sample or isolated sample can be comprised of a portion of a plant (for example a leaf, root, or part thereof) or insect (for example a wing, legs, antennae, or part thereof). A portion is anything less than the whole.

In embodiments according to the present disclosure, citrus plants as described herein can be one or more of Citrus sinensis, Citrus reticulata, Citrus excels. In embodiments according to the present disclosure, a citrus plant is a Duncan grapefruit, Washington navel orange, citron mandarin, or Cleopatra mandarin.

Psyllids as described herein can be Diaphorina citri Kuwayama. In an embodiment, a psyllid is an Asian citrus psyllid (ACP).

In embodiments according to the present disclosure, biological targets are comprised of nucleic acid sequences and/or amino acid sequences. Without intending to be limiting, nucleic acid sequences can be messenger RNA sequences transcribed from the genome of the Ca. L. asiaticus bacterium following host (plant or insect) infection. Amino acid sequences can be full or mature sequences of proteins translated from nucleic acid sequences as described herein.

In certain aspects, biological targets can be isolated from samples of subjects infected with or otherwise carrying Ca. L. asiaticus bacterium. In certain aspects, biological targets can be isolated from samples of subjects at risk for infection by or otherwise carrying Ca. L. asiaticus bacterium.

Isolation of biological targets can be accomplished by well-established methods in the art as understood by the skilled artisan. Isolation of nucleic acid sequences can be done according to embodiments presented in the examples below. For example, without intending to be limiting, DNA samples can be isolated utilizing commercially available kits, such as the DNeasy Plant Mini Kit available from QIAGEN. RNA samples can be extracted using TRIzol® and associated protocols. RNA purification from extracted samples can be accomplished using commercially available kits such as the RNeasy Plant Mini Kit from QIAGEN. cDNA can be generated from isolated and purified RNA samples using commercially available kits such as the High-Capacity cDNA Reverse Transcription Kit from Applied Biosystems®. Examples of primers that can be utilized for cDNA and amplicon generation can be seen in Table 2 below (SEQ ID Nos:85-150). Kits as described herein, in embodiments, can comprise primers directed at forming and detecting amplicons of the following combinations:

CLIBASIA_03695 and CLIBASIA_00460;

CLIBASIA_03695 and CLIBASIA_04025;

CLIBASIA_03695 and CLIBASIA_04580

CLIBASIA_03695 and CLIBASIA_05315;

CLIBASIA_03695 and CLIBASIA_05320;

CLIBASIA_03695 and CLIBASIA_00420;

CLIBASIA_03695 and CLIBASIA_00525;

CLIBASIA_03695 and CLIBASIA_03875;

CLIBASIA_03695 and CLIBASIA_04330;

CLIBASIA_03695 and CLIBASIA_04425;

CLIBASIA_03695 and CLIBASIA_04900; and

or CLIBASIA_03695 and CLIBASIA_05640.

Kits can also comprise primers for amplification and/or detection of a control gene, for example 16s.

Detection of isolated biological targets by methods and kits as described herein can be accomplished by well-established methods in the art, and the detection of mRNA, cDNA, DNA, and amino acid sequences is well known by a variety of methods. For example, nucleic acid sequences can be detected through the generation of amplicons with sequence-of-interest specific primers using polymerase chain reaction.

Primers for amplicon generation (cDNA and/or genomic DNA) can be generated against a nucleic acid sequence of interest (for example the full-length nucleotide sequences below, or nucleotide sequences encoding amino sequences as disclosed below) by one of skill in the art using any number of bioinformatic tools readily available, for example and without intending to be limiting, the Primer-BLAST tool at the U.S. National Library of Medicine National Center for Biotechnology Information website.

Amplicons can be generated using a variety of methods according to those established in the art. Without intending to be limiting, for example, amplicons can be generated using standard polymerase chain reaction (PCR) and common thermocyclers, or by other methods, such as quantitative PCR (qPCR) or quantitative real-time PCR (qRT-PCR, also referred to as real-time PCR RT-PCR) using SYBR® or Taqman® chemistries.

Amplicons can be detected by well-established methods in the art as understood by the skilled artisan. Such methods can include standard electrophoresis using ethidium-bromide agarose gels, detection using detectably-labeled primers or amplicons incorporating SYBR® chemistry, Taqman® chemistry, or other labels. In certain aspects, primers as described herein can be conjugated to a detectable label.

Examples of primer sequences that can be part of kits as described herein and that can be used to generate amplicons as described herein can be found in Table 2 below (SEQ ID NOs:85-150). One of skill in the art would recognize that primers can consist of SEQ ID NOs:85-150, can comprise SEQ ID NOs:85-150, be substantially similar to such. Other primers can be generated utilizing well-known techniques in the art as described herein.

Peptide sequences (or polypeptides), as used herein, refers to sequences of amino acids, wherein the amino acids of the sequence linked together to one another by peptide bonds. Peptide sequences can encode for an immature protein, a mature protein, or any portion thereof.

Peptide sequences can be isolated from subjects as described herein by methods according to those known in the art. Isolated peptide sequences can comprise full-length or mature amino acid sequences according to the SEQ ID NOs. disclosed in the examples below, or sequences substantially identical to such.

Isolated peptide sequences can be detected according to methods as known in the art, and to methods and kits as described herein. Primary antibodies that recognize amino acid sequences according to the SEQ ID NOs. disclosed in the examples below, or sequences substantially identical to such, can be incubated with isolated peptide sequences to form immunoconjugates, which can then be incubated with secondary antibodies that are detectably labeled by standard detection methods (for example enzymatically, such as by horseradish peroxidase, or fluorescent microscopy or other detection methods thanks to a detectable label conjugated to the antibody).

Antibodies as described herein can be monoclonal or polyclonal and raised in a host such as rabbit, mouse, goat, rat, horse, human, and those commonly known in the art. In embodiments, antibodies are rabbit polyclonal antibodies conjugated to HRP or otherwise conjugated to HRP-detectable labels. Primary antibodies as described herein can be made by methods known in the art, for example by immunizing an animal (such as rabbit, hamster, guinea pigs, chicken, sheep, pig, donkey, horse, mouse, rat) with an adjuvant and isolate protein of a sequence as described herein, bleeding the animal, and isolating the polyclonal antibodies from the blood that specifically recognize a sequence of the protein injected.

In certain aspects, primary polyclonal antibodies conjugated to HRP are utilized without secondary antibodies.

Such antibodies as described herein can be components of kits as described herein.

Antibody reagents can be used in assays to detect the presence of, or protein expression levels, for the at least one secreted protein in a citrus sample using any of a number of immunoassays known to those skilled in the art. Immunoassay techniques and protocols are generally described in Price and Newman, “Principles and Practice of Immunoas say, 2nd Edition, Grove's Dictionaries, 1997; and Gosling, “Immunoassays: A Practical Approach, Oxford University Press, 2000. A variety of immunoassay techniques, including competitive and non-competitive immunoassays, can be used. See, e.g., Self et al., Curr. Opin. Biotechnol. 7:60-65 (1996). The term immunoassay encompasses techniques including, without limitation, enzyme immunoassays (EIA) Such as enzyme multiplied immunoassay technique (EMIT), enzyme-linked immunosorbent assay (ELISA), IgM antibody capture ELISA (MAC ELISA), and microparticle enzyme immunoassay (MEIA), capillary electrophoresis immunoassays (CEIA); radioimmunoassays (RIA); immunoradiometric assays (IRMA), fluorescence polarization immunoassays (FPIA); and chemiluminescence assays (CL). If desired. Such immunoassays can be automated. Immunoassays can also be used in conjunction with laser induced fluorescence. See, e.g., Schmalzing et al., Electrophoresis, 18:2184-93 (1997); Bao, J. Chromatogr: B. Biomed. Sci., 699:463-80 (1997). Liposome immunoassays, such as flow injection liposome immunoassays and liposome immunosensors, are also Suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the protein concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

In some embodiments, the immunoassay includes a membrane-based immunoassay Such as a dot blot or slot blot, wherein the biomolecules (e.g., proteins) in the sample are not first separated by electrophoresis. In such an assay, the sample to be detected is directly applied to a membrane (e.g., PVDF membrane, nylon membrane, nitrocellulose membrane, etc). Detailed descriptions of membrane-based immunoblotting are found in, e.g., Gallagher, S R. “Unit 8.3 Protein Blotting:Immunoblotting. Current Protocols Essential Laboratory Techniques, 4:8.3.1-8.3.36 (2010).

Specific immunological binding of the antibody to the protein of interest can be detected directly or indirectly. Direct labels include fluorescent or luminescent tags, metals, dyes, radionuclides, and the like, attached to the antibody. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), B-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic Substrate p-nitrophenyl phosphate, for example, which yields a soluble product readily detectable at 405 nm. Similarly, a f-galactosidase detection system can be used with the chromogenic Substrate o-nitro phenyl-B-D-galactopyranoside (ONPG), which yields a soluble product detectable at 410 nm. An urease detection system can be used with a Substrate Such as urea-bromocresol purple (Sigma Immunochemicals; St. Louis, Mo.).

### Kits

Also disclosed herein are kits comprising any one of the compositions above in a suitable amount or assay configuration to detect one or more of or two or more of SEQ ID NOs:5, 6, 32, 33, 38, 39, 59, 60, 77, 78, 80, 81, 2, 3, 11, 12, 35, 36, 50, 51, 53, 54, 83, 84, or substantially identical variants thereof. At minimum, a kit as described herein is configured as an assay to detect or has at least one antibody that recognizes SEQ ID NO:32, 33, or both. In one embodiment, the kit further comprises instructions for use. In other embodiments, the composition is lyophilized such that addition of a hydrating agent (e.g., buffered saline) reconstitutes the composition for use. In an embodiment, the kit is an ELISA, or otherwise configured as an ELISA assay. In embodiment, the kit is configured as a microtiter plate-based assay. The kit can be configured as an immunoassay containing at least one detectably-labeled component (for detection visually, fluorescently, by radio-isotope, and such), such as an antibody.

Kits as described herein are configured for detection of Candidatus Liberibacter bacteria in either citrus or psyllid samples, or both in certain aspects. In an embodiment, a kit is configured as a laboratory microtiter plate-based assay that serves as a more sensitive and quantitative assay than other modalities, and in certain aspects, could be made available to growers as part of a detection service where growers submit samples to a lab for analysis.

In another embodiment, the kit can be a field deployable hand-held device that could be used by growers for rapid on-site in-field detection of CLas effector proteins in plant or insect samples. Each kit can detect the presence of C. Liberibacter species proteins in asymptomatic tissues allowing an early detection strategy. ELISA is a well-established method invented in 1971 and its durability advantages and limitations are well documented as it is used throughout the world in numerous diagnostic assays. The ability to detect C. Liberibacter infections in asymptomatic citrus and psyllid samples can allow growers to make decisions about tree removal and other prophylactic practices that can reduce/block the spread of HLB and keep the trees healthier longer.

This kit could be configured for the analyses of leaves, roots, and/or fruit samples, and can contain materials and reagents necessary to carry out the following method, for example scissors, a razor, ceramic beads, tubes, and other such. For leaf sample preparation, 100 mg of petiol and midrib were cut into small piece and transferred into 8-strip tubes having 3 ceramic beads per each well. Samples in the Tubes were chilled in liquid nitrogen and ground using Genogrinder at 1600 rpm for 1 min. Solution was spin down for 5 min at 4000 rpm and 500 ul of water was added, mixed using Genogrinder at 1200 rpm for 30 sec before spinning down for 10 min at 4000 rpm again. Supernatant were transferred and can be used as the sample solution for ELISA kits and dip stick kit. For root sample, lateral root of the plant was collected and prepared same as leaf sample. For fruit sample, juice was squeezed into a 50 ml conical tubes. The proposed on-site in-field detection test kit will be based on lateral flow immunochromatographic assay. The signal intensity of test line is proportional to the concentration of effector protein in samples. Briefly, extract sample by putting the leaf or root samples or drop of orange juice into provided sample extraction bags containing extraction buffer and macerating them. Place the test strip into the extract for a predetermined time (typically 5-10 minutes) and interpret results by comparing intensity on the reference card provided.

In an embodiment, a kit as described herein can comprise two or more reverse transcriptase primers wherein each of the reverse transcriptase primers is uniquely complementary to a nucleotide sequence having a SEQ ID NO (or substantially identical variant) as disclosed in the examples below. The kit can further comprise two or more pairs of polymerase chain reaction primers, wherein each of the pairs are configured to uniquely amplify a complementary nucleotide sequence, the complementary nucleotide sequence complementary to a nucleotide sequence having a SEQ ID NO (or substantially identical variant) as disclosed in the examples below. Such primers can be present in the kit in lyophilized form or other such form as known in the art.

In certain embodiments, the kit can be an immunoassay configured for rapid visual confirmation of the presence of one or more effector proteins having an amino acid sequence of a SEQ ID NO (or substantially identical variant) as disclosed in the examples below. In certain embodiments, the kit can be an immunoassay configured for rapid visual confirmation of the presence of two or more effector proteins having an amino acid sequence of a SEQ ID NO (or substantially identical variant) as disclosed in the examples below. In such embodiments, the kit can be configured as a spot-ELISA assay. Kits such as these can be used with samples from subjects such as sap, leaf, and root samples, but in particular sap as sap would require minimal sample processing. Antibodies for the detection of the effector proteins can be present on an immobilize support, such as a test strip.

The primary and/or antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

### Methods of Use

A further aspect of the present disclosure encompasses methods of using a kit disclosed herein or method of detection of HLB in a subject. Methods as described herein can comprise administering a sample from a subject in need thereof to a kit as described herein. The kit of methods as disclosed herein can be configured as an immunoassay sensitive to one or more amino acid sequences as disclosed herein. The kit of methods as disclosed herein can be configured as an ELISA or spot-ELISA assay. The sample can be a sap, root, or leaf sample. The subject in need thereof can be a citrus plant or psyllid that is infected by or at risk for infection by Ca. L. asiaticus.

In certain aspects, a method of detecting citrus huanglongbing (HLB) in a subject at risk for contracting HLB, can comprise: providing a sample from a subject; isolating a plurality of biological targets from the sample; isolating effector targets from the plurality of biological targets, if present in the isolated sample; and detecting the isolated effector targets. The subject can be a citrus or psyllid. The subject can be a citrus or psyllid infected by Ca. L. asiaticus. The subject can be a citrus or psyllid at risk for infection by Ca. L. asiaticus. The sample can comprise a portion of a leaf, root, or sap of the subject. The isolated biological targets comprise nucleic acid sequences or amino acid sequences. Isolating the effector targets can comprise providing two or more reverse transcriptase primers configured to amplify two or more of SEQ ID NOs. as described below or substantially identical variants thereof, wherein each of the reverse transcriptase primers recognizes an effector target unique to the primer; combining the reverse transcriptase primers with the isolated biological targets to create complementary DNA (cDNA) through reverse transcription of mRNA of effector targets corresponding to SEQ ID NO.s as described below or substantially identical variants thereof; and amplifying the cDNA. Detecting the isolated effector targets can comprise providing two or more pairs of polymerase chain reaction (PCR) primers configured to amplify SEQ ID NO.s as disclosed below or substantially identical variants thereof, wherein each of the primer pairs recognizes an effector target unique to the primer pair; combining the PCR primers with the isolated effector targets; amplifying the isolated effector targets target in the combined mixture through PCR. Isolating the effector targets can comprise providing two or more primary antibodies configured to recognize of two or more of SEQ ID NO.s as disclosed below or substantially identical variants thereof, wherein each of the primary antibodies recognizes an effector target unique to the antibody; and incubating the primary antibodies with the isolated biological targets to form two or more immunocomplexes. Detecting the isolated effector targets can comprise providing two or more secondary antibodies, wherein each of the secondary antibodies recognizes an immunocomplex unique to the antibody; and incubating the secondary antibodies with the immunocomplex.

Further described herein are embodiments of methods for detection of citrus huanglongbing (HLB) in a subject in need thereof. Methods as described herein comprise providing a sample; extracting proteins from the sample; administering two or more antibodies to the extracted proteins, wherein the two or more antibodies are configured to recognize different amino acid sequences encoding effector proteins from Candidatus Liberibacter asiaticus selected from the group consisting of: SEQ ID NOs:5, 6, 32, 33, 38, 39, 59, 60, 77, 78, 80, 81, 2, 3, 11, 12, 35, 36, 50, 51, 53, 54, 83, 84, or substantially identical variants thereof; forming a plurality of immunocomplexes by incubating the mixture of extracted proteins and two or more antibodies; detecting the immunocomplexes; determining from the detected immunocomplexes a disease status; and outputting the disease status.

One of the antibodies of methods as described herein recognizes SEQ ID NO:32, 33, or substantially identical variants thereof. Antibodies as described herein are detectably labeled.

The subject in need thereof is an infected citrus plant or psyllid insect or citrus plant or psyllid insect at risk for infection.

Described herein are methods for detection of citrus huanglongbing (HLB) in a subject in need thereof, comprising: providing a sample; extracting proteins from the sample; administering one or more antibodies to the extracted proteins, wherein the one or more antibodies are configured to recognize at least an amino acid sequence encoding an effector protein from Candidatus Liberibacter asiaticus of SEQ ID No: 32, 33, or substantially identical variants thereof; forming a plurality of immunocomplexes by incubating the mixture of extracted proteins and one or more antibodies; detecting the immunocomplexes; determining from the detected immunocomplexes a disease status; and outputting the disease status. The method can further comprise administering one or more antibodies configured to recognize SEQ ID Nos.:5, 6, 38, 39, 59, 60, 77, 78, 80, 81, 2, 3, 11, 12, 35, 36, 50, 51, 53, 54, 83, 84, or substantially identical variants thereof.

The sample can be a leaf, root, or sap sample. The one or more antibodies are detectably labeled. The subject in need thereof is an infected citrus plant or psyllid insect or citrus plant or psyllid insect at risk for infection.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Background

Huanglongbing (HLB) is currently the most devastating disease among citrus crops, significantly impacting the production in the United States and the industry worldwide. In the U.S. it is associated with the bacterium Candidatus Liberibacter asiaticus (Ca. L. asiaticus). Ca. L. asiaticus is an α-proteobacterium restricted in citrus to the phloem cells and is transmitted by the insect vector Asian citrus psyllid (ACP, Diaphorina citri). Likely associated with its intracellular nature, Ca. L. asiaticus is fastidious and it has not been maintained in sustainable culture in axenic conditions [1, 2]. Therefore, Koch's Postulate remains incomplete for this pathogen and studies related to bacterial pathogenesis are especially challenging. However, complete genome sequences of several Ca. L. asiaticus isolates have been reported and made publicly available [3-6]. Sequence data has provided useful genetic information on the evolution of the bacterium and permitted inferences on virulence mechanisms and essential metabolism. The genomic dataset includes many putative genes that can be subject to reverse genetics for functional analysis.

Plants have evolved layered innate immune systems that function via inter- and intracellular defenses [7]. Cell surface receptors can perceive pathogen-derived molecules such as pathogen-associated molecular patterns (PAMPs) which then activate 65 PAMP-triggered immunity (PTI). In turn, pathogens secrete effector proteins that suppress PTI or regulate plant physiology to facilitate disease development, which results in effector-triggered susceptibility (ETS). Incompatible hosts maintain resistance (R) genes capable of recognizing the effectors and then causing rapid immune responses categorized as effector-triggered immunity (ETI). In the Ca. L. asiaticus genome, a set of flagellum-associated genes have been identified [3] including the Fla gene which includes the 22-amino acid PAMP flg22 [8]. Assays with citrus hosts indicated Ca. L. asiaticus flg22 elicited plant defenses that differed in strength between susceptible and tolerant citrus suggesting this may have a role in disease tolerance [9]. In the study of Ca. L. asiaticus effectors, in silico genome searches uncovered a repertoire of candidates [10, 11]. Protein function studies revealed that the effector CLIBASIA_05315 induced ETI-like reactions in Nicotiana benthamiana [11] and contributed to excessive cellular starch accumulation, a typical physiological disorder associated with HLB [12]. Recently, a papain-like cysteine protease was identified to be the target of CLIBASIA_05315 in citrus and this uncovered an interesting aspect of virulence mechanism for HLB [13].

Effector biology is emerging as an important aspect of the investigation on plant-pathogen interactions, as secreted effector proteins play many roles in the pathogenicity that lead to initial infection and host colonization. An understanding of fundamental effector biology is key to revealing pathogen evolution to achieve virulence, including subcellular localizations, mechanisms of host cell modulation, and in planta binding targets including susceptible and R genes [14-16]. Importantly, characterization of effectors provides guidance on and accelerates crop resistance breeding. In combating potato late blight caused by Phytophthora infestans, increasing numbers of R gene-effector pa 88 irs have been identified [17, 18] and implemented in breeding to select for naturally-occurring R genes that provide durable resistance [19]. Alternatively, R genes identified from biochemical or genetic approaches can be cloned and used in transgenic production of resistant cultivars, a strategy that has been demonstrated in tomato [20], rice [21], potato [22] and alfalfa [23] as examples.

Genetic resistance to HLB appears to be lacking within the Citrus genus. However, disease tolerance as shown by mild symptom and less impairment on tree development have been observed in both controlled inoculations [24, 25] and field pathogen exposure [26-28]. In Florida, an evaluation of 83 Citrus and Citrus relatives accessions with 6-years of field exposure of HLB identified citron (Citrus medica) as a genetic source of tolerance [28]. The Citrus relatives trifoliate orange (Poncirus trifoliata) and its hybrids have been an important resource as rootstocks and showed marked HLB tolerance/resistance in multiple studies [27, 29, 30]. On the other hand, commercially important citrus types such as grapefruits (C. paradisi), sweet oranges (C. sinensis) and mandarins (C. reticulata) are all negatively impacted by HLB, but disease severity varies greatly. Several studies report potentially useful tolerance in some mandarins [24, 26, 31], which also appears to be heritable (Stover, unpublished results).

In silico analysis of the Ca. L. asiaticus genome was performed which identified a total of 28 effector candidates. The mRNA of 20 effectors were detected in Ca. L. asiaticus-infected citrus by RT-qPCR. Using a detached leaf assay for insect-mediated bacterial transmission, effector mRNA could be detected from 6 hours to 7 days after ACP infestation, and the number of effectors detected was positively 110 correlated with the bacterial titer. Subsequently, the expression of effectors was compared in six citrus types with different HLB tolerance levels, including citron, Duncan grapefruit, Cleopatra mandarin, Pomeroy trifoliate, Washington navel orange and Carrizo citrange. Results indicated some effector candidates had relatively high expression in multiple citrus genotypes regardless of tolerance levels, while other effectors had host-specific expression patterns. The effectors expression was also compared between leaf and root tissues in own-rooted citrus which had been infected with Ca. L. asiaticus for more than a year. Several candidate effectors were found to have relatively high transcriptional level in leaf tissue and some had higher expression in roots. Taken together, these studies provide guiding information on Ca. L. asiaticus utilization/deployment of effectors at early- and late-colonization stages and different tissues and hosts, which may lead to the selection of promising candidates for functional analysis and direct citrus resistance breeding.

### Methods

### Plants Materials

All of the plant materials were clonally produced and maintained at the US Horticultural Research Lab greenhouse facilities. Healthy seedlings of Duncan grapefruit, Cleopatra mandarin, citron, Pomeroy trifoliate, Washington naval orange and Carrizo citrange were grown in pots in the greenhouse using typical water, fertilizer and pesticide applications as for citrus nursery production, except that no insecticides were applied. For detached leaf inoculation, leaves for each citrus type were collected at similar maturity and size and used immediately for described experiments. The leaf and root analysis used citron, Duncan and Cleopatra plants derived from seed and inoculated more than a year earlier, verified as Ca. L. asiaticus-infected, and maintained in the quarantine greenhouse.

### Detached Leaf Inoculation and Sample Collection

The leaf samples were gently rinsed and dried in the lab, with the petioles placed in water in 1.5 mL centrifuge tubes sealed with Parafilm. Each detached leaf, with its petiole in water, was placed in a 50 mL plastic centrifuge tube with a mesh top and was exposed to 10 psyllids collected from colonies maintained on Ca. L. asiaticus infected plants. Tubes containing leaves and psyllids were kept on the lab bench with supplemental light of 16 h per day. At the time of destructive sampling, the ACP were removed by vacuum and the leaves were wiped clean before sampling. A group of 10-15 leaves were collected each at 6 h, 1 d, 3 d and 7 d post ACP infestation, or for each citrus genotype at the 7 d time point. The midrib tissues were excised for PCR quantification of Ca. L. asiaticus 16s rDNA, and the remaining leaf tissues were placed initially in liquid nitrogen and then stored at −80° C. for RNA isolation and expression analysis.

### Whole Plant Inoculation and Sample Collection

Ca. L. asiaticus-infected citron, Duncan and Cleopatra plants, all own-rooted from seed, were inoculated by ACP infestation during a previous study in 2016 and maintained in a greenhouse along with uninfected controls [9]. Briefly, six citrus plants with young flushes were infested by 400 Ca. L. asiaticus positive ACP or negative ACP (as mock inoculation) in dome cages for 2 weeks. Then insects were removed by aspiration, and plants were treated with carbaryl insecticide and greenhouse growth condition were resumed. A pool of 4 to 6 leaves were collected from each plant and used for leaf analysis of effector expression. Fibrous roots were sampled peripherally from the root mass of each plant, rinsed and stored at −80° C. for analysis.

Determination of Ca. L. Asiaticus Bacterial Titer

To quantify Ca. L. asiaticus in citrus leaves, DNA was isolated from midrib of leaves or fibrous roots using DNeasy plant mini kit (Qiagen, Gaithersburg, Md., USA), and the quantity and quality were determined by a NanoDrop Spectrophotometer (Thermo Scientific, Wilmington, Del., USA). For each sample, 100 μg of DNA was used for qPCR of Ca. L. asiaticus 16s rDNA using Las Long primers and SYBR chemistry (Thermo Fisher Scientific, Waltham, Mass., USA) and an AB17500 thermal cycler (Applied Biosystems, Foster City, Calif., USA). The threshold cycle (Ct) values were used to calculate bacterial titer using the standard curve method [9].

### Expression Analysis of Effector Candidates by RT-qPCR

The total RNA was isolated from Ca. L. asiaticus positive leaf or root samples using TriZol reagent (Invitrogen, Carlsbad, Calif., USA) following the manufacturer's instructions. On-column DNase treatment and RNA purification were performed using RNeasy Plant Mini Kit (Qiagen). The quantity and quality of RNA were determined using a NanoDrop Spectrophotometer (Thermo Scientific). Subsequently, 1 μg of RNA was used for cDNA synthesis by QuantiTect Reverse Transcription Kit (Qiagen). A mix of SSPs (see Table 2 below) targeting Ca. L. asiaticus effectors, 16s and a citrus endogenous gene UPL7 was used for cDNA synthesis at a final concentration of 0.5 μM each. A 20 μL of reverse transcription reaction for each sample was performed according to the kit protocol. Subsequently, the cDNA was diluted to 5 ng/μL and 2 μL was used in each qPCR reaction together with forward and reverse SSPs (see Table 2 below) to amplify effector candidates.

### Results

Ca. L. asiaticus Secretome Analysis for Effector Candidate Identification

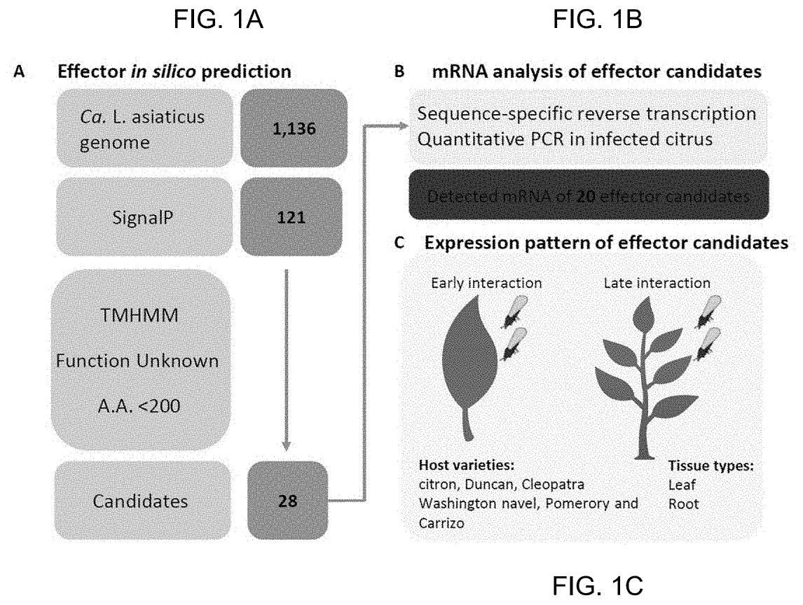

The Ca. L. asiaticus psy62 v1 genome (Genbank accession NC_012985.3) [3] was used for bioinformatic prediction of effectors. Based on common features of bacterial secreted proteins, the 1,136 protein-coding sequences were filtered using SignalP [32] to predict the presence of signal peptides (SP) which were then subjected to TMHMM v2 [33, 34] to eliminate proteins with transmembrane domains [35]. Subsequently, the candidates with fewer than 200 amino acids were selected, and homology was searched using the National Center for Biotechnology Information (NCBI) BLAST tool. The proteins without any annotated homologs were analyzed in this study. A group of 28 putative Ca. L. asiaticus effectors was identified and used for the following experiments (see Tables 1A-1B and FIGS. 1A-1C).

Detection of Putative Effectors by RT-qPCR in the Multiple Citrus Genotypes Infected by Ca. L. asiaticus

Sequence specific primers (SSPs) were designed for both reverse transcription and qPCR for greater specificity and higher capacity in amplification of effectors at low enrichment. Primers for reverse transcription were chosen at the 3′ end of the mRNAs to direct the synthesis of the first strand of cDNA. Forward and reverse primers were designed downstream of the cDNA synthesis primer binding region 197 (see Example 5 below). As one of the objectives was to determine if the expression pattern of effectors differed between HLB-susceptible, -tolerant and -resistant citrus, the SSPs efficacy were tested using multiple citrus species where specificity was defined as positive amplification in the Ca. L. asiaticus-infected tissue but not when uninfected. Hence RNA was isolated from Ca. L. asiaticus positive and negative leaves of citron, Duncan grapefruit and Cleopatra mandarin, Washington navel, Pomeroy trifoliate and Carrizo citrange for cDNA synthesis and qPCR. The SSPs of 20 effector candidates showed Ca. L. asiaticus specific amplification of targets in all citrus genotypes (see FIG. 1A and Table 2), whereas the remaining amplified in at least one of the uninfected citrus genotypes.

Effector Expression During Early Ca. L. asiaticus-Citrus Interactions and Bacterial Titer Dependent Effector mRNA Detection

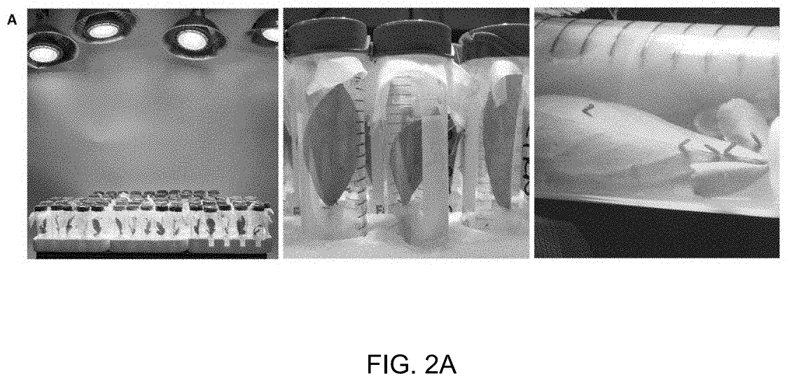

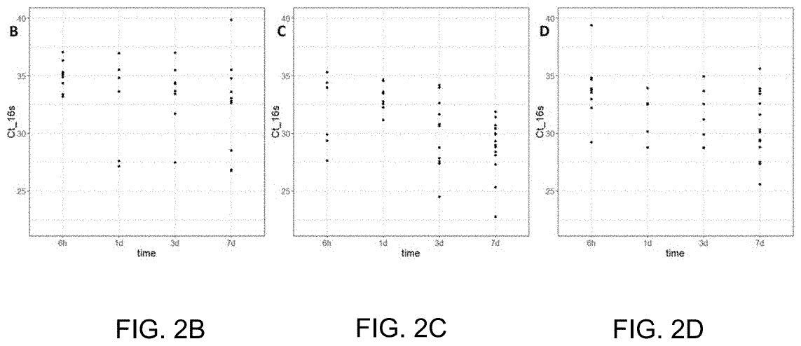

Due to the fastidious nature of this organism, common bacterial inoculation methods cannot be used to study the early interactions between Ca. L. asiaticus and citrus. This study used ACP infestation with detached leaves as previously described for inoculation [36] to study an early infection time course. To compare Ca. L. asiaticus effector expression in citrus genotypes with different HLB tolerance levels, citron, Duncan, and Cleopatra citrus were used. Leaves from each genotype were exposed to ACP feeding for 6 hours (h), 1, 3 and 7 days (d). Midrib DNA analysis indicated some successful bacterial transmissions at all of the time points and citrus genotypes, which provided multiple Ca. L. asiaticus positive samples for effector expression study (FIGS. 2A-2D). Three randomly selected infected samples at each time point and genotype were used for RT-qPCR, and showed detection of the mRNA of effectors at all the time points and genotypes studied (see Tables 3A-3C). The number of effectors detected by RT-qPCR was generally low in most of samples which made it difficult to apply mean calculations and statistics; although some effector showed expression across more than two time points such as ‘4025’ in citron and ‘5315’ in Duncan and Cleopatra (From here on the last 4 digits of Ca. L. asiaticus gene locus name are used for simplicity). Noticeably, two samples with higher bacterial titers at 6 h and 3 d in citron provided detectable mRNA of larger number of effectors (see Tables 3A-3C), which suggested that bacterial titer and number of effectors detected by RT-qPCR are positively correlated.

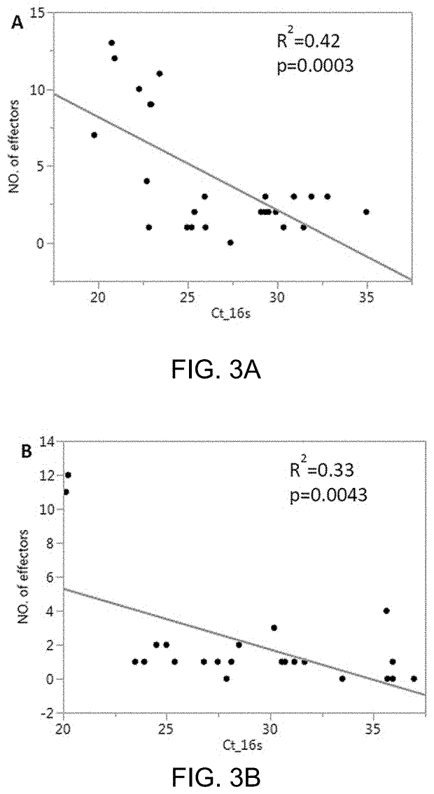

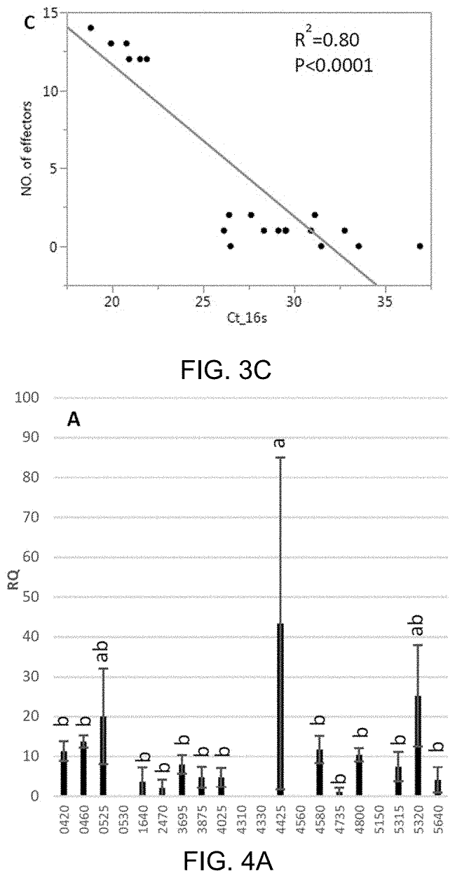

To test this aspect, additional Ca. L. asiaticus-infected samples at a later infection stage were included for a larger dataset and more diverse sample pools. These RNA samples were generated from a previous study on citrus transcriptome response to Ca. L. asiaticus at 2, 4, 6, 8 and 10 weeks (wk) after inoculation [9], from which multiple samples from each time point were used for effector mRNA detections. From the RT-qPCR data, the number of effectors expressed was counted and plotted against the Ct values quantifying Ca. L. asiaticus 16s in each sample and each genotype. The results indicated a significant linear relationship between the two groups of variables in the three citrus genotypes tested, where the liner model fit was highest in Cleopatra (R2=0.80) and lowest in Duncan (R2=0.33) (FIGS. 3A-3C).

Differential Effector Expression in Citrus Species with Various HLB Tolerance

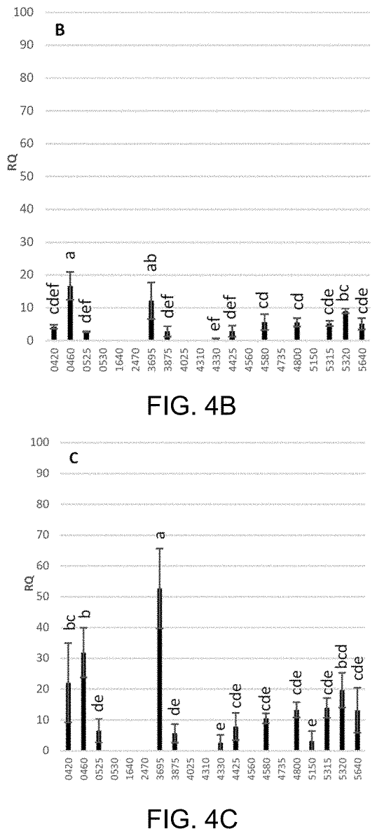

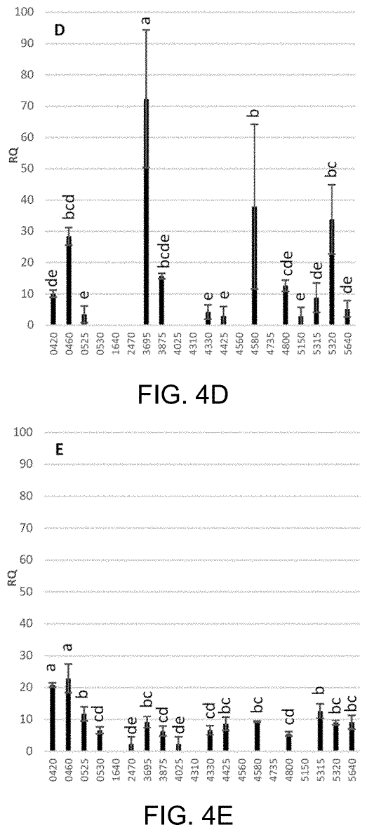

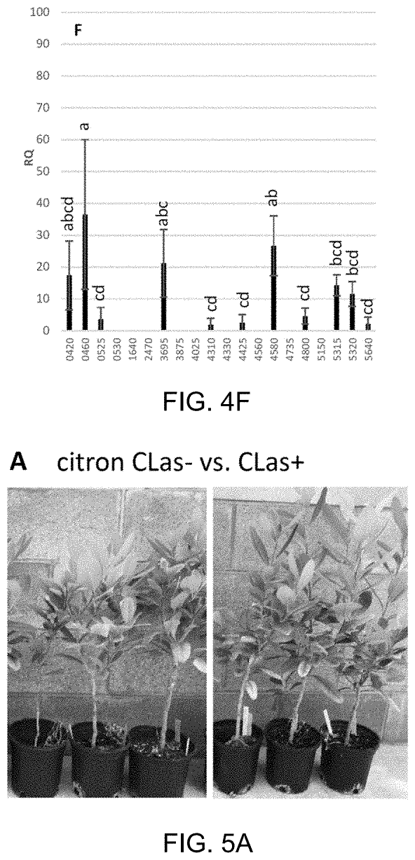

To determine if effector expression patterns are different between HLB tolerant, susceptible, and resistant hosts at the same infection stage, the detached leaf study used in two highly susceptible citrus genotypes (Duncan and Washington navel orange), two tolerant types (Cleopatra and citron), and two resistant genotypes (Pomeroy trifoliate and Carrizo citrange). Leaves of the six citrus genotypes were exposed to a 7-day infestation by the same ACP population for bacterial inoculation and only leaves which were Ca. L. asiaticus positive were used for effector analysis. The relative quantification (RQ) of effectors was compared within each citrus type to identify the higher expressed candidates that were likely to be critical for virulence. In the susceptible Duncan, only ‘4425’ had high expression while the level of other detected effectors were similar (FIG. 4A). Effectors ‘0460’ and ‘3695’ were the most expressed effectors in Washington Navel (FIG. 4B). In citron ‘3695’ showed the highest expression among all the effectors, and ‘0460’ and ‘0420’ were also highly ranked (FIG. 4C). Similarly, ‘3695’ was the highest expressed effector in Cleopatra, and ‘4580’ and ‘5320’ had higher expression than most of the others (FIG. 4D). The trifoliate orange Pomeroy showed ‘0420’ and ‘0460’ as the highest expressing group, followed by ‘0525’ and ‘5315’ as the next group (FIG. 4E). In Carrizo, ‘0460’ and ‘4580’ showed significant higher levels than some other effectors, while the rest had no difference from each other (FIG. 4F). Therefore, genotype-specific high expression of ‘4425’ in the susceptible Duncan was noted, and genotype non-specific expression of ‘3695’ and ‘0460’ in multiple citrus. The two genotypes in each HLB-response group also show effector-expression profiles that seem to be different for each other.

Differential Effector Expression Between Leaf and Root Tissues in Ca. L. asiaticus-Infected Citrus

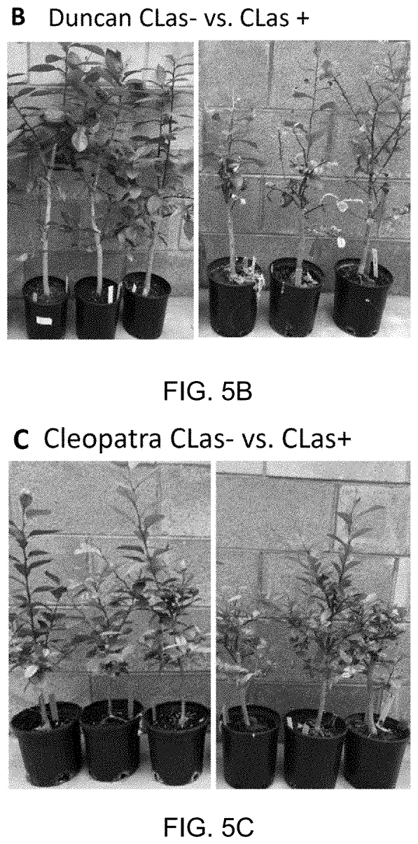

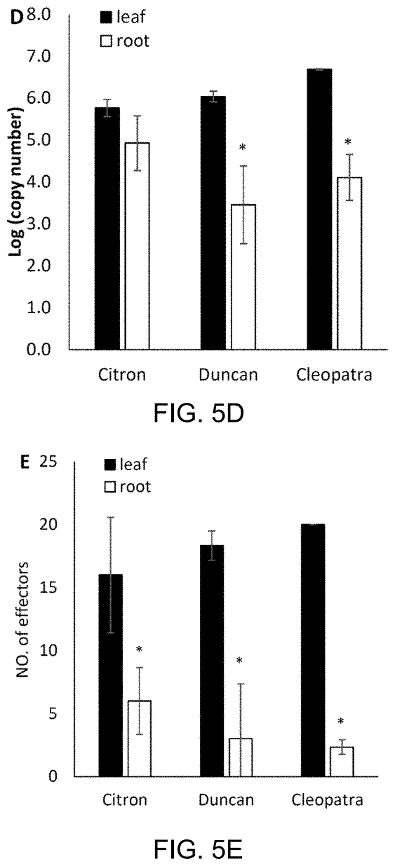

It is well known that infection of Ca. L. asiaticus occurs in citrus roots, which causes damage that directly affects tree health and also serves as source of inoculum to new foliar flushes through vascular movement of the bacterium [37]. Comparative analysis of the effector expression in leaf and root was conducted to study if effector expression has a tissue-specific pattern suggesting the bacterium employs different pathogenic strategies to colonize above- and below-ground plant organs. Ca. L. asiaticus-infected own-rooted citron, Duncan, and Cleopatra were used that had inoculated by ACP infestation in 2016 [9]. All plants were tested Ca. L. asiaticus positive in 2017 and after one year tolerant citron and Cleopatra plants were symptomatic but still maintained similar growth to their uninfected controls (FIGS. 5A and 5C), whereas Duncan plants showed stunted growth and branch dieback (FIG. 5B).

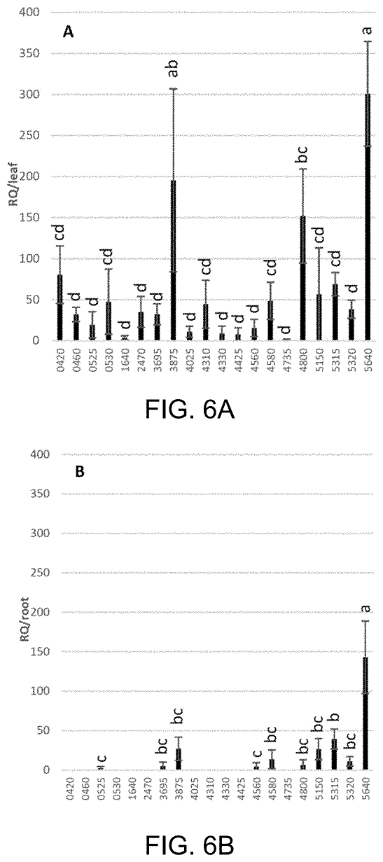

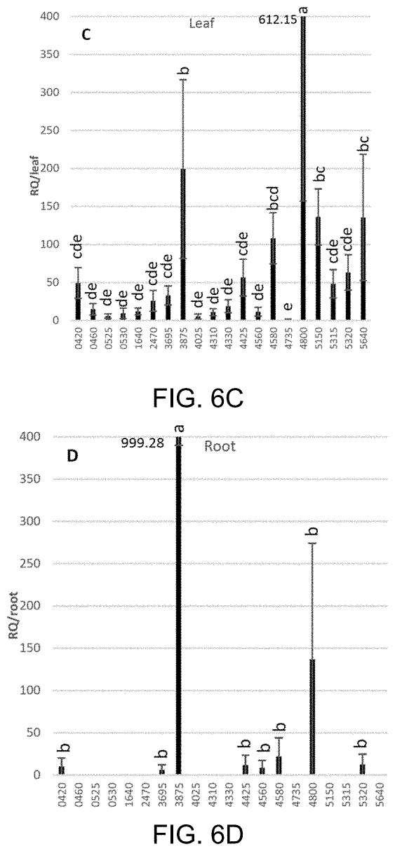

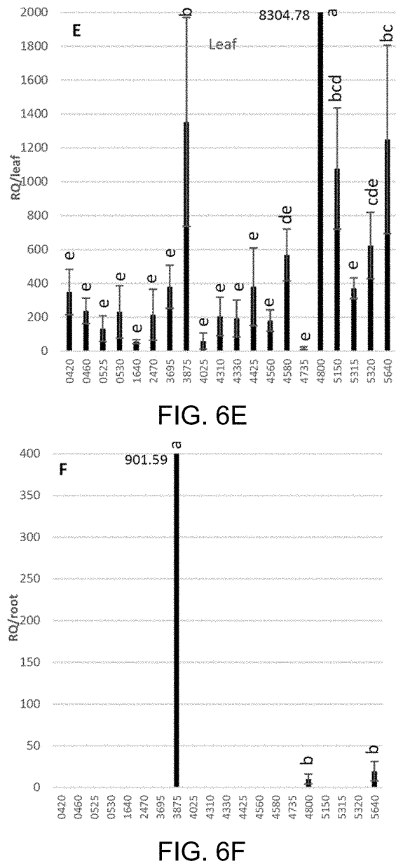

Bacterial titers determined by 16s quantification were lower in the roots of Duncan and Cleopatra than leaves, although this difference was insignificant in citron (FIG. 5D). Correspondingly, RT-qPCR analysis of effectors indicated there were lower numbers of effectors detected in roots than in leaves in all of the citrus types, which is in agreement with the previous observation on titer dependent effector mRNA detection (FIGS. 3A-3C). For the effectors with detected mRNA, in citron ‘5640’ was the highest expressed effectors in both leaf and root tissues, and the expression of ‘3875’ and ‘4900’ were also among the highest in leaves (FIG. 6A-6B). In Duncan, ‘3875’ and ‘4900’ ranked as the highest expressed effectors, although the difference was not significant for ‘4900’ in roots compared with other effectors detected (FIG. 6C-6D). The expression of Ca. L. asiaticus effector candidates were generally higher in the leaves of Cleopatra than in citron and Duncan, with top expressed candidates including ‘4900’, ‘3875’ and ‘5640’ in leaf and they were the only effectors with mRNA detected in the roots of Cleopatra (FIG. 6E-6F).

### DISCUSSION

The secretome of a pathogenic bacterium represents 287 an array of molecules that play offensive roles during colonization, among which effectors are an important class of proteins capable of suppressing defense and/or manipulating host physiology. Whole genome sequencing of Ca. L. asiaticus indicated that this intracellular fastidious bacterium lacks Type III and IV secretion systems typical for extracellular pathogens, but maintains genes encoding the general secretory pathway/Sec-translocon [3], which was proposed to be the major pathway for Ca. L. asiaticus effector secretion [10]. Due to common features such as presence of secretion SP, effectors can be predicted computationally [38] and this accelerates the discovery of key virulence factors and targeted host resistance/susceptibility genes [39]. In this study, a total of 28 effector candidates were identified, by filtering Ca. L. asiaticus genome for presence of SP, absence of transmembrane domain and relatively small protein size (see Tables 1A-1B). This list of candidates contains the effectors identified during our previous screening with different parameters [11] and was also discovered in an analysis for Sec-translocon-dependent proteins [10], confirming the commonality of bacterial effector features and reliability of the bioinformatics methods utilized in this study.

One important aspect of pathogen effectors study is to analyze the expression levels during the initial microbe-plant interactions. Effector candidates with high levels of transcript suggests an active protein utilization by the pathogen during the infection processes, which may provide guidance on the selection of effectors for further functional studies. To determine Ca. L. asiaticus expression by RT-qPCR, we used SSPs for cDNA synthesis and different sets of SSPs for qPCR (see Example 5 below), which resulted in higher specificity in target amplifications and better performance for low abundance targets than using random primers for cDNA, or the same reverse SSPs for cDNA synthesis 310 and qPCR (data not shown). As a result, a total of 20 primer sets (see Table 2) provided good Ca. L. asiaticus-specific amplification in multiple citrus genotypes including the relatively distant species Poncirus trifoliata. The expression of Ca. L. asiaticus effectors have been reported in several studies. For example, Ca. L. asiaticus effector expression profile was compared between infected citrus and ACP, and revealed interesting candidates differentially expressed in the two hosts [40]. In another study, semi-quantitative RT-qPCR detected the expression of CLIBASIA_05315, CLIBASIA_00460, CLIBASIA_03230 and CLIBASIA_05640 in several citrus types, with CLIBASIA_05315 being promising as marker gene for disease early detection as it expressed in asymptomatic tissues [41]. However, because inoculation of bacteria was performed through grafting, plants materials in these studies were from relatively late infection stages which likely were at least several months following initial disease exposure [40]. Molecular events such as defense suppression by effectors and ETI-associated hypersensitive responses occur at early stages of contact, from hours to days after initial pathogen exposure [42]. Therefore highly expressed Ca. L. asiaticus effectors during early host interactions may be especially important for bacterial virulence, and their identification may lead to discovery of citrus resistance/susceptibility genes.

To test this hypothesis, detached leaf inoculation was employed to study effector expression within 7 days of initial bacterial contact (FIGS. 2A-2D). Effective Ca. L. asiaticus inoculation was observed as soon as 6 h after infestation, resulting in a wide range of bacterial titers in multiple citrus genotypes (FIGS. 2B-2D). RT-qPCR analysis showed mRNA detection of only several effectors at various time points, which made it difficult to carry out mean comparisons for most of the genes. However, the expression of ‘4025’ in citron, and ‘5315’ in Duncan and Cleopatra were consistent at consecutive time points (see Tables 3A-3C), suggesting possible roles during early host interactions, especially ‘5315’ which manipulates host cells for starch accumulation [12] and suppresses plant defenses [13]. Across all samples effector mRNA detection was titer-dependent, with higher number of effectors amplified in samples with higher bacterial quantification. This was further confirmed by analysis with greater sample sizes in citron, Duncan and Cleopatra (FIGS. 3A-3C). This suggests that effector mRNA increases with bacterial cell number and thus effectors below the qPCR detection limit may become evident in citrus samples with high bacterial titer. It is also worth noting that the time points used in this study may not exactly correspond to ‘hours/days post inoculation (hpi/dpi)’ but rather to ‘inoculation access period (IAP)’ [36] within which inoculation occurs. This ongoing inoculation for seven days during our studies may explain ambiguous expression patterns of effectors in time point comparisons. Hence for simplicity only 7-day IAP was selected as representative of early bacterium-host interactions for the following studies.

To discover early and highly expressed effectors in citrus hosts with various HLB tolerance levels, six genotypes including citron, Duncan, Cleopatra, Pomeroy trifoliate, Washington navel, and Carrizo were subject to the detached leaf inoculation (7-day) by a common ACP population. 3 Pair-wise comparisons ranked the effector expression levels to identify higher expressed candidates in each genotype (FIG. 4A-4F). Several effectors showed relative high expression in multiple genotypes, including ‘3695’ in citron, Cleopatra and Washington navel, ‘0460’ in citron, Washington navel, Pomeroy and Carrizo, ‘0420’ in citron and Pomeroy, and ‘4580’ in Cleopatra and Carrizo (FIG. 4A-4F), suggesting these virulence factors are broadly active at the early infection stage. Among these effectors, the mRNA of ‘3695’ and ‘0460’ were detected at relatively high levels in both HLB-tolerant and susceptible citrus, suggesting they provide core virulence functions for bacterial colonization. Nevertheless, some effectors demonstrated tolerance/susceptibility-associated or host-specific high expression, including ‘0420’ in citron and Pomeroy, ‘4580’ in Cleopatra and Carrizo, ‘5320’ in Cleopatra, ‘4425’ in Duncan, ‘0525’ and ‘5315’ in Pomeroy, indicating host genetics may influence pathogen virulence factor expression. These tolerance- or resistance-associated effectors may be suitable for use as biomarkers to screen for HLB tolerance/resistance from citrus breeding materials. Further, the highly expressed effectors are good candidates for biochemical analysis to identify host binding targets, which may reveal important virulence mechanisms and lead to creation of resistant citrus.

A study was conducted using own-rooted citron, Duncan and Cleopatra plants that had been infected for more than a year, to evaluate effector expression in the pathogen after being fully established in tolerant and susceptible citrus hosts, assessing both leaf and root tissues (FIG. 5A-5E). Across all three citrus genotypes the expression patterns of effectors were similar between leaves but not roots (FIG. 6A-6F). The number of effectors detected by RT-qPCR was less in roots than in leaves (FIG. 5D-5E) which may be due to lower bacterial titers. Leaf expression of candidates ‘3875’, ‘4900’ and ‘5640’ were consistently among the highest regardless of citrus genotype/HLB tolerance levels (FIGS. 6A, 6C and 6E). The expression profiles were markedly different from those of during the early infection for citron, Duncan and Cleopatra (FIG. 4A-4C) which may suggest that Ca. L. asiaticus deploys different effectors over the time-course of infection. This is consistent with reports that this is a common strategy utilized by pathogens for successful colonization [15]. It is possible that our detached tissue-based study on early host interaction may be influenced by plant physiological changes associated with being detached from the mother plant. However, numerous researchers have employed the detached leaf inoculations to study outcomes of plant-pathogen interactions including accessing microbe pathogenicity and screening for resistant host and proved to produce consistent results to tests from whole plants [43-47]. In addition, at each sample time mRNA was also assessed for the citrus endogenous gene UPL7 and the expression was similar at all of time points and across all genotypes, suggesting metabolism was not compromised throughout the study period. In roots, high mRNA abundance of ‘5640’ in citron and ‘3875’ in Duncan and Cleopatra, even at low bacterial titers, may be evidence that their functions may contribute to HLB-related root damage such as root starch depletion and dieback reported to be causal rather than the consequence of HLB disease [37, 48]. Taken together, this part of the study identified effector candidates showing higher expression following established colonization in both HLB tolerant and susceptible citrus. The functional analysis of these candidates may enhance understanding of bacterial virulence and host interactions during chronic Ca. L. asiaticus infection.

According to the present disclosure, a group of 28 candidate effectors were identified from the Ca. L. asiaticus genome via bioinformatics. Their transcriptional levels were studied in the infected citrus leaves exposed to ACP within 7 days from susceptible Duncan grapefruit and Washington navel orange, tolerant citron and Cleopatra mandarin, and resistant Pomeroy trifoliate and Carrizo citrange. Candidate effectors CLIBASIA_03695, CLIBASIA_00460, CLIBASIA_00420, CLIBASIA_04580, CLIBASIA_05320, CLIBASIA_04425, CLIBASIA_00525 and CLIBASIA_05315 showed relatively high expression in host-specific or -nonspecific manners. In citrus with HLB exposure for over one year, the expression of effectors was compared between leaves and roots tissues and indicated relatively high expression of CLIBASIA_03875, CLIBASIA_04900 and CLIBASIA_05640 in all leaf and some root tissues of citron, Duncan and Cleopatra genotypes. The identified Ca. L. asiaticus effectors candidates with high temporal and spatial expression levels may have roles in early bacterial colonization, host tolerance suppression and long-term survival that will be subject to further function studies.

### REFERENCES

1. Sechler, A., et al., Cultivation of ‘Candidatus Liberibacter asiaticus’, ‘Ca. L. africanus’, and ‘Ca. L. americanus’ Associated with Huanglongbing. Phytopathology, 2009. 99(5): p. 480-486.
2. Parker, J. K., et al., Viability of ‘Candidatus Liberibacter asiaticus’ Prolonged by Addition of Citrus Juice to Culture Medium. Phytopathology, 2014. 104(1): p. 15-26.
3. Duan, Y. P., et al., Complete Genome Sequence of Citrus Huanglongbing Bacterium, Candidatus Liberibacter asiaticus' Obtained Through Metagenomics. Molecular Plant-Microbe Interactions, 2009. 22(8): p. 1011-1020.
4. Zheng, Z., X. Deng, and J. Chen, Whole-Genome Sequence of “Candidatus Liberibacter asiaticus” from Guangdong, China. Genome Announc, 2014. 2(2).
5. Zheng, Z., et al., Whole-Genome Sequence of “Candidatus Liberibacter asiaticus” from a Huanglongbing-Affected Citrus Tree in Central Florida. Genome Announc, 2015. 3(2).
6. Kunta, M., et al., Draft Whole-Genome Sequence of “Candidatus 467 Liberibacter asiaticus” Strain TX2351 Isolated from Asian Citrus Psyllids in Texas, USA. Genome Announc, 2017. 5(15).
7. Jones, J. D. and J. L. Dangl, The plant immune system. Nature, 2006. 444(7117): p. 323-9.
8. Zou, H. S., et al., The Destructive Citrus Pathogen, ‘Candidatus Liberibacter asiaticus’ Encodes a Functional Flagellin Characteristic of a Pathogen-Associated Molecular Pattern. Plos One, 2012. 7(9).
9. Shi, Q. C., et al., Huanglongbing tolerance-associated genes are identified by comparative transcriptomics using bacterial flagellin 22 as a proxy to challenge citrus. Journal of Citrus Pathology, 2017: p. 38.
10. Prasad, S., et al., SEC-Translocon Dependent Extracytoplasmic Proteins of Candidatus Liberibacter asiaticus. Front Microbiol, 2016. 7: p. 1989.
11. Pitino, M., et al., Transient Expression of Candidatus Liberibacter Asiaticus Effector Induces Cell Death in Nicotiana benthamiana. Frontiers in Plant Science, 2016. 7.
12. Pitino, M., V. Allen, and Y. Duan, LasDelta5315 Effector Induces Extreme Starch Accumulation and Chlorosis as Ca. Liberibacter asiaticus Infection in Nicotiana benthamiana. Front Plant Sci, 2018. 9: p. 113.
13. Clark, K., et al., An effector from the Huanglongbing-associated pathogen targets citrus proteases. Nat Commun, 2018. 9(1): p. 1718.
14. Hogenhout, S. A., et al., Emerging concepts in effector biology of plant-associated organisms. Mol Plant Microbe Interact, 2009. 22(2): p. 115-22.
15. Win, J., et al., Effector biology of plant-associated organisms: concepts and perspectives. Cold Spring Harb Symp Quant Biol, 2012. 77: p. 235-47.
16. Kourelis, J. and R. A. L. van der Hoorn, Defended to the Nines: 25 Years of Resistance Gene Cloning Identifies Nine Mechanisms for R Protein Function. Plant Cell, 2018. 30(2): p. 285-299.
17. Hein, I., et al., The zig-zag-zig in oomycete-plant interactions. Mol Plant Pathol, 2009. 10(4): p. 2 547-62.
18. Vleeshouwers, V. G., et al., Understanding and exploiting late blight resistance in the age of effectors. Annu Rev Phytopathol, 2011. 49: p. 507-31.
19. Vleeshouwers, V. G. A. A. and R. P. Oliver, Effectors as Tools in Disease Resistance Breeding Against Biotrophic, Hemibiotrophic, and Necrotrophic Plant Pathogens. Molecular Plant-Microbe Interactions, 2014. 27(3): p. 196-206.
20. Tai, T. H., et al., Expression of the Bs2 pepper gene confers resistance to bacterial spot disease in tomato. Proc Natl Acad Sci USA, 1999. 96(24): p. 14153-8.
21. Zhao, B., et al., A maize resistance gene functions against bacterial streak disease in rice. Proc Natl Acad Sci USA, 2005. 102(43): p. 15383-8.
22. van der Vossen, E. A., et al., The Rpi-blb2 gene from Solanum bulbocastanum is an Mi-1 gene homolog conferring broad-spectrum late blight resistance in potato. Plant J, 2005. 44(2): p. 208-22.
23. Yang, S., et al., Alfalfa benefits from Medicago truncatula: the RCT1 gene from M. truncatula confers broad-spectrum resistance to anthracnose in alfalfa. Proc Natl Acad Sci USA, 2008. 105(34): p. 12164-9.
24. Folimonova, S. Y., et al., Examination of the Responses of Different Genotypes of Citrus to Huanglongbing (Citrus Greening) Under Different Conditions. Phytopathology, 2009. 99(12): p. 1346-1354.
25. Cheema, S. S., S. P. Kapur, and J. S. Chohan, Evaluation of Rough Lemon Strains and Other Rootstocks against Greening-Disease of Citrus. Scientia Horticulturae, 1982. 18(1): p. 71-75.
26. Stover, E., et al., Conventional Citrus of Some Scion/Rootstock Combinations Show Field Tolerance under High Huanglongbing Disease Pressure. Hortscience, 2016. 51(2): p. 127-132.
27. Ramadugu, C., et al., Long-Term Field Evaluation Reveals Hu 515 anglongbing Resistance in Citrus Relatives. Plant Disease, 2016. 100(9): p. 1858-1869.
28. Miles, G. P., et al., Apparent Tolerance to Huanglongbing in Citrus and Citrus-related Germplasm. Hortscience, 2017. 52(1): p. 31-39.
29. Albrecht, U. and K. D. Bowman, Tolerance of the Trifoliate Citrus Hybrid US-897 (Citrus reticulata Blanco×Poncirus trifoliata L. Raf.) to Huanglongbing. HortScience, 2011. 46(1): p. 16-22.
30. Albrecht, U. and K. D. Bowman, Transcriptional response of susceptible and tolerant citrus to infection with Candidatus Liberibacter asiaticus. Plant Sci, 2012. 185-186: p. 118-30.
31. Killiny, N., et al., Metabolically speaking: Possible reasons behind the tolerance of ‘Sugar Belle’ mandarin hybrid to huanglongbing. Plant Physiol Biochem, 2017. 116: p. 36-47.
32. Nielsen, H., et al., Identification of prokaryotic and eukaryotic signal peptides and prediction of their cleavage sites. Protein Engineering, Design and Selection, 1997. 10(1): p. 1-6.
33. Sonnhammer, E. L., G. von Heijne, and A. Krogh, A hidden Markov model for predicting transmembrane helices in protein sequences. Proc Int Conf Intell Syst Mol Biol, 1998. 6: p. 175-82.
34. Krogh, A., et al., Predicting transmembrane protein topology with a hidden Markov model: application to complete genomes. J Mol Biol, 2001. 305(3): p. 567-80.
35. Petersen, T. N., et al., SignalP 4.0: discriminating signal peptides from transmembrane regions. Nat Methods, 2011. 8(10): p. 785-6.
36. Ammar el, D., A. J. Walter, and D. G. Hall, New excised-leaf assay method to test inoculativity of Asian citrus psyllid (Hemiptera: Psyllidae) with Candidatus Liberibacter asiaticus associated with citrus huanglongbing disease. J Econ Entomol, 2013. 106(1): p. 25-35.
37. Johnson, E. G., et al., Association of ‘Candidatus Liberibacter asiaticus’ root infection, but not phloem plugging with root loss on huanglongbing-affected trees prior to appearance of foliar symptoms. Plant Pathology, 2014. 63(2): p. 290-298.
38. McDermott, J. E., et al., Computational prediction of type III and IV secreted effectors in gram negative bacteria. Infect Immun, 2011. 79(1): p. 23-32.
39. Varden, F. A., et al., Taking the stage: effectors in the spotlight. Curr Opin Plant Biol, 2017. 38: p. 25-33.
40. Yan, Q., et al., Global gene expression changes in Candidatus Liberibacter asiaticus during the transmission in distinct hosts between plant and insect. Mol Plant Pathol, 2013. 14(4): p. 391-404.
41. Pagliaccia, D., et al., A Pathogen Secreted Protein as a Detection Marker for Citrus Huanglongbing. Frontiers in Microbiology, 2017. 8(2041).
42. Hann, D. R. and J. P. Rathjen, Early events in the pathogenicity of Pseudomonas syringae on Nicotiana benthamiana. Plant J, 2007. 49(4): p. 607-18.
43. Browne, R. A. and B. M. Cooke, Development and Evaluation of an in vitro Detached Leaf Assay forc Pre-Screening Resistance to Fusarium Head Blight in Wheat. European Journal of Plant Pathology, 2004. 110(1): p. 91-102.
44. Pettitt, T. R., et al., A simple detached leaf assay provides rapid and inexpensive determination of pathogenicity of Pythium isolates to ‘all year round’ (AYR) chrysanthemum roots. Plant Pathology, 2011. 60(5): p. 946-956.
45. Vleeshouwers, V. G. A. A., et al., A Laboratory Assay for Phytophthora infestans Resistance in Various Solanum Species Reflects the Field Situation. European Journal of Plant Pathology, 1999. 105(3): p. 241-250.
46. Huang, S., et al., An Accurate In Vitro Assay for High-Throughput Disease Testing of Phytophthora infestans in Potato. Plant Disease, 2005. 89(12): p. 1263-1267.
47. Arraiano, L. S., P. A. Brading, and J.562 K. M. Brown, A detached seedling leaf technique to study resistance to Mycosphaerella graminicola (anamorph Septoria tritici) in wheat. Plant Pathology, 2001. 50(3): p. 339-346.
48. Etxeberria, E., et al., Anatomical distribution of abnormally high levels of starch in HLB-affected Valencia orange trees. Physiological and Molecular Plant Pathology, 2009. 74(1): p. 76-83.

### Example 2

TABLE 1A
Predicts secretion signals using SignalP version 2.0 software
(Nielsen et al., 1997).

| | SignalPv2.01 | | SignalPv2.01 |
|---|---|---|---|
| | Ymax | SignalPv2.01 | Sprob |
| GeneID | position | Sprob Score | (Y:YES) |
| CLIBASIA_00070 | 38 | 0.976 | Y |
| CLIBASIA_00100 | 39 | 1.000 | Y |
| CLIBASIA_00195 | 14 | 0.539 | Y |
| CLIBASIA_00215 | 37 | 0.990 | Y |
| CLIBASIA_00265 | 25 | 1.000 | Y |
| CLIBASIA_00270 | 50 | 0.644 | Y |
| CLIBASIA_00400 | 23 | 0.733 | Y |
| CLIBASIA_00405 | 64 | 0.844 | Y |
| CLIBASIA_00420 | 29 | 0.999 | Y |
| CLIBASIA_00460 | 21 | 0.996 | Y |
| CLIBASIA_00470 | 20 | 0.971 | Y |
| CLIBASIA_00530 | 22 | 0.900 | Y |
| CLIBASIA_00600 | 37 | 0.616 | Y |
| CLIBASIA_00830 | 21 | 0.907 | Y |
| CLIBASIA_00880 | 55 | 0.995 | Y |
| CLIBASIA_00915 | 14 | 0.652 | Y |
| CLIBASIA_00965 | 23 | 1.000 | Y |
| CLIBASIA_00995 | 15 | 0.885 | Y |
| CLIBASIA_01020 | 25 | 0.584 | Y |
| CLIBASIA_01115 | 70 | 0.706 | Y |
| CLIBASIA_01285 | 20 | 0.908 | Y |
| CLIBASIA_01295 | 24 | 0.915 | Y |
| CLIBASIA_01300 | 32 | 0.965 | Y |
| CLIBASIA_01305 | 21 | 0.818 | Y |
| CLIBASIA_01310 | 17 | 0.543 | Y |
| CLIBASIA_01355 | 42 | 0.900 | Y |
| CLIBASIA_01400 | 32 | 0.575 | Y |
| CLIBASIA_01600 | 24 | 0.998 | Y |
| CLIBASIA_01620 | 24 | 0.515 | Y |
| CLIBASIA_01640 | 24 | 0.997 | Y |
| CLIBASIA_01675 | 17 | 0.514 | Y |
| CLIBASIA_01765 | 26 | 1.000 | Y |
| CLIBASIA_01935 | 62 | 0.716 | Y |
| CLIBASIA_02075 | 22 | 0.997 | Y |
| CLIBASIA_02110 | 24 | 0.646 | Y |
| CLIBASIA_02120 | 20 | 0.998 | Y |
| CLIBASIA_02160 | 15 | 0.963 | Y |
| CLIBASIA_02170 | 25 | 0.603 | Y |
| CLIBASIA_02175 | 23 | 0.997 | Y |
| CLIBASIA_02185 | 29 | 0.542 | Y |
| CLIBASIA_02225 | 50 | 0.992 | Y |
| CLIBASIA_02250 | 22 | 0.947 | Y |
| CLIBASIA_02275 | 46 | 0.998 | Y |
| CLIBASIA_02395 | 33 | 0.593 | Y |
| CLIBASIA_02425 | 24 | 1.000 | Y |
| CLIBASIA_02745 | 33 | 0.543 | Y |
| CLIBASIA_02790 | 32 | 0.980 | Y |
| CLIBASIA_02845 | 66 | 0.749 | Y |
| CLIBASIA_02865 | 23 | 0.985 | Y |
| CLIBASIA_02925 | 46 | 0.890 | Y |
| CLIBASIA_02935 | 29 | 0.943 | Y |
| CLIBASIA_02960 | 47 | 0.994 | Y |
| CLIBASIA_03020 | 22 | 1.000 | Y |
| CLIBASIA_03065 | 19 | 0.696 | Y |
| CLIBASIA_03070 | 25 | 0.999 | Y |
| CLIBASIA_03075 | 33 | 0.914 | Y |
| CLIBASIA_03080 | 23 | 0.991 | Y |
| CLIBASIA_03085 | 35 | 0.837 | Y |
| CLIBASIA_03120 | 24 | 0.992 | Y |
| CLIBASIA_03145 | 17 | 0.753 | Y |
| CLIBASIA_03160 | 30 | 0.688 | Y |
| CLIBASIA_03170 | 32 | 0.980 | Y |
| CLIBASIA_03230 | 23 | 1.000 | Y |
| CLIBASIA_03295 | 21 | 0.603 | Y |
| CLIBASIA_03420 | 56 | 0.810 | Y |
| CLIBASIA_03445 | 27 | 0.766 | Y |
| CLIBASIA_03450 | 41 | 0.989 | Y |
| CLIBASIA_03510 | 57 | 0.968 | Y |
| CLIBASIA_03630 | 34 | 0.682 | Y |
| CLIBASIA_03680 | 40 | 0.997 | Y |
| CLIBASIA_03685 | 39 | 0.771 | Y |
| CLIBASIA_03735 | 34 | 0.693 | Y |
| CLIBASIA_03785 | 18 | 0.784 | Y |
| CLIBASIA_03805 | 57 | 0.516 | Y |
| CLIBASIA_03875 | 28 | 0.744 | Y |
| CLIBASIA_03965 | 38 | 0.914 | Y |
| CLIBASIA_03975 | 47 | 1.000 | Y |
| CLIBASIA_04025 | 23 | 0.944 | Y |
| CLIBASIA_04035 | 43 | 0.973 | Y |
| CLIBASIA_04040 | 25 | 1.000 | Y |
| CLIBASIA_04065 | 38 | 0.976 | Y |
| CLIBASIA_04080 | 20 | 0.888 | Y |
| CLIBASIA_04120 | 23 | 0.582 | Y |
| CLIBASIA_04140 | 38 | 0.976 | Y |
| CLIBASIA_04145 | 22 | 0.808 | Y |
| CLIBASIA_04165 | 62 | 0.909 | Y |
| CLIBASIA_04170 | 29 | 0.711 | Y |
| CLIBASIA_04205 | 12 | 0.659 | Y |
| CLIBASIA_04215 | 42 | 0.658 | Y |
| CLIBASIA_04245 | 45 | 0.606 | Y |
| CLIBASIA_04260 | 22 | 0.611 | Y |
| CLIBASIA_04265 | 20 | 0.975 | Y |
| CLIBASIA_04290 | 34 | 0.996 | Y |
| CLIBASIA_04310 | 33 | 0.771 | Y |
| CLIBASIA_04320 | 23 | 0.570 | Y |
| CLIBASIA_04330 | 20 | 0.991 | Y |
| CLIBASIA_04340 | 34 | 0.897 | Y |
| CLIBASIA_04470 | 61 | 0.587 | Y |
| CLIBASIA_04520 | 21 | 0.999 | Y |
| CLIBASIA_04540 | 38 | 0.976 | Y |
| CLIBASIA_04560 | 26 | 0.991 | Y |
| CLIBASIA_04580 | 24 | 0.999 | Y |
| CLIBASIA_04665 | 22 | 0.681 | Y |
| CLIBASIA_04695 | 19 | 0.603 | Y |
| CLIBASIA_04705 | 30 | 0.993 | Y |
| CLIBASIA_04735 | 21 | 0.749 | Y |
| CLIBASIA_04750 | 22 | 0.974 | Y |
| CLIBASIA_04870 | 23 | 0.998 | Y |
| CLIBASIA_04900 | 26 | 0.970 | Y |
| CLIBASIA_04950 | 48 | 0.614 | Y |
| CLIBASIA_04995 | 24 | 0.593 | Y |
| CLIBASIA_05000 | 45 | 0.990 | Y |
| CLIBASIA_05005 | 25 | 0.956 | Y |
| CLIBASIA_05115 | 33 | 0.999 | Y |
| CLIBASIA_05150 | 35 | 1.000 | Y |
| CLIBASIA_05315 | 25 | 1.000 | Y |
| CLIBASIA_05320 | 23 | 0.999 | Y |
| CLIBASIA_05325 | 34 | 0.747 | Y |
| CLIBASIA_05480 | 69 | 1.000 | Y |
| CLIBASIA_05570 | 51 | 0.999 | Y |
| CLIBASIA_05640 | 22 | 1.000 | Y |

TABLE 1B
Predicts secretion signals using SignalP version 2.0 software

| Clas effector | | AA cleavage | NT cleavage | | |
|---|---|---|---|---|---|
| candidates | Ymax pos | position | position | Sprob | Sprob ? |
| Gene ID | SignalP2.0 | SignalP2.0 | Signalp2.01 | SignalP2.0 | SignalP2.0 |
| CLIBASIA_00420 | 29 | ALS-VV | TTGTCA-gtc | 0.999 | Y |
| CLIBASIA_00460 | 21 | ARA-QV | AGAGCT-caa | 0.996 | Y |
| CLIBASIA_00470 | 20 | AIS-GG | ATATCG-ggt | 0.971 | Y |
| CLIBASIA_00525 | 22 | DYV-YE | TATGTA-tat | 0.988 | Y |
| CLIBASIA_00530 | 22 | SCA-DD | TGTGCC-gat | 0.900 | Y |
| CLIBASIA_01640 | 24 | SFA-GF | TTTGCT-ggt | 0.997 | Y |
| CLIBASIA_02215 | 22 | NQR-VK | CAACGC-gtt | 0.949 | N |
| CLIBASIA_02470 | 20 | QSM-AL | TCTTTT-gca | 0.854 | N |
| CLIBASIA_03120 | 24 | AFS-QD | TTTTCG-caa | 0.992 | Y |
| CLIBASIA_03230 | 23 | AHA-LL | CATGCT-ctt | 1.000 | Y |
| CLIBASIA_03695 | 29 | RSH-DV | AGTCAC-gat | 0.518 | N |
| CLIBASIA_03875 | 28 | WFV-FT | TTTGTT-ttt | 0.744 | Y |
| CLIBASIA_04025 | 23 | SCG-DT | TGTGGT-gat | 0.944 | Y |
| CLIBASIA_04040 | 25 | GQA-DP | CAAGCT-gat | 1.000 | Y |
| CLIBASIA_04310 | 33 | IYS-LS | TATTCC-ctc | 0.771 | Y |
| CLIBASIA_04320 | 23 | SQA-LE | CAAGCG-tta | 0.570 | Y |
| CLIBASIA_04330 | 20 | ILA-FY | TTAGCT-ttt | 0.991 | Y |
| CLIBASIA_04425 | 20 | LNL-YD | AACCTG-tat | 0.777 | N |
| CLIBASIA_04560 | 26 | DLG-DS | CTCGGT-gat | 0.991 | Y |
| CLIBASIA_04580 | 24 | VYA-QP | TATGCG-caa | 0.999 | Y |
| CLIBASIA_04735 | 21 | SHG-CT | CATGGC-tgt | 0.749 | Y |
| CLIBASIA_04870 | 23 | VSA-SN | AGTGCA-agc | 0.998 | Y |
| CLIBASIA_04900 | 26 | SLA-VD | TTAGCA-gta | 0.970 | Y |
| CLIBASIA_05115 | 33 | ANA-SQ | AATGCT-tca | 0.999 | Y |
| CLIBASIA_05150 | 35 | AMA-DY | ATGGCA-gac | 1.000 | Y |
| CLIBASIA_05315 | 25 | ALS-GS | TTATCT-ggc | 1.000 | Y |
| CLIBASIA_05320 | 23 | ILA-AN | TTAGCT-gcc | 0.999 | Y |
| CLIBASIA_05640 | 22 | GLA-DE | TTAGCG-gat | 1.000 | Y |

### Example 3

Nucleic acid and amino acid sequences for embodiments of effectors according to the present disclosure are provided below:

1) CLIBASIA_00420
Full length nucleotide (SEQ ID NO: 1)
atgcaagctcggtgctttttatccttatcgttcctttttcccctcgttgctgccatagctgtggtttcttgtagtgccttgtcagtcg
ttgttgatagcagtgagttattgccagaaccaatggatataacaggatattgggaagatgataatggaattttatcgtattt
tcaaaaagatggaaaattcaaaaccatctctacagatggatccagtagcgttttagcaacaggctcttatcatgttaaa
attaatcaagacgtagaaattaagctcacctctttaattcgcaatacttcaggaaagattcaatgtcagttcttagattcaa
ataaactcaactgtcttgcaaaagatcaaaaacagttctatttgcgacgcacacatctaacagaattgccttcctctaaa
ccacaggacgatgtcgctgagattcctgaggatcctaccaccccttctacgtcaatcatattttataaatag
Full length amino acid (SEQ ID NO: 2)
MQARCFLSLSFLFPLVAAIAVVSCSALSVVVDSSELLPEPMDITGYWEDDNGILSYF
QKDGKFKTISTDGSSSVLATGSYHVKINQDVEIKLTSLIRNTSGKIQCQFLDSNKLNC
LAKDQKQFYLRRTHLTELPSSKPQDDVAEIPEDPTTPSTSIIFYK
Mature Protein Sequence (SEQ ID NO: 3)
VVVDSSELLPEPMDITGYWEDDNGILSYFQKDGKFKTISTDGSSSVLATGSYHVKIN
QDVEIKLTSLIRNTSGKIQCQFLDSNKLNCLAKDQKQFYLRRTHLTELPSSKPQDDV
AEIPEDPTTPSTSIIFYK
2) CLIBASIA_00460
Full length nucleotide (SEQ ID NO: 4)
atgcgtcatttgattttaataatgcttttatccatactaaccactaatatagctagagctcaagtttatcatatccattcgcctc
gtattgcaacaaaatcttcaatacatattaagtgtcatagttgcactctaaataaacatcatatcaataaaaccccttcat
cttcttccgccgtgtacaccaaaaaagaagaactaattgatgggaaaaaagcaatgatcacaacagataattttatgg
gaggtgaacctataacttttatcaaatatttatttgaagaagataaaaaatag
Full length amino acid (SEQ ID NO: 5)
MRHLILIMLLSILTTNIARAQVYHIHSPRIATKSSIHIKCHSCTLNKHHINKTPSSSSAVY
TKKEELIDGKKAMITTDNFMGGEPITFIKYLFEEDKK
Mature Protein Sequence (SEQ ID NO: 6)
QVYHIHSPRIATKSSIHIKCHSCTLNKHHINKTPSSSSAVYTKKEELIDGKKAMITTDN
FMGGEPITFIKYLFEEDKK
3) CLIBASIA_00470
Full length nucleotide (SEQ ID NO: 7)
atgtataccaaaagtttgttaatggtagcttatttgttatcttcggttgcaatatcgggtgggctatgctttaaccgcccaaaa
ggcccgtctaaagaagaacaagcacgaatagaacaaatacgagcagaagcaagagaaaggcgctaccaataa
Full length amino acid (SEQ ID NO: 8)
MYTKSLLMVAYLLSSVAISGGLCFNRPKGPSKEEQARIEQIRAEARERRYQ
Mature Protein Sequence (SEQ ID NO: 9)
GGLCFNRPKGPSKEEQARIEQIRAEARERRYQ
4) CLIBASIA_00525
Full length nucleotide (SEQ ID NO: 10)
atgaaaaaaacacaattacttttgcctcttttaacactcttaagcagttgttctgattatgtatatgaagatgcgatcaggtct
caatttgaaaatgagatcaggtattataaatcaatgcatccgtctacgcaagacgacatagaatataatctctcagaga
taaaatctttcgaaaatcaaatcctggcaatatccaataaactggaaaaaggacagaagccgaaatatctgcactta
aaagaagcgatacagaaaattgtaaaaaccattgagcaaaatgagaaagagtaa
Full length amino acid (SEQ ID NO: 11)
MKKTQLLLPLLTLLSSCSDYVYEDAIRSQFENEIRYYKSMHPSTQDDIEYNLSEIKSF
ENQILAISNKLEKGQKPKYLHLKEAIQKIVKTIEQNEKE
Mature Protein Sequence (SEQ ID NO: 12)
YEDAIRSQFENEIRYYKSMHPSTQDDIEYNLSEIKSFENQILAISNKLEKGQKPKYLH
LKEAIQKIVKTIEQNEKE
5) CLIBASIA_00530
Full length nucleotide (SEQ ID NO: 13)
atgcgctttaaaacaaaacaattaattttagctgttttagtaaccttattaggtagctgtgccgatgacaggattacggaat
taaatacccttctcgctgaatataaagaagagaatcttaaaataagagaattacaaaaggaattgtacccggctatta
gcactctcgcgcacaggatggataaattacatttcgaatcagatgctttagaccctattttgagacgtatggatggtcca
gtaaaacacgtggaaagacggattaatagtaaaaccacaacccttgaacaataa
Full length amino acid (SEQ ID NO: 14)
MRFKTKQLILAVLVTLLGSCADDRITELNTLLAEYKEENLKIRELQKELYPAISTLAHR
MDKLHFESDALDPILRRMDGPVKHVERRINSKTTTLEQ
Mature Protein Sequence (SEQ ID NO: 15)
DDRITELNTLLAEYKEENLKIRELQKELYPAISTLAHRMDKLHFESDALDPILRRMDG
PVKHVERRINSKTTTLEQ
6) CLIBASIA_01640
Full length nucleotide (SEQ ID NO: 16)
atgcttcgtagttttttgatatgtttttgctttggagctatgattttttgttcaaatgtgtcttttgctggttttcgggtttgtaatggcac
taaaaatttaatcggtgttgctgtagggtatcctgctgtaaaaggtggatggatgacggaaggatggtggcagattcctg
ggaatacttgtgaaactgttgtaaaaggtgctttgcattcacggtactattatttgtatgccgagggagtttcacatagtga
gcactgggctggaaatgtgcaaatgtgtgtagggcaagatgaatttaatatcgtggatataaagaattgttatacgcgt
ggttatttgagagttggttttactgaatacgatacagggcaacatgagaattggacagtacaactcactgagcctgcac
aagataggagataa
Full length amino acid (SEQ ID NO: 17)
MLRSFLICFCFGAMIFCSNVSFAGFRVCNGTKNLIGVAVGYPAVKGGWMTEGWWQ
IPGNTCETVVKGALHSRYYYLYAEGVSHSEHWAGNVQMCVGQDEFNIVDIKNCYT
RGYLRVGFTEYDTGQHENWTVQLTEPAQDRR
Mature Protein Sequence (SEQ ID NO: 18)
GFRVCNGTKNLIGVAVGYPAVKGGWMTEGWWQIPGNTCETVVKGALHSRYYYLY
AEGVSHSEHWAGNVQMCVGQDEFNIVDIKNCYTRGYLRVGFTEYDTGQHENWTV
QLTEPAQDRR
7) CLIBASIA_02215
Full length nucleotide (SEQ ID NO: 19)
atggaagtgttagaagcccataagctggctaaagagtatgtggaacaagctaatcaacgcgttaaagaagctgaag
aacaatctaatgcacggctacttaagggacttgggatggatgatcttgtccgatattttatgaatttggatagccaaaacc
aagcgtttttcatcgatacaatacaaaataagtatcaagatgaaatggaagaattaagtgaatttggggagaaagtat
cggattactaa
Full length amino acid (SEQ ID NO: 20)
MEVLEAHKLAKEYVEQANQRVKEAEEQSNARLLKGLGMDDLVRYFMNLDSQNQA
FFIDTIQNKYQDEMEELSEFGEKVSDY
Mature Protein Sequence (SEQ ID NO: 21)
VKEAEEQSNARLLKGLGMDDLVRYFMNLDSQNQAFFIDTIQNKYQDEMEELSEFG
EKVSDY
8) CLIBASIA_02470
Full length nucleotide (SEQ ID NO: 22)
atggctcttaattgcaacgaaactttaatgcaagccgatatgaatcaatgcacaggaaattcttttgcactagtaaaaga
gaaactagaagcaacatataaaaaagtcttagaaaaagttgaaaagcatcaaagagaattatttgaaaaatcaca
aatggcatgggaaatataccgaggttctgaatgcgcctttgctgcttctggagcagaagaaggaactgcacaatcaat
gatttatgcgaattgtctacaaggacatgccatcgaacgaaatgagaaactagaatcctaccttacatgtccagaagg
cgatctgctctgcccatttataaataattga
Full length amino acid (SEQ ID NO: 23)
MALNCNETLMQADMNQCTGNSFALVKEKLEATYKKVLEKVEKHQRELFEKSQMA
WEIYRGSECAFAASGAEEGTAQSMIYANCLQGHAIERNEKLESYLTCPEGDLLCPFI
NN
Mature Protein Sequence (SEQ ID NO: 24)
ALNCNETLMQADMNQCTGNSFALVKEKLEATYKKVLEKVEKHQRELFEKSQMAWE
IYRGSECAFAASGAEEGTAQSMIYANCLQGHAIERNEKLESYLTCPEGDLLCPFINN
9) CLIBASIA_03120
Full length nucleotide (SEQ ID NO: 25)
atggcacgcactcagatcgcattggctttatctttttttatgataactcatagttattatgcgttttcgcaagatgaaattaaga
agaataatcctacgttagaaaaaaagcccatcgtcctcatgaagcatgaaattcaagaaaaaaaaacccttgccgc
ctttacctcctttgcatcgtaa
Full length amino acid (SEQ ID NO: 26)
MARTQIALALSFFMITHSYYAFSQDEIKKNNPTLEKKPIVLMKHEIQEKKTLAAFTSFA
S
Mature Protein Sequence (SEQ ID NO: 27)
QDEIKKNNPTLEKKPIVLMKHEIQEKKTLAAFTSFAS
10)CLIBASIA_03230
Full length nucleotide (SEQ ID NO: 28)
atgctaatgaacttcagaatagcgatgttaatatcttttttagcgagtggctgtgttgcgcatgctcttcttacgaaaaagat
tgaaagtgatactgattcccgtcacgaaaaagctacgatctctctgtctgcgcatgataaggaaggatcgaaacaca
caatgaatgctgagttctcggtaccaaaaaatgatgaaaagtataccatatcttctcttacgaaaaagattgaaagtga
tactgatttccgtcgcgaaaaagctacgatctctctgtctgcccatgataaggaaggatcgaaacacacaatgaatgct
gagttctcagtaccaaaaaatgatgaaaagtataccatatctgcatgtgcgtctgacgataaaggaaacaaaagcac
gctatgtgttgagtgtccgtctccgagcactcctgggcagtatgatcttaatcattgtgccgaatgcgagaatacgacgtc
aaagggtttatgtccctga
Full length amino acid (SEQ ID NO: 29)
MLMNFRIAMLISFLASGCVAHALLTKKIESDTDSRHEKATISLSAHDKEGSKHTMNA
EFSVPKNDEKYTISSLTKKIESDTDFRREKATISLSAHDKEGSKHTMNAEFSVPKND
EKYTISACASDDKGNKSTLCVECPSPSTPGQYDLNHCAECENTTSKGLCP
Mature Protein Sequence (SEQ ID NO: 30)
LLTKKIESDTDSRHEKATISLSAHDKEGSKHTMNAEFSVPKNDEKYTISSLTKKIESD
TDFRREKATISLSAHDKEGSKHTMNAEFSVPKNDEKYTISACASDDKGNKSTLCVE
CPSPSTPGQYDLNHCAECENTTSKGLCP
11)CLIBASIA_03695
Full length nucleotide (SEQ ID NO: 31)
atgtctccactggctccactgggagatggttgcgagcagttagtaggtgcgacagttcatagtgagggaaagaaaag
aagtcacgatgtgcccagtagttcaaatcaagaagatcaaaggggaagcccttctcctaaaaaaacaaaaaatata
ttagagttattccctctaccactaccccctacccaatattatagcttctga
Full length amino acid (SEQ ID NO: 32)
MSPLAPLGDGCEQLVGATVHSEGKKRSHDVPSSSNQEDQRGSPSPKKTKNILELF
PLPLPPTQYYSF
Mature Protein Sequence (SEQ ID NO: 33)
DVPSSSNQEDQRGSPSPKKTKNILELFPLPLPPTQYYSF
12)CLIBASIA_03875
Full length nucleotide (SEQ ID NO: 34)
atggggtttcgtttttgggtatcacgtatacaaattttttttatcgctttgttgattgtattttctctctcttggtttgtttttactttgcttta
tttgattggtgatgttcgcttaaggacatgtgattttcaaatgacctttttttgtaagtaa
Full length amino acid (SEQ ID NO: 35)
MGFRFVVVSRIQIFFIALLIVFSLSWFVFTLLYLIGDVRLRTCDFQMTFFCK
Mature Protein Sequence (SEQ ID NO: 36)
FTLLYLIGDVRLRTCDFQMTFFCK
13)CLIBASIA_04025
Full length nucleotide (SEQ ID NO: 37)
atgacaatatcaaaaaatcaagccattcttttctttattacaggcatgatactttcttcttgtggtgatactctctctgactcta
agcaacataataaaatcaacaatacaaaaaatcatcttgatcttcttttcccaatagacgactcccataatcaaaagcc
tacggaaaaaaaaccaaatacatcatccataaagataaagaacaatataatagaaccacaacccggccctagtc
gctgggaaggaggatggaatggtgaaagatacgttagagaatgggaaagataa
Full length amino acid (SEQ ID NO: 38)
MTISKNQAILFFITGMILSSCGDTLSDSKQHNKINNTKNHLDLLFPIDDSHNQKPTEK
KPNTSSIKIKNNIIEPQPGPSRWEGGWNGERYVREWER
Mature Protein Sequence (SEQ ID NO: 39)
DTLSDSKQHNKINNTKNHLDLLFPIDDSHNQKPTEKKPNTSSIKIKNNIIEPQPGPSR
WEGGWNGERYVREWER
14)CLIBASIA_04040
Full length nucleotide (SEQ ID NO: 40)
atgaaaagattgaaatatcaaattattttactctctcttttgagcaccaccatggcttcttgtggacaagctgatcctgtagc
tccaccaccaccacaaacattagcagaacgtggaaaagccttattagatgaagcaacgcaaaaagcagcagaaa
aagcagcagaagcagcaagaaaagcagcagaacaagcagcagaagcagctaaaaaagcagcagaaaaaa
tcatacataaagacaagaagaaaccaaaggaaaaccaagaggtcaatgaggtaccggtagcagccaatataga
gcccgaatctcaagaaacacaacaacaagtcatcaataagacaacaacatcacaaacagacgccgaaaaaac
acctaacgaaaagcgtcaaggtacaacggatgggataaacaatcaatccaacgcaacaaatgatccatcatctaa
agacaagatagcagaaaacacaaaagaggattag
Full length amino acid (SEQ ID NO: 41)
MKRLKYQIILLSLLSTTMASCGQADPVAPPPPQTLAERGKALLDEATQKAAEKAAEA
ARKAAEQAAEAAKKAAEKIIHKDKKKPKENQEVNEVPVAANIEPESQETQQQVINKT
TTSQTDAEKTPNEKRQGTTDGINNQSNATNDPSSKDKIAENTKED
Mature Protein Sequence (SEQ ID NO: 42)
DPVAPPPPQTLAERGKALLDEATQKAAEKAAEAARKAAEQAAEAAKKAAEKIIHKDK
KKPKENQEVNEVPVAANIEPESQETQQQVINKTTTSQTDAEKTPNEKRQGTTDGIN
NQSNATNDPSSKDKIAENTKED
15)CLIBASIA_04310
Full length nucleotide (SEQ ID NO: 43)
atgaagaaaactgaaatattccgtatcttaaaagttatttggataggattgtatgtatccgtagcatcatttttcgtaactagt
ccaatttattccctctcacccgatcttataaaatatcatcaacaatcttctatgtctagtgatcttctcgatcaagaagaagt
gaggactttgaaaatttatgtagtttccacgggatctaaagcgatcgttacttttaaacgtggtagccagtataatcaaga
aggtttatcacaattaaatcgtttattgtatgattggcattccaaacaatctatagatatggatccacagttgtttgatttlitat
gggagatacaacaatatttcagtgtacccgaatatatttatatattatcaggatatcgtacgcaagaaaccaataaaat
gttgagtaggcgaaatcgtaagatagctaggaaaagtcaacatgttttggggaaagctgttgattlitatattccaggag
tttctttaaggagcctgtacaagatagctatacgtcttaaaagaggaggagttggttattattccaaatttcttcatattgatg
tgggaagagtgcgttcttggacgtga
Full length amino acid (SEQ ID NO: 44)
MKKTEIFRILKVIWIGLYVSVASFFVTSPIYSLSPDLIKYHQQSSMSSDLLDQEEVRTL
KIYVVSTGSKAIVTFKRGSQYNQEGLSQLNRLLYDWHSKQSIDMDPQLFDFLWEIQ
QYFSVPEYIYILSGYRTQETNKMLSRRNRKIARKSQHVLGKAVDFYIPGVSLRSLYKI
AIRLKRGGVGYYSKFLHIDVGRVRSWT
Mature Protein Sequence (SEQ ID NO: 45)
LSPDLIKYHQQSSMSSDLLDQEEVRTLKIYVVSTGSKAIVTFKRGSQYNQEGLSQLN
RLLYDWHSKQSIDMDPQLFDFLWEIQQYFSVPEYIYILSGYRTQETNKMLSRRNRKI
ARKSQHVLGKAVDFYIPGVSLRSLYKIAIRLKRGGVGYYSKFLHIDVGRVRSWT
16)CLIBASIA_04320
Full length nucleotide (SEQ ID NO: 46)
atggtacgtgttttttgtgcaataatctttgtattaattacgtttattggggaattttcccaagcgttagagcatgaggatgaatt
aaaggtcaactttggtctcatgcgacgagtaatgattgatttatggagccgtgaaatctcttcatatagaacacctctttctt
tagatttggattataaacacagggtttatctggatacatacaaaagctttagcataaatctcggatttgaaacatttaatga
aatagttaacccaactacgatgagagtgttggttctgcctgtgttatctatgcataaaacatggaataataattttgatgatt
cctatttttttaagaaaataggagtcggtgtcgtagcatctactgggttcaatacaggcgataagtggcttggagcagag
atgggaatgtcattttacgtatacccgacaccgtggcttatattacagagcgattttgcgattcgtcacgccagtagcgat
gttgttgtatgtatgcgttatcaagcgaaatttctgataactgatagtataggtattctttatcgaaatgtatcagcagtgtca
gcggcagtagataagaatataggtttaggcgttactaagataggtcttgattatgtttataaattctaa
Full length amino acid (SEQ ID NO: 47)
MVRVFCAIIFVLITFIGEFSQALEHEDELKVNFGLMRRVMIDLWSREISSYRTPLSLDL
DYKHRVYLDTYKSFSINLGFETFNEIVNPTTMRVLVLPVLSMHKTWNNNFDDSYFFK
KIGVGVVASTGFNTGDKWLGAEMGMSFYVYPTPWLILQSDFAIRHASSDVVVCMR
YQAKFLITDSIGILYRNVSAVSAAVDKNIGLGVTKIGLDYVYKF
Mature Protein Sequence (SEQ ID NO: 48)
LEHEDELKVNFGLMRRVMIDLWSREISSYRTPLSLDLDYKHRVYLDTYKSFSINLGF
ETFNEIVNPTTMRVLVLPVLSMHKTWNNNFDDSYFFKKIGVGVVASTGFNTGDKWL
GAEMGMSFYVYPTPWLILQSDFAIRHASSDVVVCMRYQAKFLITDSIGILYRNVSAV
SAAVDKNIGLGVTKIGLDYVYKF
17)CLIBASIA_04330
Full length nucleotide (SEQ ID NO: 49)
atgaaatataagatcgcgattatcatattattggttttggttggagtgattttagctttttattttcaacagagcagtactccac
aaaaccatcttctctttttttctgaaaaagcggtatggaaaggagattcggaaacatacttccaatgtaaacgagcacat
gaattcgatgagaactttgacaactgtattatcacaagtattaaaaaaacgggaggaacccaagaagcgttacgag
cggctcaatatctggaaaaatacttagagcctggatacgtatcttcatatcgtaaggaagctttaattggtatcgtagag
gtgcaatatccttatagagctaatgagaatagcgggactttgcttattcctaccgtgggaagccatattattgatatcaatg
attctagtgttcatcagctttacgattcttctcctatagcaaaggattttgtattaagaaatccaggtgttttcccttatagtgcg
ggacatttcgttaaaagcagtcataaagatggattaatagaattaattttttcttatcctttgagaagttgtcatggttgtgaa
gatattggttttatggatattgcatataaatttacaactaaaggtgcattcattggtagaaaagtatttggtattcggaatgat
aatgcaaaatatcctatgcacttctttatataa
Full length amino acid (SEQ ID NO: 50)
MKYKIAIIILLVLVGVILAFYFQQSSTPQNHLLFFSEKAVWKGDSETYFQCKRAHEFD
ENFDNCIITSIKKTGGTQEALRAAQYLEKYLEPGYVSSYRKEALIGIVEVQYPYRANE
NSGTLLIPTVGSHIIDINDSSVHQLYDSSPIAKDFVLRNPGVFPYSAGHFVKSSHKDG
LIELIFSYPLRSCHGCEDIGFMDIAYKFTTKGAFIGRKVFGIRNDNAKYPMHFFI
Mature Protein Sequence (SEQ ID NO: 51)
FYFQQSSTPQNHLLFFSEKAVWKGDSETYFQCKRAHEFDENFDNCIITSIKKTGGT
QEALRAAQYLEKYLEPGYVSSYRKEALIGIVEVQYPYRANENSGTLLIPTVGSHIIDIN
DSSVHQLYDSSPIAKDFVLRNPGVFPYSAGHFVKSSHKDGLIELIFSYPLRSCHGCE
DIGFMDIAYKFTTKGAFIGRKVFGIRNDNAKYPMHFFI
18)CLIBASIA_04425
Full length nucleotide (SEQ ID NO: 52)
atgaagaagtatatcacattattaacagtattactcataagtaacgtgctgaacctgtatgatgcgaaagcaagaagat
tcccaacctatggatctgaagaacgtatagcaacgtgcgcaaaaccgggctattcttcacgattagcacaactatgtg
cagaaaacgaaaaaagacttaaagaattcgacaaaataacaagagaattgaatacgttatcagaaaacgaaaaa
aaagcattctttgaacacgagaaaaaagtaacgagcaatctaaactacaacgcaagagacagaaagcataatat
aaatcaattctacgaagcgagaggaaagtaccgctacggaaatggatattatcgaaactaccgatcccagtaa
Full length amino acid (SEQ ID NO: 53)
MKKYITLLTVLLISNVLNLYDAKARRFPTYGSEERIATCAKPGYSSRLAQLCAENEKR
LKEFDKITRELNTLSENEKKAFFEHEKKVTSNLNYNARDRKHNINQFYEARGKYRY
GNGYYRNYRSQ
Mature Protein Sequence (SEQ ID NO: 54)
YDAKARRFPTYGSEERIATCAKPGYSSRLAQLCAENEKRLKEFDKITRELNTLSENE
KKAFFEHEKKVTSNLNYNARDRKHNINQFYEARGKYRYGNGYYRNYRSQ
19)CLIBASIA_04560
Full length nucleotide (SEQ ID NO: 55)
atgaaatcaaaaaatattctcattgtatcaacgttagtcatctgcgttctatctattagtagttgtgacctcggtgattccattg
caaaaaaaagaaatacaataggtaacacgatcaagaagtccataaatagagttatacaagagaataataaacctc
gaaatatgactatatttaaaacagaagttaagagagatatacgtcgtgctagcaggctatctttggaagagaaatcca
aaaatgcagataaacctactgtgatagagaatcaagctgataatatcaacattgaggtagaagtcgctactaatctga
acccaaaccatcaagctagtgagatcgatattgcaatagaaaacctgcctgatttgaaatcaaaccatcaagctagt
gagatcgatattgcaatagaaaacctgcctgatttgaaatcaaaccatcaagctagtgagatcgatattgcaatagaa
aacctgcctgatcatcaagttgatagaaatcataccctcagcaacctcagaggtgcttgttatcagccctctcttgtgtct
aactcgtcgttaaagctatgggacgtagcattttaa
Full length amino acid (SEQ ID NO: 56)
MKSKNILIVSTLVICVLSISSCDLGDSIAKKRNTIGNTIKKSINRVIQENNKPRNMTIFKT
EVKRDIRRASRLSLEEKSKNADKPTVIENQADNINIEVEVATNLNPNHQASEIDIAIEN
LPDLKSNHQASEIDIAIENLPDLKSNHQASEIDIAIENLPDHQVDRNHTLSNLRGACY
QPSLVSNSSLKLWDVAF
Mature Protein Sequence (SEQ ID NO: 57)
DSIAKKRNTIGNTIKKSINRVIQENNKPRNMTIFKTEVKRDIRRASRLSLEEKSKNADK
PTVIENQADNINIEVEVATNLNPNHQASEIDIAIENLPDLKSNHQASEIDIAIENLPDLK
SNHQASEIDIAIENLPDHQVDRNHTLSNLRGACYQPSLVSNSSLKLWDVAF
20) CLIBASIA_04580
Full length nucleotide (SEQ ID NO: 58)
atgttttggattgcaaaaaaatttttttggatatcagtgttattaatcgttctgtctaatgtatatgcgcaaccttttttggaagag
acggaaaaaggtaagaaaaccgaaatcacggattttatgactgccacaagtggtactgtgggttatgcgagcaatct
ttgtaatgcaaaaccagaaatatgtcttttgtggaaaaagattatgcgtaatgttaaaagacataccttaaatggagcca
agattgtatatggttttgcgaaatcggctcttgagaaaaatgaaagagagagtgtagctatacattccaagaatgaata
tccacctcctttgccgtcgcatcattag
Full length amino acid (SEQ ID NO: 59)
MFWIAKKFFWISVLLIVLSNVYAQPFLEETEKGKKTEITDFMTATSGTVGYASNLCNA
KPEICLLWKKIMRNVKRHTLNGAKIVYGFAKSALEKNERESVAIHSKNEYPPPLPSH
H
Mature Protein Sequence (SEQ ID NO: 60)
QPFLEETEKGKKTEITDFMTATSGTVGYASNLCNAKPEICLLWKKIMRNVKRHTLNG
AKIVYGFAKSALEKNERESVAIHSKNEYPPPLPSHH
21)CLIBASIA_04735
Full length nucleotide (SEQ ID NO: 61)
atgcatttttatcgttttattctcttaaatctttacatgctcacattattttcacatggctgtacacaaatagatttcggaaatatttt
tttcaaaaaaccagagatctcccttcctccttctgttgaatcagagattcttcttccgcctattcctgaagaagaatttgatc
aggacgatatttctgtgcctagtaaggataataatgccattaggatgggaataataggtgcttggaaagtatcataccg
agatgtcgactgtaagatgattttgacattgactcgatttaaaaagaattttcgtggaaccgctcgaagttgccatggtag
gttagcatcattagcagcatggaatataatagatgaggatagttttgagcttaaaaataaatccggtcaaactatcattgt
tttctataaaactgcggaacagtctttcgagggatcttttcagggtgaaagtgataaagttataatttctcggtag
Full length amino acid (SEQ ID NO: 62)
MHFYRFILLNLYMLTLFSHGCTQIDFGNIFFKKPEISLPPSVESEILLPPIPEEEFDQD
DISVPSKDNNAIRMGIIGAWKVSYRDVDCKMILTLTRFKKNFRGTARSCHGRLASLA
AWNIIDEDSFELKNKSGQTIIVFYKTAEQSFEGSFQGESDKVIISR
Mature Protein Sequence (SEQ ID NO: 63)
CTQIDFGNIFFKKPEISLPPSVESEILLPPIPEEEFDQDDISVPSKDNNAIRMGIIGAWK
VSYRDVDCKMILTLTRFKKNFRGTARSCHGRLASLAAWNIIDEDSFELKNKSGQTIIV
FYKTAEQSFEGSFQGESDKVIISR
22)CLIBASIA_04870
Full length nucleotide (SEQ ID NO: 64)
atgaatcgcgtatttgcgtatatattgttctgtttcctaggattgatggggtattctgttagtgcaagcaacaatgatacctct
aaaataccagatgctaagtttggcagttttttacaaatcagatccaaagaatccgtcattaataaagagttcgtaaccaa
ggtggaagagctatacgaaaaagcccaaaaagcacataaaaaacgggataaggtatatggtgcttacgataaagt
atcaagccataaaaaatcgcctaaagaattgagtaaagctttttacatagacttcagaacagaactaaaatattttaaa
gcactaactaaatattacaaatcagttgtagcagaactcagagaatttggtttaggcaaatccgcaatagaaattgag
gaaatcactaaagccgtcgacacactcacaagagcgtataacgaatacaaaaaagaaataagagagttaataga
agagtttattgaattgggattcgaccagtgcgatgagtgtgatttgtgtagtgaaaaagcagatgtaatacaaaaaaaa
aggatagcatttgaaatggtagaacgtgaattcgcggaaaaattagaaggtaaattcgtaagaaaataa
Full length amino acid (SEQ ID NO: 65)
MNRVFAYILFCFLGLMGYSVSASNNDTSKIPDAKFGSFLQIRSKESVINKEFVTKVEE
LYEKAQKAHKKRDKVYGAYDKVSSHKKSPKELSKAFYIDFRTELKYFKALTKYYKSV
VAELREFGLGKSAIEIEEITKAVDTLTRAYNEYKKEIRELIEEFIELGFDQCDECDLCS
EKADVIQKKRIAFEMVEREFAEKLEGKFVRK
Mature Protein Sequence (SEQ ID NO: 66)
SNNDTSKIPDAKFGSFLQIRSKESVINKEFVTKVEELYEKAQKAHKKRDKVYGAYDK
VSSHKKSPKELSKAFYIDFRTELKYFKALTKYYKSVVAELREFGLGKSAIEIEEITKAV
DTLTRAYNEYKKEIRELIEEFIELGFDQCDECDLCSEKADVIQKKRIAFEMVEREFAE
KLEGKFVRK
23)CLIBASIA_04900
Full length nucleotide (SEC) ID NO: 67)
atgaattttaatgggtatggtgcactttttttcgtagtatttttaagtattgtagtaccgaatcattcgttagcagtagatctttatc
tgcctaggaaaattgacttatttaatgaggcagataacaatgtggaatatcaggatgatgaatatggtatatggtctggt
aattatgtaggattgcatatatctcgtttatatgaaacgcaccccttagctgatactatcaataggaaaacgtataatagtt
tactaccaaatggattgggaattgaattaggacataatatacagctagaagattttgtttttggtatcaattgtcatactact
gctgcgaaggatgattctacgttttatcgtctaaaagaaaaatattttatttatggggatgttgtactaaaagcaggatattc
tgtggattctcttcttatctatggaatgggaggatttggaggagcatacgttatagattcaagccttgagaaggttgaatca
gacaacagtaaaaatgctaaaggtagatttgatgggcatggatcaagtgtagtattaggtataggtttagattatatggt
aaattacgacatctctttatctgctagttatcgttatattcctcatcacattcattctgttaataattctaacgcaaaaagtgat
gttgaaagagtggacaggaaaggtaatgcccacatcgcatctcttggtataaatatgcacttctaa
Full length amino acid (SEQ ID NO: 68)
MNFNGYGALFFVVFLSIVVPNHSLAVDLYLPRKIDLFNEADNNVEYQDDEYGIWSG
NYVGLHISRLYETHPLADTINRKTYNSLLPNGLGIELGHNIQLEDFVFGINCHTTAAK
DDSTFYRLKEKYFIYGDVVLKAGYSVDSLLIYGMGGFGGAYVIDSSLEKVESDNSKN
AKGRFDGHGSSVVLGIGLDYMVNYDISLSASYRYIPHHIHSVNNSNAKSDVERVDR
KGNAHIASLGINMHF
Mature Protein Sequence (SEQ ID NO: 69)
VDLYLPRKIDLFNEADNNVEYQDDEYGIWSGNYVGLHISRLYETHPLADTINRKTYN
SLLPNGLGIELGHNIQLEDFVFGINCHTTAAKDDSTFYRLKEKYFIYGDVVLKAGYSV
DSLLIYGMGGFGGAYVIDSSLEKVESDNSKNAKGRFDGHGSSVVLGIGLDYMVNYD
ISLSASYRYIPHHIHSVNNSNAKSDVERVDRKGNAHIASLGINMHF
24)CLIBASIA_05115
Full length nucleotide (SEC) ID NO: 70)
atgttcttaaatgttctaaaagatttttttgttcctaggatacgatttttgattgtattaatggtaagcagtgtatccgctgggtat
gcgaatgcttcacaacctgagcctacattacgtaatcaattttccagatggtctgtatatgtatatccagatttaaataaaa
aactttgtttttcactttctgttcctgttacggtagaaccgttagaaggtgttagacatggggttaatttctttattatttcattgaa
aaaagaggaaaattctgcttatgtttcggaattagttatggattatcctttagatgaagaagagatggtttcgcttgaagta
aaaggaaaaaatgctagcggaacaatatttaaaatgaagtcttataataatagagctgcattcgaaaaaagatctca
agatactgttcttattgaggagatgaaacggggaaaagaattagttgtatccgccaaatctaaacgtggaacaaatac
ccgctatatctattctctcattggattatctgattctttggcagatattcgtaaatgtaattaa
Full length amino acid (SEQ ID NO: 71)
MFLNVLKDFFVPRIRFLIVLMVSSVSAGYANASQPEPTLRNQFSRWSVYVYPDLNK
KLCFSLSVPVTVEPLEGVRHGVNFFIISLKKEENSAYVSELVMDYPLDEEEMVSLEV
KGKNASGTIFKMKSYNNRAAFEKRSQDTVLIEEMKRGKELVVSAKSKRGTNTRYIY
SLIGLSDSLADIRKCN
Mature Protein Sequence (SEQ ID NO: 72)
SQPEPTLRNQFSRWSVYVYPDLNKKLCFSLSVPVTVEPLEGVRHGVNFFIISLKKEE
NSAYVSELVMDYPLDEEEMVSLEVKGKNASGTIFKMKSYNNRAAFEKRSQDTVLIE
EMKRGKELVVSAKSKRGTNTRYIYSLIGLSDSLADIRKCN
25)CLIBASIA_05150
Full length nucleotide (SEQ ID NO: 73)
atgatgagagatataagaaaaattagaaattattttaggaatactgctaaaattatattgagtgggttatttctagggtttttt
tcttctgctgcaatggcagactatgggtattctccccagtttcagccgactataatggtgtccaattttgcaaaatttaaag
ggttatatgttgctgctgatttttccaaaatagatcatcagtcgcctgttcgtttgcaaaatctttctttaaatggggtgtccatt
ggtcttgatggtcaagatggaacccttgtttatggtgcttctttgggtgtcgagggatttcatcttgaaccacgaggggga
attgatggggataaggtagcgggaacactcttgtttcgtaccggttttacgtttgataataataattcttctattctccaaaat
actcttatttatgggtttggtggagctcgtataagaaatattatgtctgttgaatctgctgacacagcaaaatccacaatac
gaaacattgtagcaaacggttttttagataaagttattggtgtggggattgaaaagaaacttgctagcatgctctcgattc
gtggtgagtatcgttatgtcgcttgttatgaccagccttgggatgtcagcaagtggagagaaaaaggtgacttcacagc
tggtgtggttttacgcttttaa
Full length amino acid (SEQ ID NO: 74)
MMRDIRKIRNYFRNTAKIILSGLFLGFFSSAAMADYGYSPQFQPTIMVSNFAKFKGL
YVAADFSKIDHQSPVRLQNLSLNGVSIGLDGQDGTLVYGASLGVEGFHLEPRGGID
GDKVAGTLLFRTGFTFDNNNSSILQNTLIYGFGGARIRNIMSVESADTAKSTIRNIVA
NGFLDKVIGVGIEKKLASMLSIRGEYRYVACYDQPWDVSKWREKGDFTAGVVLRF
Mature Protein Sequence (SEQ ID NO: 75)
DYGYSPQFQPTIMVSNFAKFKGLYVAADFSKIDHQSPVRLQNLSLNGVSIGLDGQD
GTLVYGASLGVEGFHLEPRGGIDGDKVAGTLLFRTGFTFDNNNSSILQNTLIYGFGG
ARIRNIMSVESADTAKSTIRNIVANGFLDKVIGVGIEKKLASMLSIRGEYRYVACYDQ
PWDVSKWREKGDFTAGVVLRF
26)CLIBASIA_05315
Full length nucleotide (SEQ ID NO: 76)
gtgcgtaaaaatttattaacctcaacctcatctttaatgttttttttcttatcttctggctatgctttatctggcagtagttttggttgtt
gtggagaatttaaaaagaaagcttcttcacctagaatccatatgcgtcctttcaccaagtcatcaccttataacaactca
gtgagtaatacagtgaataatactccgcgtgttcctgatgtctctgaaatgaacagctctaggggttctgctcctcaatctc
atgttaatgtttcttctcctcattataaacatgaatacagttcttcttcggcatcttcttcaacacatgcttcgcctcctcctcattt
tgaacagaagcacattagtcgcactcgtattgactcaagccctccacccggtcatattgatcctcatcccgatcatatta
gaaatacacttgcactccatagaaaaatgttggagcagtcttga
Full length amino acid (SEQ ID NO: 77)
MRKNLLTSTSSLMFFFLSSGYALSGSSFGCCGEFKKKASSPRIHMRPFTKSSPYNN
SVSNTVNNTPRVPDVSEMNSSRGSAPQSHVNVSSPHYKHEYSSSSASSSTHASPP
PHFEQKHISRTRIDSSPPPGHIDPHPDHIRNTLALHRKMLEQS
Mature Protein Sequence (SEQ ID NO: 78)
GSSFGCCGEFKKKASSPRIHMRPFTKSSPYNNSVSNTVNNTPRVPDVSEMNSSRG
SAPQSHVNVSSPHYKHEYSSSSASSSTHASPPPHFEQKHISRTRIDSSPPPGHIDP
HPDHIRNTLALHRKMLEQS
27)CLIBASIA_05320
Full length nucleotide (SEQ ID NO: 79)
atgagtaagtttgtggtgaggattatgtttttattaagtgctatatcttcgaatcctatcttagctgccaatgagcactcttctgt
atcggaacagaagagaaaggagacaacagtaggatttatcagtcgtcttgtcaataaacgtcctgtcgctaataaac
gttgtcctaatgcgactaaacaaacaccacccgatcatggatccaagtacgatacacgagaggtgcttatgctctttgg
aggcttaaacaattga
Full length amino acid (SEQ ID NO: 80)
MSKFVVRIMFLLSAISSNPILAANEHSSVSEQKRKETTVGFISRLVNKRPVANKRCP
NATKQTPPDHGSKYDTREVLMLFGGLNN
Mature Protein Sequence (SEQ ID NO: 81)
ANEHSSVSEQKRKETTVGFISRLVNKRPVANKRCPNATKQTPPDHGSKYDTREVL
MLFGGLNN
28)CLIBASIA_05640
Full length nucleotide (SEQ ID NO: 82)
atgactattaagaaagtactaattgcttcaactttattatccctctgtggctgtggtttagcggatgaaccaaagaagctga
atcctgatcaactctgtgatgccgtttgtaggcttactttagaagaacaaaaagagttacaaactaaggtaaatcagag
gtatgaagaacaccttacaaagggtgcgaaactatctagtgattaa
Full length amino acid (SEQ ID NO: 83)
MTIKKVLIASTLLSLCGCGLADEPKKLNPDQLCDAVCRLTLEEQKELQTKVNQRYEE
HLTKGAKLSSD
Mature Protein Sequence (SEQ ID NO: 84)
DEPKKLNPDQLCDAVCRLTLEEQKELQTKVNQRYEEHLTKGAKLSSD

### Example 4

TABLE 2
Primer sequences for reverse transcription and qPCR analysis of
effector expression
Embodiments of Primers for detection of effectors

| Gene focus | SEQ ID NO: | Reverse transcription | SEQ ID NO: | Forward (5′-3′) | SEQ ID NO: | Reverse (5′-3′) |
|---|---|---|---|---|---|---|
| CLIBASIA_00420 | 85 | CTGGATCCATCTGTAGAGATGG | 86 | CGGTGCTTTTTATCCTTATCGTTCCTTTTT | 87 | GGCAATAACTCACTGCTATCAACAACGACT |
| CLIBASIA_00460 | 88 | ATAAAAGTTATAGGTTCACTCCCATA | 89 | GCCTCGTATTGCAACAAAATC | 90 | ACGGCGGAAGAAGATGAAG |
| CLIBASIA_00525 | 91 | ACAATTTTCTGTATCGC | 92 | TTATAAATCAATGCATCCGTCTACGCAAGA | 93 | TATTTCGGCTTCTGTCCTTTTTCCAGTTTA |
| CLIBASIA_00530 | 94 | TGGACCATCCATACGTCTCA | 95 | CTGTGCCGATGACAGGATTA | 96 | AGCCGGGTACAATTCCTTTT |
| CLIBASIA_01640 | 97 | CTCATGTTGCCCTGTATCG | 98 | GTTGCTGTAGGGTATCCTGCTGTAAAAGGT | 99 | CCAACTCTCAAATAACCACGCGTATAACAA |
| CLIBASIA_02470 | 100 | GCTCCAGAAGCAGCAAAGG | 101 | AATCAATGGCTCTTAATTGCAACGAAACTT | 102 | TATATTTCCCATGCCATTTGTGATTTTTCA |
| CLIBASIA_03695 | 103 | GGCTTCCCCTTTGATCTTCT | 104 | AAGCGCCATCCTACCCTACT | 105 | GGGCACATCGTGACTTCTTT |
| CLIBASIA_03875 | 106 | TCCTTAAGCGAACATCACCA | 107 | TTTCGTTTTTGGGTATCACG | 108 | GCAAAGTAAAAACAAACCAAGAGA |
| CLIBASIA_04025 | 109 | ATCTTTCCCATTCTCTAACG | 110 | TCTTCTTTTCCCAATAGACGACTCCCATAA | 111 | CCCATTCTCTAACGTATCTTTCACCATTCC |
| CLIBASIA_04310 | 112 | CGTCCAAGAACGCACTCTTC | 113 | CATGTTTTGGGGAAAGCTGTTGATTTTTAT | 114 | TCTTCCCACATCAATATGAAGAAATTTGGA |
| CLIBASIA_04330 | 115 | GTAACGCTTCTTGGGTTCC | 116 | CAGAGCAGTACTCCACAAAACCATCTTCTC | 117 | AGTTCTCATCGAATTCATGTGCTCGTTTAC |
| CLIBASIA_04425 | 118 | TCCGTAGCGGTACTTTCCTC | 119 | CTCATAAGTAACGTGCTGAACCTGT | 120 | GTTGCTATACGTTCTTCAGATCCAT |
| CLIBASIA_04560 | 121 | GGGCTGATAACAAGCACCTC | 122 | TGCTAGCAGGCTATCTTTGGA | 123 | GCGACTTCTACCTCAATGTTGATA |
| CLIBASIA_04580 | 124 | CTCAAGAGCCGATTTCGC | 125 | TCGTTCTGTCTAATGTATATGCGCAACCTT | 126 | GCAAAACCATATACAATCTTGGCTCCATTT |
| CLIBASIA_04735 | 127 | GTCAAAATCATCTTACAGTCG | 128 | CTTCTTCCGCCTATTCCTGAAGAAGAATTT | 129 | TTCCAAGCACCTATTATTCCCATCCTAATG |
| CLIBASIA_04800 | 130 | TGTAGCTCCTTCTGCACGTC | 131 | TCGCAGTAGCTGATTTCGTG | 132 | TACATTCCTCAGCGGCTTTT |
| CLIBASIA_05150 | 133 | CGAATCGAGAGCATGCTAGC | 134 | ACACTCTTGTTTCGTACCGGTTTTACGTTT | 135 | AAACCGTTTGCTACAATGTTTCGTATTGTG |
| CLIBASIA_05315 | 136 | CGGGTGGAGGGCTTGAGTC | 137 | CCTGATGTCTCTGAAATGAACAGCTCTAGG | 138 | GAGTGCGACTAATGTGCTTCTGTTCAAAAT |
| CLIBASIA_05320 | 139 | ATGATCGGGTGGTGTTTGTT | 140 | TCTTAGCTGCCAATGAGCAC | 141 | AGCGACAGGACGTTTATTGA |
| CLIBASIA_05640 | 142 | TCACTAGATAGTTTCGCACC | 143 | GCTTCAACTTTATTATCCCTCTGTGGCTGT | 144 | GATAGTTTCGCACCCTTTGTAAGGTGTTCT |
| 16s las-Long | 145 | TCCCTATAAAGTACCCAACACTAGGTAAA | 146 | CTTACCAGCCCTTGACATGTATAGGA | 147 | TCCCTATAAAGTACCCAACACTAGGTAAA |
| UPL7 | 149 | TCAGGAACA GCAAAAGCAAG | 149 | CAAAGAAGTGCAGCGAGAGA | 150 | TCAGGAACA GCAAAAGCAAG |

### Example 5

The design of primers for reverse transcription (RT) and qPCR amplifying Ca. L. asiaticus effector candidates. The putative effector sequences were obtained from the Ca. L. asiaticus psy62 genomic database. CLIBASIA_05315 is provided as an example, where the RT primer (italic) was selected towards the 3′ end of the sense strand (reverse complement to the selected), and the forward (bold) and reverse (bold italic, reverse complement to the selected) primers for qPCR were located downstream along the cDNA synthesis direction.

```
>CLIBASIA_05315
(SEQ ID NO: 76)
GTGCGTAAAAATTTATTAACCTCAACCTCATCTTTAATGTTTTTTTTCTTATCTTCTGGCT
ATGCTTTATCTGGCAGTA
GTTTTGGTTGTTGTGGAGAATTTAAAAAGAAAGCTTCTTCACCTAGAATCCATATGCGTC
CTTTCACCAAGTCATCA
                                                                                                  Forward
CCTTATAACAACTCAGTGAGTAATACAGTGAATAATACTCCGCGTGTTCCTGATGTCTCT
GAAATGAACAGCTCT
AGGGGTTCTGCTCCTCAATCTCATGTTAATGTTTCTTCTCCTCATTATAAACATGAATAC
AGTTCTTCTTCGGCATCTT
                                                                                            RT direction
                                                        Reverse [symbol]
CTTCAACACATGCTTCGCCTCCTCCTC [symbol] [symbol]
ATTGACTCAAGCCCTC
ACCCGGTCATATTGATCCTCATCCCGATCATATTAGAAATACACTTGCACTCCATAGAAA
AATGTTGGAGCAGTCTT
GA
```

### Example 6

A time course study on the expression of Ca. L. asiaticus effectors in citron (Table 3A), Duncan (Table 3B), and Cleopatra (Table 3C) using the detached leaf assay. Three randomly selected Ca. L. asiaticus-infected samples were collected for each time point (FIGS. 2A-2D) and were analyzed for expression by RT-PCR. The data was presented as relative expression levels, based on normalization with citrus UPL7 (delta Ct), and transformed into 2-ΔCt. The table was formatted with color scales using Excel where values increase from green to red colors. The two highlighted columns had were the two samples with high bacterial titer and larger number of detected candidate effectors.

TABLE 3A

| | citron | | | | | | | | | | | |
|---|---|---|---|---|---|---|---|---|---|---|---|---|
| Genes | 6 h | 6 h | 6 h | 24 h | 24 h | 24 h | 3 d | 3 d | 3 d | 7 d | 7 d | 7 d |
| CLIBASIA_00420 | | | | | | | | 9.83 | | | | |
| CLIBASIA_00460 | 131.6 | 9.137 | 4.847 | 6.469 | | | 4.944 | 682.4 | | | | |
| CLIBASIA_00525 | | | 0.43 | | | | | | | | | |
| CLIBASIA_00530 | | | | | 6.471 | | | 10.26 | 6.043 | | | |
| CLIBASIA_01640 | | | | | | | | | | | | |
| CLIBASIA_02470 | | | | | | | | 9.337 | | | | |
| CLIBASIA_03695 | | | | 6.897 | | | | 174.8 | 61.66 | | 3.877 | 14.57 |
| CLIBASIA_03875 | | | | | | | | 10.57 | | | | |
| CLIBASIA_04025 | 0.641 | 0.748 | 0.447 | 0.318 | 0.209 | | 0.161 | 0.546 | 0.79 | | 0.455 | |
| CLIBASIA_04310 | | | | | | | | | | | | |
| CLIBASIA_04330 | | | | | | | | 8.206 | | 6.219 | | |
| CLIBASIA_04425 | | | | | | | | | | | | |
| CLIBASIA_04560 | 6.925 | | | | | | | | | | | |
| CLIBASIA_04580 | 0.192 | | 0.369 | | | | 0.732 | 10.62 | | | 1.24 | |
| CLIBASIA_04735 | 0.192 | | | | | | | 0.205 | | | | |
| CLIBASIA_04900 | 7.783 | | | | | | | | | | | |
| CLIBASIA_05150 | | | | | | | | | | | | |
| CLIBASIA_05315 | | | | | | | | 8.472 | | | | |
| CLIBASIA_05320 | | | | | | | | | | | | |
| CLIBASIA_05640 | 10.33 | | | | | | | | | | | |
| 16s Ct | 22.71 | 30.35 | 30.93 | 31.47 | 25.99 | 27.38 | 25.94 | 19.78 | 22.83 | 24.96 | 25.37 | 25.21 |

TABLE 3B

| | Duncan | | | | | | | | | | | |
|---|---|---|---|---|---|---|---|---|---|---|---|---|
| Genes | 6 h | 6 h | 6 h | 24 h | 24 h | 24 h | 3 d | 3 d | 3 d | 7 d | 7 d | 7 d |
| CLIBASIA_00420 | | | 0.092 | | | | | | | | | |
| CLIBASIA_00460 | | 3.657 | 2.779 | | | | | | | 4.482 | | |
| CLIBASIA_00525 | | | | | | | | | | | | |
| CLIBASIA_00530 | | | | | | | | | | | | |
| CLIBASIA_01640 | | | | | | | | | | | | |
| CLIBASIA_02470 | | | | | | | | | 10.9 | 5.18 | | |
| CLIBASIA_03695 | 22.27 | 12.65 | 0 | 4.204 | | | | | 10.31 | | | |
| CLIBASIA_03875 | | | | | | | | | | | | |
| CLIBASIA_04025 | | 0.061 | 0.214 | 0.142 | | | | | | | | |
| CLIBASIA_04310 | | | | | | | | | | | | |
| CLIBASIA_04330 | | | | | | | | | | | | |
| CLIBASIA_04425 | | | | | | | | | | | | |
| CLIBASIA_04560 | | | | | | | | | | | | |
| CLIBASIA_04580 | | | | 0.09 | | | | | | | | |
| CLIBASIA_04735 | | | | | | | | | | | | |
| CLIBASIA_04900 | | | | | | | | | | | | |
| CLIBASIA_05150 | | | | | | | | | | | | |
| CLIBASIA_05315 | 5.878 | 0.61 | 1.176 | 4.017 | 3.862 | 2.744 | 0.697 | 0.883 | 3.005 | 0.76 | 0.809 | 0.782 |
| CLIBASIA_05320 | | | | | | | | | | | | |
| CLIBASIA_05640 | | | | 6.295 | | | | | | | | |
| 16s Ct | 28.13 | 28.5 | 30.2 | 35.63 | 30.56 | 30.72 | 27.47 | 26.8 | 24.99 | 24.5 | 23.92 | 23.48 |

TABLE 30

| | Cleopatra | | | | | | | | | | | |
|---|---|---|---|---|---|---|---|---|---|---|---|---|
| Genes | 6 h | 6 h | 6 h | 24 h | 24 h | 24 h | 3 d | 3 d | 3 d | 7 d | 7 d | 7 d |
| CLIBASIA_00420 | | | | | | | | | | | | |
| CLIBASIA_00460 | | | | 23.26 | 5.06 | | | 2.754 | | | | |
| CLIBASIA_00525 | | | | | | | | | | 2.763 | | 3.377 |
| CLIBASIA_00530 | | | | | | | | | | | | |
| CLIBASIA_01640 | | | | | | | | | | | | |
| CLIBASIA_02470 | | | | | | | | | | | | |
| CLIBASIA_03695 | | | 48.77 | 7.406 | | | 2.355 | | | | | |
| CLIBASIA_03875 | | | | | | | | | | | | |
| CLIBASIA_04025 | | | | | | | | | | | | |
| CLIBASIA_04310 | | | | | | | | | | | | |
| CLIBASIA_04330 | | | | | | | | | | | | |
| CLIBASIA_04425 | | | | | | | | | | | | |
| CLIBASIA_04560 | | | | | | | | | | | | |
| CLIBASIA_04580 | | | | | | | | | | | | |
| CLIBASIA_04735 | | | | | | | | | | | | |
| CLIBASIA_04900 | | | | | | | | 3.065 | | | | |
| CLIBASIA_05150 | | | | | | | | | | | | |
| CLIBASIA_05315 | | | | | 1.788 | 0.673 | 0.619 | 0.273 | 2.431 | 0.422 | 1.608 | 0.58 |
| CLIBASIA_05320 | | | | | 9.021 | | | | | | | |
| CLIBASIA_05640 | | 6.998 | | | | | | 0.086 | | | | |
| 16s Ct | 31.48 | 29.5 | 33.54 | 26.5 | 29.13 | 32.77 | 28.33 | 31.13 | 29.55 | 26.42 | 26.14 | 27.63 |

### Example 7

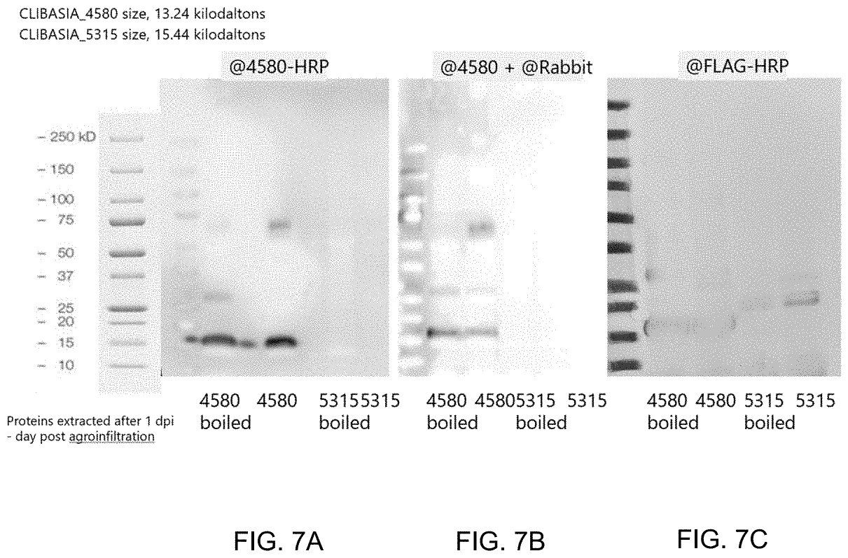

In planta transient expression and detection of Candidatus Liberibacter asiaticus effectors proteins FIGS. 7A-7C show successful in planta transient expression, using agroinfiltration, of CLIBASIA_4580 protein in Nicotiana benthamiana. Protein expression was assessed by observing the presence or absence of CLIBASIA_4580 protein based on size (about 13 Kilodaltons) and detected by western blotting using a rabbit polyclonal CLIBASIA_4580-HRP antibody. The antibodies were conjugated to enzymes horseradish peroxidase (HRP) eliminating the need for the secondary antibody incubation steps. At 1 dpi (day post agroinfiltration) total proteins were extracted from N. benthamiana infiltrated leaves and subjected to western blotting. Transient expression of CLIBASIA_5315 was used as negative control.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1) A kit for detection of citrus huanglongbing (HLB) in a subject in need thereof, comprising two or more antibodies, wherein the two or more antibodies are configured to recognize different amino acid sequences encoding effector proteins from Candidatus Liberibacter asiaticus selected from the group consisting of: SEQ ID NO: 5, 6, 32, 33, 38, 39, 59, 60, 77, 78, 80, 81, 2, 3, 11, 12, 35, 36, 50, 51, 53, 54, 83, 84, or substantially identical variants thereof.

2) The kit of claim 1, wherein one of the antibodies recognizes SEQ ID NO: 32, 33, or substantially identical variants thereof.

3) The kit of claim 1, wherein the kit is configured as a microtiter plate-based assay.

4) The kit of claim 1, wherein the kit is configured as an enzyme-linked immunosorbent assay (ELISA).

5) The kit of claim 4, wherein the kit is configured as a spot-ELISA.

6) The kit of claim 1, wherein the two or more antibodies are detectably labeled.

7) The kit of claim 1, wherein the subject in need thereof is an infected citrus plant or psyllid insect or citrus plant or psyllid insect at risk for infection.

8) The kit of claim 1, wherein one of the two or more antibodies recognizes SEQ ID NO: 32, 33, or substantially identical variants thereof, and the second of the two or more antibodies recognizes SEQ ID NO: 59, 60, or substantially identical variants thereof

9) A method for detection of citrus huanglongbing (HLB) in a subject in need thereof, comprising:
providing a sample from the subject;
administering the sample to the kit of claim 1.

10) The method of claim 9, wherein the subject in need thereof is an infected citrus plant or psyllid insect, or citrus plant or psyllid insect at risk for infection.

11) The method of claim 9, wherein the sample is sap or a leaf.

12) The method of claim 9, wherein one of the two or more antibodies of the kit recognizes SEQ ID NO: 32, 33, or substantially identical variants thereof.

13) The method of claim 9, wherein one of the two or more antibodies of the kit recognizes SEQ ID NO: 32, 33, or substantially identical variants thereof, and the second of the two or more antibodies of the kit recognizes SEQ ID NO: 59, 60, or substantially identical variants thereof.

14) The method of claim 11, wherein the sap or leaf is from a plant selected from the group consisting of individuals or hybrids of: C. aurantium, C. tangerine, C. ichangensis, C. limetta, C. unshiu, C. maxima, C. grandis, C. Paradisi, C. maxima, C. limn C. japonica, C. glauca, C. bergamia C. sinensis, C. reticulata, Poncirus trifoliate.

15) A method for detection of citrus huanglongbing (HLB) in a subject in need thereof, comprising:
providing a sample;
extracting proteins from the sample;
administering two or more antibodies to the extracted proteins, wherein the two or more antibodies are configured to recognize different amino acid sequences encoding effector proteins from Candidatus Liberibacter asiaticus selected from the group consisting of: SEQ ID NO: 5, 6, 32, 33, 38, 39, 59, 60, 77, 78, 80, 81, 2, 3, 11, 12, 35, 36, 50, 51, 53, 54, 83, 84, or substantially identical variants thereof;
forming a plurality of immunocomplexes by incubating the mixture of extracted proteins and two or more antibodies;
detecting the immunocomplexes;
determining from the detected immunocomplexes a disease status; and
outputting the disease status.

16) The method of claim 15, wherein one of the antibodies recognizes SEQ ID NO: 32, 33, or substantially identical variants thereof.

17) The method of claim 15, wherein the two or more antibodies are detectably labeled.

18) The method of claim 15, wherein the subject in need thereof is an infected citrus plant or psyllid insect or citrus plant or psyllid insect at risk for infection.

19) The method of claim 15, wherein one of the two or more antibodies recognizes SEQ ID NO: 32, 33, or substantially identical variants thereof, and the second of the two or more antibodies recognizes SEQ ID NO: 59, 60, or substantially identical variants thereof.

20) The method of claim 15, wherein the sample is sap or a leaf.
